SEQUENCE LISTING (5 sequences)

SEQ ID NO: 1 - DNA, 19 bp, Artificial Sequence
  Description of Artificial Sequence Synthetic oligonucleotide
gagatctggacagaatcgc

SEQ ID NO: 2 - DNA, 21 bp, Artificial Sequence
  Description of Artificial Sequence Synthetic oligonucleotide
gcttgctccctacgatgagaa

SEQ ID NO: 3 - DNA, 19 bp, Artificial Sequence
  Description of Artificial Sequence Synthetic oligonucleotide
gacacgcgacttgtaccac

SEQ ID NO: 4 - DNA, 20 bp, Artificial Sequence
  Description of Artificial Sequence Synthetic oligonucleotide
agacgtgaaccagcaggagt

SEQ ID NO: 5 - DNA, 20 bp, Artificial Sequence
  Description of Artificial Sequence Synthetic oligonucleotide
ttctctgacgtcccccatag

(19) United States
(12) Patent Application Publication
(10) Pub. No.: US 2024/0325382 A1
(43) Pub. Date: Oct. 3, 2024

(54) COMPOSITIONS AND METHODS FOR TREATING ANEMIA ASSOCIATED WITH A RIBOSOMAL DISORDER

(71) Applicant: Disc Medicine, Inc., Watertown, MA (US)

(72) Inventors: Brian Richard MacDonald, Watertown, MA (US); William Jacob Savage, Watertown, MA (US); Maria Gabriela Beconi, Watertown, MA (US); Min Wu, Watertown, MA (US); Pavan Reddy, Watertown, MA (US)

(21) Appl. No.: 18/280,774

(22) PCT Filed: Mar. 11, 2022
(86) PCT No.: PCT/US2022/020039

§ 371 Date: Sep. 7, 2023

Related U.S. Application Data
(60) Provisional application No. 63/185,466, filed on May 7, 2021.
(60) Provisional application No. 63/160,413, filed on Mar. 12, 2021.

(51) Int. Cl. A61K 31/496; A61K 45/06

(52) U.S. Cl. CPC A61K 31/496; A61K 45/06

(57) ABSTRACT

The present embodiments are directed to methods of using glycine transporter inhibitors, such as GlyT1 inhibitors, or pharmaceutically acceptable salts, solvates or prodrugs thereof, or pharmaceutical compositions thereof, for preventing or treating anemia associated with a ribosomal disorder, and related syndromes thereof.

### CROSS-REFERENCE TO RELATED APPLICATIONS

This application claims the benefit of priority to U.S. provisional application Ser. No. 63/160,413, filed on Mar. 12, 2022; and 63/185,466, filed on May 7, 2021. The disclosures of the foregoing applications are hereby incorporated by reference in their entirety.

### FIELD

Embodiments disclosed herein are directed to methods and uses to prevent or treat anemia associated with a ribosomal disorder with glycine transporter inhibitors, such as, but not limited to, GlyT1 inhibitors, or pharmaceutically acceptable salts, solvates, prodrugs thereof, or pharmaceutical compositions thereof.

### BACKGROUND

Mutations in ribosomal protein (RP) genes or other transcription factors (e.g., GATA1) can result in the loss of erythrocyte progenitor cells and cause anemia associated with a ribosomal disorder. One example of an anemia associated with a ribosomal disorder is Diamond-Blackfan anemia (DBA), a rare blood disorder that is almost exclusively linked to RP gene haploinsufficiency. DBA affects approximately seven per million live births and is usually diagnosed during the first year of life. Classic diagnostic criteria includes: (1) macrocytic, normochromic, anemia; (2) reticulocytopenia; (3) bone marrow erythroid hypoplasia; and (4) early onset of anemia (90% present before age one year).

In DBA patients, erythrocyte precursors do not mature sufficiently leading to congenital erythroid aplasia, developmental defects and increased risk of myelodysplastic syndrome or acute myeloid leukemia. Affected individuals may have physical abnormalities, such as craniofacial malformations, thumb or upper limb abnormalities, cleft palate, as well as defects of the genitalia, urinary tract, eyes and heart. In some cases, low birth weight and short stature are observed. DBA patients are also at a modest risk of developing leukemia and other malignancies.

The current treatment options for DBA includes corticosteroids, blood transfusion, and bone marrow transplantation. Approximately 80% of DBA patients respond to an initial course of corticosteroids. However, the efficacy of corticosteroids can wane over time in many patients. These patients and the 20% who do not respond initially to such therapy must be maintained on a chronic blood transfusion with iron chelation. Chronic transfusions are known to cause iron overload in various organs including the liver, heart, and endocrine system. Other therapies such as interleukin-3, high dose corticosteroids, cyclosporine, anti-thymocyte globulin, immunoglobulin, and metoclopramide, are either of unproved benefit and/or seem to benefit relatively few people. Pharmacological doses of Erythropoietin (EPO) are also ineffective. Bone marrow transplantation is the sole cure for the hematologic manifestation of DBA-related anemia, but is usually only considered in corticosteroid-resistant persons because of substantial morbidity and mortality. Typically, only transplants from human leukocyte antigen (HLA)-identical sibling were considered. For many patients, the lack of a suitable donor excludes bone marrow transplantation as a therapeutic option.

As such, there is a high, unmet need for effective therapies for treating anemias associated with ribosomal disorders. Accordingly, it is an object of the present disclosure to provide methods for treating, preventing, or reducing the progression rate and/or severity of anemia associated with a ribosomal disorder. The methods and use of glycine transporter inhibitors, such as, but not limited to, GlyT1 inhibitors, described herein fulfill these needs as well as others.

### SUMMARY OF THE APPLICATION

In some embodiments, the disclosure provides for a method of treating anemia associated with a ribosomal disorder in a subject, the method comprising administering to the subject a pharmaceutical composition comprising one or more glycine transporter 1 (GlyT1) inhibitor, or a pharmaceutically acceptable salt thereof, or a prodrug of the one or more GlyT1 inhibitor or its salt. In some embodiments, the disclosure provides for a method of preventing, treating, or reducing the progression rate and/or severity of anemia associated with a ribosomal disorder in a subject, the method comprising administering to the subject a pharmaceutical composition comprising one or more glycine transporter 1 (GlyT1) inhibitor, or a pharmaceutically acceptable salt thereof, or a prodrug of the one or more GlyT1 inhibitor or its salt.

In some embodiments, the disclosure provides for a method of preventing, treating, or reducing the progression rate and/or severity of one or more complications of anemia associated with a ribosomal disorder in a subject, the method comprising administering to the subject a pharmaceutical composition comprising one or more GlyT1 inhibitor, or a pharmaceutically acceptable salt thereof, or a prodrug of the one or more GlyT1 inhibitor or its pharmaceutically acceptable salt. In some embodiments, the one or more complications of anemia associated with a ribosomal disorder is selected from the group consisting of: thrombocytosis, megakaryotypic hyperplasia, infections, bleeding (e.g., from the nose or gums), bruising, splenomegaly, the need for more frequent blood transfusions, the need for increased glucocorticoid use, the need for allogenic hematopoietic stem cell transplantation, the need for autologous gene therapy, marrow failure, MDS, leukemia, and acute myelogenous leukemia.

In some embodiments, the anemia associated with a ribosomal disorder is Diamond-Blackfan anemia. In some embodiments, the subject is haploinsufficient for a ribosomal protein selected from the group consisting of 40S ribosomal protein S14 (RPS14), 40S ribosomal protein S19 (RPS19), 40S ribosomal protein S24 (RPS24), 40S ribosomal protein S17 (RPS17), 60S ribosomal protein L35a (RPL35a), 60S ribosomal protein L5 (RPL5), 60S ribosomal protein L11 (RPL11), and 40S ribosomal protein S7 (RPS7). In some embodiments, the subject is haploinsufficient for a ribosomal protein selected from the group consisting of 40S ribosomal protein S10 (RPS10), 40S ribosomal protein S26 (RPS26), 60S ribosomal protein L15 (RPL15), 60S ribosomal protein L17 (RPL17), 60S ribosomal protein L19 (RPL19), 60S ribosomal protein L26 (RPL26), 60S ribosomal protein L27 (RPL27), 60S ribosomal protein L31 (RPL31), 40S ribosomal protein S15a (RPS15a), 40S ribosomal protein S20 (RPS20), 40S ribosomal protein S27 (RPS27), 40S ribosomal protein S28 (RPS28), and 40S ribosomal protein S29 (RPS29). In some embodiments, the subject has one or more mutations in a ribosomal protein gene. In some embodiments, the subject has one or more mutations in a ribosomal protein gene selected from the group consisting of RPL5, RPL9, RPL11, RPL15, RPL17, RPL18, RPL19, RPL26, RPL27, RPL31, RPL35a, RPS7, RPS10, RPS14, RPS15a, RPS15, RPS17, RPS19, RPS20, RPS24, RPS26, RPS27a, RPS27, RPS28, and RPS29. In some embodiments, the subject has one or more mutations in a non-ribosomal protein gene selected from the group consisting of TSR2, GATA1, and EPO.

In some embodiments, the anemia associated with a ribosomal disorder is myelodysplastic syndrome associated (MDS) with isolated del(5q). In some embodiments, the subject has low risk, intermediate-1, intermediate-2, or high risk MDS as classified by the International Prognostic Scoring System (IPSS). In some embodiments, the subject is haploinsufficient for a ribosomal protein selected from the group consisting of 40S ribosomal protein S14 (RPS14) and 40S ribosomal protein S19 (RPS19). In some embodiments, the subject has one or more mutations in a ribosomal protein gene. In some embodiments, the one or more mutations in a ribosomal protein gene are selected from the group consisting of RPS14 or RPS19.

In some embodiments, the anemia associated with a ribosomal disorder is Shwachman-Diamond syndrome. In some embodiments, the subject has one or more mutations in the SBDS gene. In some embodiments, the method decreases the need for hematopoietic stem cell transplant in the subject. In some embodiments, the method decreases neutropenia in the subject. In some embodiments, the method decreases thrombocytopenia in the subject. In some embodiments, the method decreases the subject's risk of developing myelodysplastic syndrome. In some embodiments, the method decreases the subject's risk of developing leukemia. In some embodiments, the method decreases the subject's risk of developing an infection. In some embodiments, the method decreases the subject's risk of developing pneumonia.

In some embodiments, the anemia associated with a ribosomal disorder is dyskeratosis congenita. In some embodiments, the dyskeratosis congenita is x-linked dyskeratosis congenita. In some embodiments, the subject has one or more mutations in the DKC1 gene. In some embodiments, the subject has one or more mutations in a gene selected from the group consisting of TINF2, TERC, TERT, C16orf57, NOLA2, NOLA3, WRAP53/TCAB1, PARN, CTC1, and RTEL1. In some embodiments, the method decreases the risk of bone marrow failure in the subject. In some embodiments, the method decreases the risk of pulmonary fibrosis in the subject. In some embodiments, the method decreases the risk of liver fibrosis in the subject. In some embodiments, the anemia associated with a ribosomal disorder is cartilage hair hypoplasia. In some embodiments, the subject has one or more mutations in the RMRP gene.

In some embodiments, the method reduces the need for bone marrow transplantation in the subject. In some embodiments, the subject has elevated heme levels. In some embodiments, the subject has decreased erythroid precursor survival as compared to a healthy subject. In some embodiments, the subject has decreased erythroid precursor differentiation into mature red blood cells as compared to a healthy subject. In some embodiments, the subject has a low red blood cell count. In some embodiments, the subject has impaired hematopoiesis. In some embodiments, the subject has impaired 40S ribosomal subunit maturation. In some embodiments, the subject has impaired 60S ribosomal subunit maturation. In some embodiments, the subject has decreased hemoglobin levels. In some embodiments, the subject has decreased hematocrit levels. In some embodiments, the subject has a low quality of life. In some embodiments, the subject has liver iron overload. In some embodiments, the subject has cardiac iron overload. In some embodiments, the subject has increased spleen size. In some embodiments, the anemia is due to a failure in erythropoiesis. In some embodiments, the subject has elevated erythrocyte adenosine deaminase activity. In some embodiments, the subject has increased red cell adenosine deaminase. In some embodiments, the subject has macrocytic anemia. In some embodiments, the subject has reticulocytopenia. In some embodiments, the subject has a reticulocyte count of less than 1%. In some embodiments, the subject has normal marrow cellularity with a paucity of red cell precursors. In some embodiments, the subject has normal neutrophil and/or platelet counts. In some embodiments, the subject has elevated fetal hemoglobin levels. In some embodiments, the subject has increased fetal hemoglobin content in red cells. In some embodiments, the subject has decreased red cell mass. In some embodiments, the subject has an increased mean corpuscular volume of red cells.

In some embodiments, the subject has heme levels that are at least 10%, 20%, 30%, 40%, 50%, 60%, 70%, 80%, 90%, or 100% more than heme levels in a healthy subject prior to administration of the GlyT1 inhibitor. In some embodiments, the method reduces the heme levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method reduces heme synthesis in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method reduces intracellular heme levels. In some embodiments, the method reduces intracellular heme levels in erythroid precursors. In some embodiments, the subject has a red blood cell count that is at least 10%, 20%, 30%, 40%, 50%, 60%, 70%, 80%, or 90% less than a red blood cell count in a healthy subject prior to administration of the GlyT1 inhibitor. In some embodiments, the method increases the subject's red blood cell count. In some embodiments, the method increases the subject's red blood cell count by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

In some embodiments, the subject has hemoglobin levels that are at least 10%, 20%, 30%, 40%, or 50% less than hemoglobin levels in a healthy subject prior to administration of the GlyT1 inhibitor. In some embodiments, the subject has hemoglobin levels that are less than 13 g/dL. In some embodiments, the subject has hemoglobin levels that are less than 11 g/dL. In some embodiments, the method increases the subject's hemoglobin levels. In some embodiments, the method increases the subject's hemoglobin levels by at least 10% (e.g., 10%, 15%, 20%, 25%, 30%, 35%, 40%, 45%, 50%, 55%, 60%, 65% 7%, 80%, 85%, 90%, 95%, or at least 100%). In some embodiments, the method increases the subject's hemoglobin levels to at least 13 g/dL. In some embodiments, the method increases the subject's hemoglobin levels to at least 11 g/dL.

In some embodiments, the subject has hematocrit levels that are at least 10%, 20%, 30%, 40%, 50%, 60%, 70%, 80%, or 90% less than hematocrit levels in a healthy subject prior to administration of the GlyT1 inhibitor. In some embodiments, the subject has hematocrit levels that are less than 38%. In some embodiments, the subject has hematocrit levels that are less than 35%. In some embodiments, the method increases the subject's hematocrit levels. In some embodiments, the method increases the subject's hematocrit levels by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method increases the subject's hematocrit levels to at least 38%. In some embodiments, the method increases the subject's hematocrit levels to at least 35%.

In some embodiments, the method reduces anemia in the subject. In some embodiments, the method reduces anemia in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method increases the subject's reticulocyte count. In some embodiments, the method increases the subject's reticulocyte count to between 1% to 2%. In some embodiments, the method increases the subject's erythroid precursor survival. In some embodiments, the method increases the subject's erythroid precursor survival by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method increases erythroid precursor differentiation into mature red blood cells in the subject. In some embodiments, the method increases erythroid precursor differentiation into mature red blood cells in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

In some embodiments, the method reduces the risk of heme toxicity in the subject. In some embodiments, the method reduces the risk of heme toxicity by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method reduces the risk of liver iron overload. In some embodiments, the method reduces the levels of iron in the liver. In some embodiments, the method reduces the levels of iron in the liver by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method reduces the risk of cardiac iron overload. In some embodiments, the method reduces the level of iron in the heart. In some embodiments, the method reduces the levels of iron in the heart by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

In some embodiments, the subject has an increased spleen size. In some embodiments, the method reduces the subject's spleen size. In some embodiments, the method reduces the subject's spleen size by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method reduces the subject's need for blood transfusions. In some embodiments, the method reduces the subject's need for blood transfusions by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method eliminates the subject's need for blood transfusions.

In some embodiments, the method increases the subject's quality of life. In some embodiments, the method increases the subject's quality of life by at least 1% (e.g., 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%). In some embodiments, the subject's quality of life is measured using an assessment selected from the group consisting of the Functional Assessment of Cancer Therapy Anemia (FACT-An), Functional Assessment of Cancer Therapy Fatigue (FACT-Fatigue), Functional Assessment of Chronic Illness Therapy (FACIT), the Functional Assessment of Chronic Illness Therapy Fatigue (FACIT-Fatigue), Functional Assessment of Chronic Illness Therapy Anemia (FACIT-Anemia), the SF-36 generic PRO tool, the SF-6D generic PRO tool, and the linear analog scale assessment (LASA).

In some embodiments, the method reduces the need for corticosteroid treatments in the subject. In some embodiments, the method reduces the dose of corticosteroid treatment needed in the subject. In some embodiments, the corticosteroid is a glucocorticoid steroid. In some embodiments, the method increases survival by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method further comprises administering to the subject an additional active agent and/or supportive therapy. In some embodiments, the additional active agent and/or supportive therapy is selected from the group consisting of: trifluoperazine, lenalidomide, HDAC inhibitors, glucocorticoids, sotatercept, luspatercept, iron chelators, blood transfusion, platelet transfusion, allogeneic hematopoietic stem cell transplant, autologous gene therapy, and antibiotics.

In certain embodiments, the GlyT1 inhibitor is a compound of Formula I,

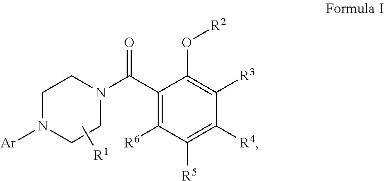

wherein Ar is unsubstituted or substituted aryl or 6-membered heteroaryl containing one, two or three nitrogen atoms, wherein the substituted aryl and the substituted heteroaryl groups are substituted by one or more substituents selected from the group consisting of hydroxy, halogen, NO₂, CN, (C₁-C₆)-alkyl, (C₁-C₆)-alkyl substituted by halogen, (C₁-C₆)-alkyl substituted by hydroxy, (CH₂)n-(C₁-C₆)-alkoxy, (C₁-C₆)-alkoxy substituted by halogen, NR⁷R⁸, C(O)R⁹, SO2R¹⁰, and —C(CH₃)═NOR⁷, or are substituted by a 5-membered aromatic heterocycle containing 1-4 heteroatoms selected from N and O, which is optionally substituted by (C₁-C₆)-alkyl; R¹ is hydrogen or (C₁-C₆)-alkyl; R² is hydrogen, (C₁-C₆)-alkyl, (C₂-C₆)-alkenyl, (C₁-C₆)-alkyl substituted by halogen, (C₁-C₆)-alkyl substituted by hydroxy, (CH2)n (C₃-C₇)-cycloalkyl optionally substituted by (C₁-C₆)-alkoxy or by halogen, CH(CH₃)—(C₃-C₇)-cycloalkyl, (CH₂)ₙ₊₁—C(O)—R⁹, (CH₂)ₙ₊₁—CN, bicyclo[2.2.1]heptyl, (CH₂)ₙ₊₁—O—(C₁-C₆)-allyl, (CH₂)ₙ-heterocycloalkyl, (CH₂)ₙ-aryl or (CH₂)ₙ-5 or 6-membered heteroaryl containing one, two or three heteroatoms selected from the group consisting of oxygen, sulphur or nitrogen wherein aryl, heterocycloalkyl and heteroaryl are unsubstituted or substituted by one or more substituents selected from the group consisting of hydroxy, halogen, (C₁-C₆)-alkyl and (C₁-C₆)-alkoxy; R³, R⁴ and R⁶ are each independently hydrogen, hydroxy, halogen, (C₁-C₆)-alkyl, (C₁-C₆)-alkoxy or O—(C₃-C₆)-cycloalkyl; R⁵ is NO₂, CN, C(O)R⁹ or SO₂R¹⁰; R⁷ and R⁸ are each independently hydrogen or (C₁-C₆)-alkyl; R⁹ is hydrogen, (C₁-C₆)-alkyl, (C₁-C₆)-alkoxy or NR⁷R⁸; R¹⁰ is (C₁-C₆)-alkyl optionally substituted by halogen, (CH₂)ₙ—(C₃-C₆)-cycloalkyl, (CH₂)ₙ—(C₃-C₆)-alkoxy, (CH₂)ₙ-heterocycloalkyl or NR⁷R⁸; n is 0, 1, or 2; or a pharmaceutically acceptable salt thereof, or a prodrug of the compound or its pharmaceutically acceptable salt.

In certain embodiments, GlyT1 inhibitor is a compound having a formula of

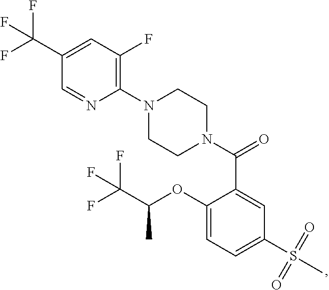 [SMILES: C[C@H](Oc1ccc(S(C)(=O)=O)cc1C(=O)N1CCN(c2ncc(C(F)(F)F)cc2F)CC1)C(F)(F)F]

bitopertin, or a pharmaceutically acceptable salt thereof, or a prodrug of the compound or its pharmaceutically acceptable salt.

In certain embodiments, the GlyT1 inhibitor is a compound of Formula II,

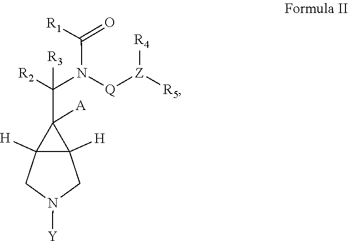

wherein R₁ represents a heteroaryl selected from the group consisting of: imidazolyl, thiazolyl, pyridyl, oxazolyl, pyrazolyl, triazolyl, oxadiazolyl, quinolinyl, isoxazolyl, pyrroloimidazoyl, and thiadiazole, wherein said heteroaryl is optionally substituted by one or more substituents selected from —OH, —NR₇R₈, halogen, (C₁-C₈)alkyl, (C₃-C₁₀)cycloalkyl, (C₁-C₈)alkoxy, (C₁-C₁₂)alkoxyalkyl, (C₁-C₈)hydroxyalkyl, (C₆-C₁₄)aryl and benzyl; R₂, R₃ and A independently represent H or (C₁-C₈)alkoxy, wherein said alkyl is optionally substituted by one or more —OH, (C₁-C₈)alkoxy, —NR₇R₈ or halogen; Q represents —(CH₂)ₙ—, where n=1, 2, 3 or 4 or —(CH₂)ₘ—O—, where m=2, 3 or 4; Z represents (C₆-C₁₄)aryl, (C₁-C₈)alkyl or (C₃-C₈)cycloalkyl; R₄ and R₅ each independently represent H, halogen, (C₁-C₈)alkyl, (C₆-C₁₄)aryl, (C₆-C₁₄)aryloxy, (C₁-C₈)alkoxy, (3-10 membered)heterocycloalkyl or (C₃-C₈)cycloalkoxy; wherein R₄ and R₅ are optionally substituted by one or more —OH, (C₁-C₈)alkoxy, —NR₇R₈ or halogen; Y represents —R₆, —(CH₂)o-R₆, —C(R₆)₃ or —CH(R₆)₂, wherein 0=1, 2 or 3; R₆ represents H, (C₆-C₁₄)aryl, (C₁₋₁₀)alkyl, (C₃-C₁₀)cycloalkyl, (C₅-C₁₈)bicycloalkyl, (C₅-C₁₈)tricycloalkyl, (3-10 membered)heterocycloalkyl, (5-10 membered)heteroaryl, —C(═O)NR₇R₈, or —C(═O)OR₇, wherein said R₆ groups can optionally be substituted with one or more X groups; wherein X=—OH, (C₁-C₈)alkoxy, —NR₁₁R₁₂, —SO₂R₁₀, —C(═O)R₁₀, halogen, cyano, (C₁-C₈)alkyl, (C₁-C₁₀)alkoxyalkyl, (5-10 membered)heteroaryl, (C₆-C₁₄)aryl, (C₆-C₁₄)aryloxy, benzyl, or (C1-C₈)hydroxyalkyl; wherein R₇ and R₈ independently represent H, (C₁-C₈)alkyl, (C₃-C8)cycloalkyl, (5-10 membered)heterocycloalkyl, (C₁-C₈)hydroxyalkyl, (5-10 membered)heteroaryl or (C₁-C₁₀)alkoxyalkyl; wherein R₇ and R₈ may optionally be substituted by one or more X groups; or R₇ and R₈ together with the nitrogen in which they may be attached may form a (3-10 membered)heterocycloalkyl group optionally substituted by one or more X groups; wherein R₁₀ represents (C₁-C₈)alkyl, (C₃-C₈)cycloalkyl, (3-10 membered)heterocycloalkyl, (C₁-C₈)hydroxyalkyl, (5-10 membered)heteroaryl or (C₁-C₁₀)alkoxyalkyl; wherein R₁₁ and R₁₂ independently represent H, (C₁-C₈)alkyl, (C₃-C₈)cycloalkyl, (5-10 membered)heterocycloalkyl, (C₁-C₈)hydroxyalkyl, (5-10 membered)heteroaryl or (C₁-C₁₀)alkoxyalkyl; or a pharmaceutically acceptable salt thereof, or a prodrug of the compound or its pharmaceutically acceptable salt. In certain such embodiments, the GlyT1 inhibitor is a compound having a formula of

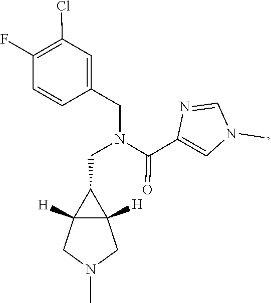 [SMILES: CN1C[C@@H]2[C@H](C1)[C@@H]2CN(Cc1ccc(F)c(Cl)c1)C(=O)c1cn(C)cn1]

or a pharmaceutically acceptable salt thereof, or a prodrug of the compound or its pharmaceutically acceptable salt. In other such embodiments, the GlyT1 inhibitor is a compound having a formula of

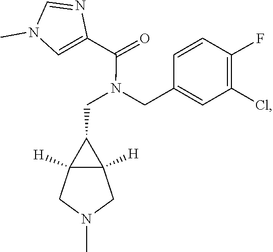 [SMILES: CN1C[C@@H]2[C@H](C1)[C@H]2CN(Cc1ccc(F)c(Cl)c1)C(=O)c1cn(C)cn1]

PF-3463275, or a pharmaceutically acceptable salt thereof, or a prodrug of the compound or its pharmaceutically acceptable salt.

In certain embodiments, the GlyT1 inhibitor is a compound of Formula III,

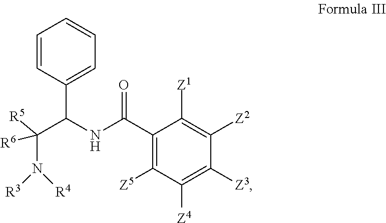

wherein Z¹ is selected from the group consisting of C₁₋₄alkyl, C₃₋₆Cycloalkyl, C₁₋₄alkoxy, C₁₋₄alkylthio, haloC₁₋₄alkyl, phenyl, haloC₁₋₄alkoxy, halophenyl, C₁₋₄alkylsulfoxy, C₁₋₄alkylsulfonyl, bromo and chloro; Z² is selected from the group consisting of hydrogen, halogen, cyano, C₁₋₄alkyl, phenyl, haloC₁₋₄alkyl, haloC₁₋₄ alkoxy, halophenyl, C₁₋₄alkoxyC₁₋₄alkyl and C₃₋₆cycloalkyl; Z³ is selected from the group consisting of hydrogen, halogen, C₁₋₄alkyl, C₁₋₄alkoxy, C₁₋₄alkylthio, haloC₁₋₄alkyl, haloC₁₋₄alkoxy, and C₃₋₆cycloalkyl; Z⁴ is selected from the group consisting of hydrogen, halogen, C1-3alkyl, haloC₁₋₄alkyl, C₁₋₄alkoxy, C₁₋₄alkylthio, phenyl, haloC₁₋₄alkoxy, halophenyl, C₁₋₄alkoxyC₁₋₄alkyl and C₃₋₆cycloalkyl; Z⁵ is selected from the group consisting of hydrogen, fluoro, chloro, bromo, iodo, hydroxy, C₁₋₄alkyl, C₁₋₄alkoxy, C₁₋₄alkylthio, phenyl, haloC₁₋₄alkyl, haloC₁₋₄alkoxy, halophenyl, C₁₋₄alkoxyC₁₋₄alkyl and C₃₋₆cycloalkyl; whereby if more than one of Z¹ to Z⁵ is methoxy, then only Z¹ and Z⁵ are methoxy R³ and R⁴ are independently selected from hydrogen and C₁₋₄alkyl, optionally substituted with one or more groups Y; or R³ and R4 together with the nitrogen atom to which they are attached form a saturated or partially unsaturated A-, 5- 6- or 7-membered carbocyclic ring optionally substituted with a group Y′; Y is selected from the group consisting of C₁₋₄alkoxy, hydroxy, haloC₁₋₄alkoxy and C₃₋₅cycloalkyl; Y′ is selected from the group consisting of C₁₋₄alkyl, C₁₋₄alkoxy, halogen, hydroxy, haloC₁₋₄alkoxy, C₃₋₅cycloalkyl and C₅₋₁₀aryl or Y′ forms a —CH2- or —CH2-CH2- bridge between two atoms on the A-, 5-, 6- or 7-membered carbocyclic ring; R⁵ and R⁶ are independently C₁₋₄alkyl, optionally substituted with one or more groups X; or R⁵ and R⁶ together with the carbon atom to which they are attached form a saturated 5- or 6-membered ring carbocyclic optionally substituted with one or more groups X′, in the case of R⁵ and R6 together with the carbon atom to which they are attached forming a 5-membered saturated carbocyclic ring, that ring may optionally further comprising an additional heteroatom group selected from O, N and S(O)m, where m=0, 1 or 2; X is selected from the group consisting of halogen, hydroxy, C₁₋₄alkoxy, haloC₁₋₄alkyl, haloC₁₋₄alkoxy and C₅₋₁₀aryl; and X′ is selected from the group consisting of halogen, hydroxy, C₁₋₄alkyl, C₁₋₄alkoxy, haloC₁₋₄alkyl, haloC₁₋₄alkoxy and C₅₋₁₀aryl; whereby R³, R⁴, R⁵ and R⁶ are not all simultaneously unsubstituted methyl; with the provisos that when simultaneously Z¹ is propyloxy, Z³ is chloro, Z²=Z⁴=Z⁵═H, and R⁵ and R⁶ are both methyl, then R³ and R⁴ together with the nitrogen atom to which they are attached do not form a 2-methylpyrrolidine group; when simultaneously Z¹ is methyl, Z³ is methoxy, Z²=Z4=Z5=H, and R⁵ and R⁶ are both methyl, then R³ and R⁴ together with the nitrogen atom to which they are attached do not form a pyrrolidine group, or a pharmaceutically acceptable salt thereof, or a prodrug of the compound or its pharmaceutically acceptable salt. In certain such embodiments, the GlyT1 inhibitor is a compound having a formula of

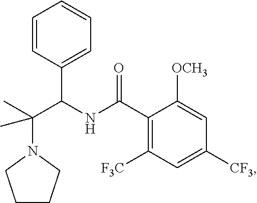 [SMILES: COc1cc(C(F)(F)F)cc(C(F)(F)F)c1C(=O)NC(c1ccccc1)C(C)(C)N1CCCC1]

or a pharmaceutically acceptable salt thereof, or a prodrug of the compound or its pharmaceutically acceptable salt.

In certain embodiments, the GlyT1 inhibitor is a compound of Formula IV,

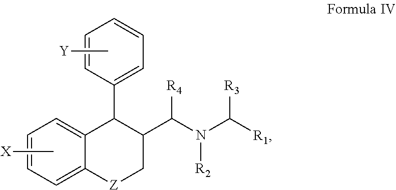

wherein Z is (CH₂)ₙ, O, S, SO, SO₂ or N—R₅; n is 0, 1 or 2; X represents 1-3 substituents independently selected from hydrogen, halogen, (C₁₋₆)alkyloxy, (C₃₋₆)cycloalkyloxy, (C₆₋₁₂)aryloxy, (C₆₋₁₂)aryl, thienyl, SR₆, SOR₆, SO₂R₆, NR₆R₆, NHR₆, NH₂, NHCOR₆, NSO₂R₆, CN, COOR₆ and (C₁₋₄)alkyl, optionally substituted with halogen, (C₆₋₁₂)aryl, (C₁₋₆)alkyloxy or (C₆₋₁₂)aryloxy; or 2 substituents at adjacent positions together represent a fused (C₅₋₆)aryl group, a fused (C₅₋₆)cycloalkyl ring or 0-(CH₂)ₘ—O; m is 1 or 2; Y represents 1-3 substituents independently selected from hydrogen, halogen, (C₁₋₄)alkyloxy, SR₆, NR₆R₆ and (C₁₋₄)alkyl, optionally substituted with halogen; R₁ is COOR₇ or CONR₈R₉; R₂ and R₆ are (C₁₋₄)alkyl; R₃, R₄ are R₅ are independently hydrogen or (C₁₋₄)alkyl; R₇, R₈ and R₉ are independently hydrogen, (C₁₋₄)alkyl, (C₆₋₁₂)aryl or arylalkyl, or a pharmaceutically acceptable salt thereof, or a prodrug of the compound or its pharmaceutically acceptable salt. In certain such embodiments, the GlyT1 inhibitor is a compound having a formula of

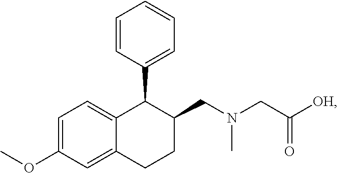 [SMILES: COc1ccc2c(c1)CC[C@H](CN(C)CC(=O)O)[C@@H]2c1ccccc1]

or a pharmaceutically acceptable salt thereof, or a prodrug of the compound or its pharmaceutically acceptable salt.

In certain embodiments, the GlyT1 inhibitor is a compound of Formula V,

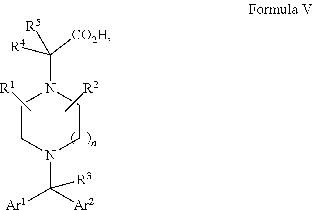

wherein n is an integer from 1 to 3; R¹ and R² are independently selected from hydrogen, alkyl, haloalkyl, alkoxy, haloalkoxy, aryl, heteroaryl, cycloalkyl, or heterocyclyl wherein the aforementioned rings are optionally substituted with R^{a}, R^{b}, or R^{c} independently selected from alkyl, halo, haloalkyl, alkoxy, haloalkoxy, hydroxy, cyano, monosubstituted amino, or disubstituted amino; or R¹ and R², when attached to the same carbon atom, can combine to form cycloalkyl or monocyclic saturated heterocyclyl to give a spiro ring wherein the cycloalkyl or monocyclic saturated heterocyclyl can be optionally substituted with R^{d}, R^{e}, or R^{f} independently selected from alkyl, alkoxy, fluoro, fluoroalkyl, fluoroalkoxy, hydroxy, monosubstituted amino, or disubstituted amino; or R¹ and R², when attached to carbon atoms 2 and 5 or 3 and 6 positions of the piperazine ring, can combine to form —C₁-C₃— alkylene chain wherein one of the carbon atoms in the alkylene chain is optionally replaced by a —NR—, —O—, —S(O)n- (where R is hydrogen or alkyl and n is 0-2) and further wherein one or two hydrogen atoms in the alkylene chain can be optionally substituted with one or two alkyl; R³, R⁴ and R⁵ are independently hydrogen, alkyl, fluoro, or fluoroalkyl; and Ar¹ and Ar² are independently aryl, heteroaryl, cycloalkyl, or heterocyclyl where each of the aforementioned ring is optionally substituted with R^{g}, R^{h} or Rⁱ where R^{g} is alkyl, —C≡C—R⁶ (where R⁶ is aryl or heteroaryl), halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{h} and Rⁱ are independently selected from alkyl, halo, haloalkyl, haloalkoxy, allylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, or heterocyclyl where the aromatic or alicyclic ring-in R^{g}, R^{h} and Rⁱ is optionally substituted with R^{j}, R^{k}, or R^{l} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino; or a pharmaceutically acceptable salt thereof provided that: the compound of Formula V is not 2-(4-benzhydrylpiperazin-1-yl)acetic acid, 2-(4-((4-chlorophenyl)(phenyl)methyl)piperazin-1-yl)acetic acid, 2-((2R,5S)-4-((R)-(4-(1H-tetrazol-5-yl)phenyl)(3-hydroxyphenyl)methyl)-2,5-dimethylpiperazin-1-yl)acetic acid, or 2-((2R,5S)-4-((R)-(4-cyanophenyl)(3-hydroxyphenyl)methyl)-2,5-dimethylpiperazin-1-yl)acetic acid, or a pharmaceutically acceptable salt thereof, or a prodrug of the compound or its pharmaceutically acceptable salt. In certain such embodiments, the GlyT1 inhibitor is a compound having a formula of

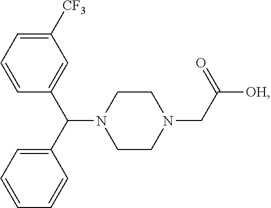 [SMILES: O=C(O)CN1CCN(C(c2ccccc2)c2cccc(C(F)(F)F)c2)CC1]

or a pharmaceutically acceptable salt thereof, or a prodrug of the compound or its pharmaceutically acceptable salt.

In certain embodiments, the GlyT1 inhibitor is a compound of Formula VI,

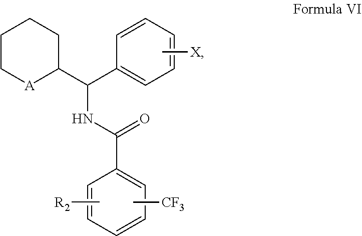

wherein A represents a group of general formula N—R₁, a group of general formula N+(O—)R₁ or a group of general formula N+(R′)R₁, and in which R₁ represents either a hydrogen atom, or a linear or branched (C₁-C₇)alkyl group optionally substituted with one or more fluorine atoms, or a (C₄-C₇)cycloalkyl group, or a (C₃-C₇)cycloalkyl(C₁-C₃)alkyl group, or a phenyl(C₁-C₃)alkyl group optionally substituted with one or two hydroxyl or methoxy groups, or a (C₂-C₄)alkenyl group, or a (C₂-C₄)alkynyl group; R′ represents a linear or branched (C₁-C₇)alkyl group; X represents a hydrogen atom or one or more substituents chosen from halogen atoms and trifluoromethyl, linear or branched (C₁-C₄)alkyl and (C₁-C₄)alkoxy groups; R₂ represents either a hydrogen atom, or one or more substituents chosen from halogen atoms and trifluoromethyl, (C₁-C₄)alkyl or (C₁-C₄)alkoxy groups, or amino groups of general formula NR₃R₄ in which R₃ and R₄ each represent, independently of each other, a hydrogen atom or a (C₁-C₄)alkyl group, or form with the nitrogen atom carrying them a pyrrolidine, piperidine or morpholine ring, or a phenyl group optionally substituted with an atom or a group as defined for the symbol X above, or a pharmaceutically acceptable salt thereof, or a prodrug of the compound or its pharmaceutically acceptable salt. In certain such embodiments, the GlyT1 inhibitor is a compound having a formula of

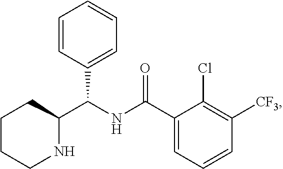 [SMILES: O=C(N[C@@H](c1ccccc1)[C@@H]1CCCCN1)c1cccc(C(F)(F)F)c1Cl]

or a pharmaceutically acceptable salt thereof, or a prodrug of the compound or its pharmaceutically acceptable salt.

In certain embodiments, the GlyT1 inhibitor is a compound of Formula VII,

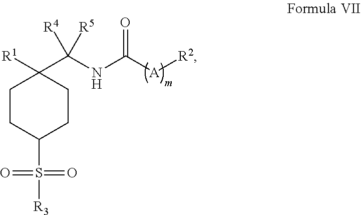

wherein R¹ is (CH₂)ₙ—R^{1a}, wherein n is independently 0-6, and R^{1a} is selected from the group consisting of: (1) C₁₋₆alkyl, which is unsubstituted or substituted with 1-6 halogen, hydroxy, (2) phenyl substituted with R^{2a}, R^{2b} and R^{2c}, (3) C₃₋₆cycloalkyl, which is unsubstituted or substituted with C₁₋₆alkyl, 1-6 halogen, hydroxy or —NR¹⁰R¹¹, (4) —O—C₁₋₆alkyl, which is unsubstituted or substituted with 1-6 halogen, hydroxy or —NR¹⁰R¹¹, (5) —CO2R⁹, wherein R9 is independently selected from: (a) hydrogen, (b) —C₁₋₆alkyl, which is unsubstituted or substituted with 1-6 fluoro, (c) benzyl, and (d) phenyl, (6) —NR¹⁰R¹¹, wherein R¹⁰ and R¹¹ are independently selected from: (a) hydrogen, (b) C₁₋₆alkyl, which is unsubstituted or substituted with hydroxy, 1-6 fluoro or NR¹²R¹³, where R¹² and R³ are independently selected from hydrogen and C₁₋₆alkyl, (c) —C₃₋₆cycloalkyl, which is unsubstituted or substituted with hydroxy, 1-6 fluoro or —NR¹²R¹³, (d) benzyl, (e) phenyl, and (7) —CONR¹⁰R¹¹; R2 is selected from the group consisting of: (1) phenyl, which is substituted with R^{2a}, R^{2b} and R^{2c}, (2) C₁₋₈alkyl, which is unsubstituted or substituted with 1-6 halogen, hydroxy, —NR¹⁰R¹¹, phenyl or heterocycle, where the phenyl or heterocycle is substituted with R^{2a}, R^{2b} and R^{2c}, (3) C₃₋₆cycloalkyl, which is unsubstituted or substituted with 1-6 halogen, hydroxy or —NR¹⁰R¹¹, and (4) —C₁₋₆alkyl-(C₃₋₆cycloalkyl), which is unsubstituted or substituted with 1-6 halogen, hydroxy or —NR¹⁰R¹¹; R^{2a}, R^{2b} and R^{2c} are independently selected from the group consisting of: (1) hydrogen, (2) halogen, (3) —C₁₋₆alkyl, which is unsubstituted or substituted with: (a) 1-6 halogen, (b) phenyl, (c) C₁₋₆cycloalkyl, or (d) —NR¹⁰R¹¹, (4) —O—C₁₋₆alkyl, which is unsubstituted or substituted with 1-6 halogen, (5) hydroxy, (6) —SCF₃, (7) —SCHF₂, (8) —SCH₃, (9) —CO₂R⁹, (10) —CN, (11) —SO₂R⁹, (12) —SO₂—NR¹⁰R¹¹, (13) —NR¹⁰R¹¹, (14) —CONR¹⁰R¹¹, and (15) —NO₂; R³ is selected from the group consisting of: (1) C₁₋₆alkyl, which is unsubstituted or substituted with 1-6 halogen, hydroxyl, or —NR¹⁰R¹¹, (2) C₃₋₆cycloalkyl, which is unsubstituted or substituted with 1-6 halogen, hydroxyl or NR¹⁰R¹¹, R⁴ and R⁵ are independently selected from the group consisting of: (1) hydrogen, and (2) C₁₋₆alkyl, which is unsubstituted or substituted with halogen or hydroxyl, or R⁴ and R⁵ taken together form a C₃₋₆cycloalkyl ring; A is selected from the group consisting of: (1) —O—, and (2) —NR¹⁰—; m is zero or one, whereby when m is zero R² is attached directly to the carbonyl; and pharmaceutically acceptable salts thereof and individual enantiomers and diastereomers thereof, or a pharmaceutically acceptable salt thereof, or a prodrug of the compound or its pharmaceutically acceptable salt. In certain such embodiments, the GlyT1 inhibitor is a compound having a formula of

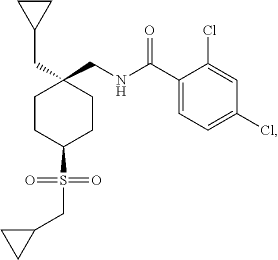 [SMILES: O=C(NC[C@]1(CC2CC2)CC[C@H](S(=O)(=O)CC2CC2)CC1)c1ccc(Cl)cc1Cl]

or a pharmaceutically acceptable salt thereof, or a prodrug of the compound or its pharmaceutically acceptable salt.

In certain embodiments, the GlyT1 inhibitor is a compound of Formula VIII,

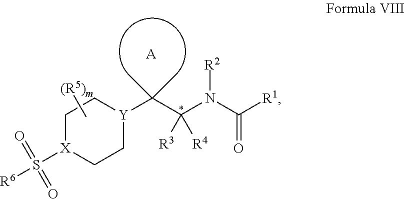

wherein R¹ is phenyl independently substituted from 1 to 5 times with halogen, C₁-C₃ alkyl, C₃-C₆ cycloalkyl, OR⁹, or SR¹⁰, wherein C₁-C₃ alkyl and C₃-C₆ cycloalkyl are optionally substituted with 1 to 10 times with R⁷; R² is H; R³ and R₄ are each individually H or CH₃; R⁵ is selected from the group consisting of: (1) hydrogen, (2) C₁-C₆ alkyl which is optionally substituted from 1 to 11 times with R⁷, (3) gem-dialkyl, and (4) gem-dihalo; or two R⁵ substituents on the same carbon, together with the carbon atom to which they are attached, may form a 3-, 4-, or 5-membered cycloalkyl optionally substituted from 1 to 10 times with R⁷; or two R⁵ substituents on adjacent carbons of the ring to which they are attached, together may form a 3-, 4-, 5- or 6-membered cycloalkyl optionally substituted from 1 to 10 times with R⁷; R⁶ is

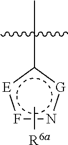

wherein E, F, and G are each independently nitrogen or carbon and R^{6a} is C₁-C₂ alkyl, which is optionally substituted 1 to 5 times with halogen or deuterium; R⁷ is selected from the group consisting of: (1) hydrogen, (2) halogen, (3) deuterium, (4) gem-dialkyl, (5) gem-dihalo, (6) —OR⁹, —NR¹¹R¹², —NR¹¹C(O)ₚR¹⁰, —S(O)ₚR¹⁰, —CN, —NO2, —C(O)ₚR¹⁰, —C(O)NR¹¹R¹², or —NR¹¹C(S)R¹⁰, and (7) oxo or thio; R⁸ is selected from the group consisting of: (1) hydrogen, (2) halogen, (3) C₁-C₆ alkyl, C₂-C₆ alkenyl, C₂-C₆ alkynyl, C₃-C₇ cycloalkyl, or C₄-C₇ cycloalkylalkyl, wherein each of the C₁-C₆ alkyl, C₂-C₆ alkenyl, C₂-C₆ alkynyl, C₃-C₇ cycloalkyl, and C₄-C₇ cycloalkylalkyl is independently and optionally substituted from 1 to 11 times with R⁷, or (4) —OR⁹, —NR¹¹R¹², —NR¹¹C(O)ₚR¹⁰, —S(O)ₚR¹⁰, —CN, —NO2, —C(O)ₚR¹⁰, —C(O)NR¹¹R¹², or —NR¹¹C(S)R¹⁰; R⁹ is selected from the group consisting of hydrogen, C₁-C₄ alkyl, C₃-C₇ cycloalkyl, C₄-C₇ cycloalkylalkyl, —C(O)NR¹¹R¹², and —C(O)ₚR¹⁰, wherein each of C₁-C₄ alkyl, C₃-C₇ cycloalkyl, and C₄-C₇ cycloalkylalkyl is optionally substituted from 1 to 11 times with R⁷; R¹⁰ is selected from the group consisting of hydrogen, C₁-C₄ alkyl, C₃-C₇ cycloalkyl C₄-C₇ cycloalkylalkyl, aryl, and heteroaryl, wherein each of C₁-C₄ alkyl, C₃-C₇ cycloalkyl, and C₄-C₇ cycloalkylalkyl is optionally substituted from 1 to 11 times with substituents as defined in R₇ and aryl or heteroaryl is optionally substituted from 1 to 10 times with R⁸; R¹¹ and R¹² are each independently selected from the group consisting hydrogen, C₁-C₄ alkyl, C₃-C₇ cycloalkyl, C₄-C₇ cycloalkylalkyl, aryl, and heteroaryl, wherein each of C₁-C₄ alkyl, C₃-C₇ cycloalkyl, and C₄-C₇ cycloalkylalkyl is optionally substituted from 1 to 11 times with substituents as defined in R⁷ and aryl or heteroaryl is optionally substituted from 1 to 10 times with R⁸, or R¹¹ and R¹² are taken together with the nitrogen to which they are attached to form a saturated or partially saturated monocyclic or fused bicyclic heterocycle optionally substituted from 1 to 11 times with R⁷; A is

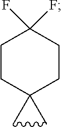

X is N; Y is N; p is 1, or 2; and m is 0; with the following provisos that: R⁶ cannot be (a) 1H-1,2,3-triazol-4-yl, or (b) 5-methylisoxazol-4-yl; or an oxide thereof, a pharmaceutically acceptable salt of the compound or its oxide, or an individual enantiomer or diastereomer thereof.

In certain embodiments, the GlyT1 inhibitor is a compound selected from any of the following:

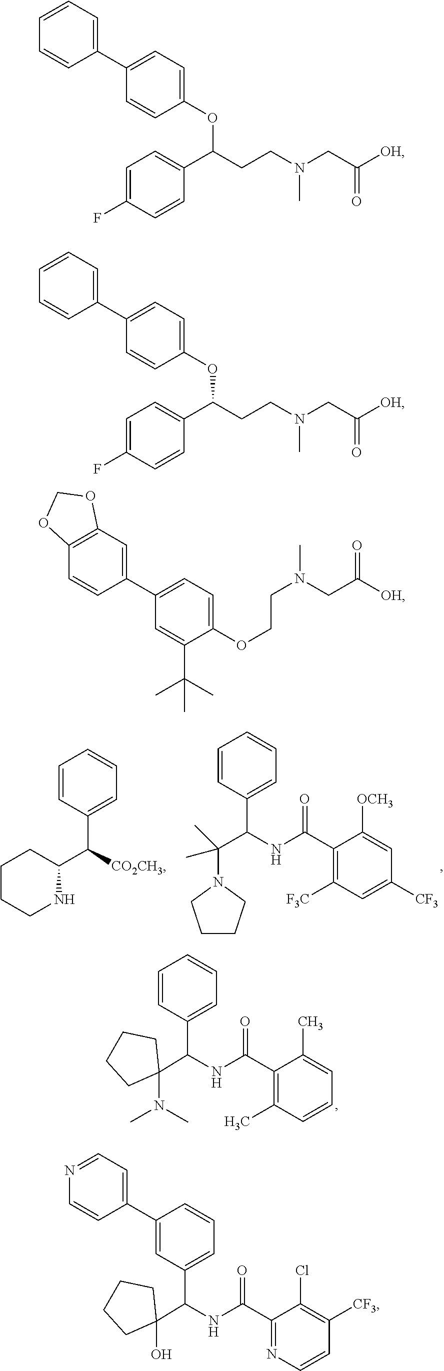

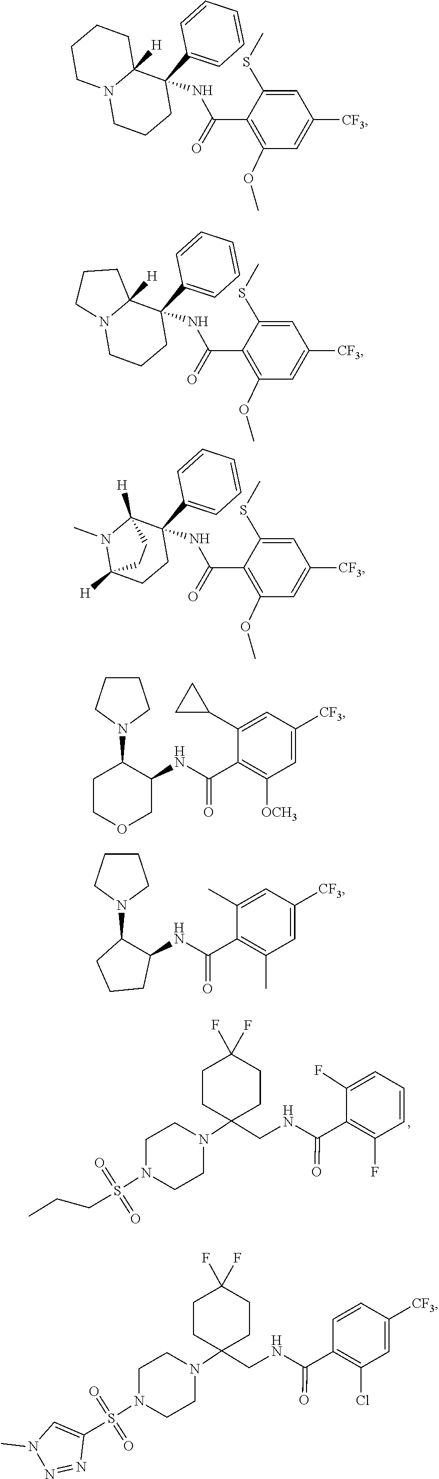

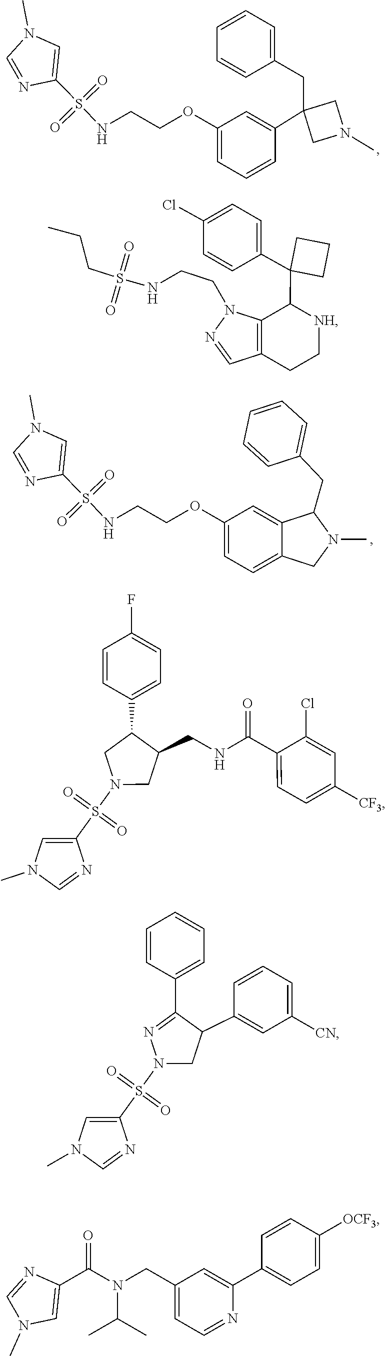

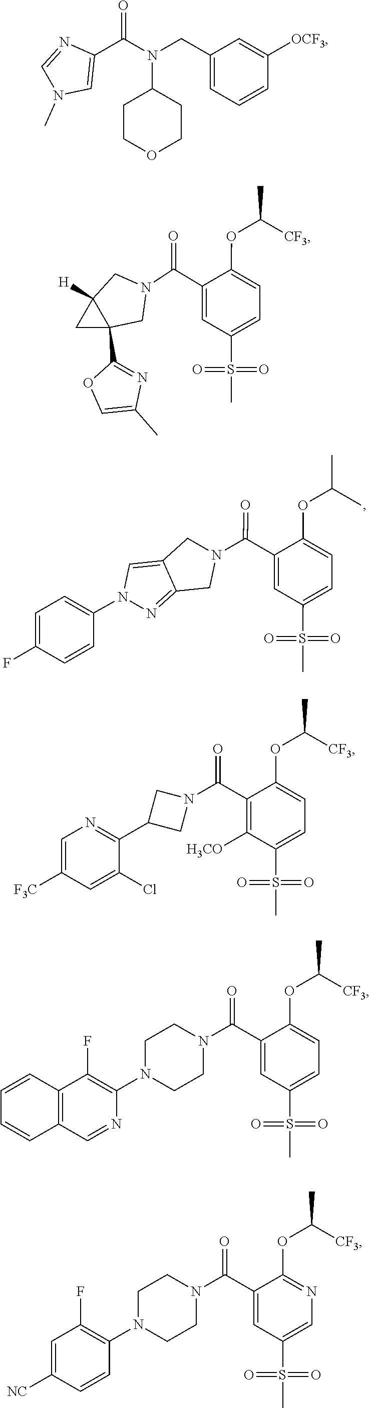

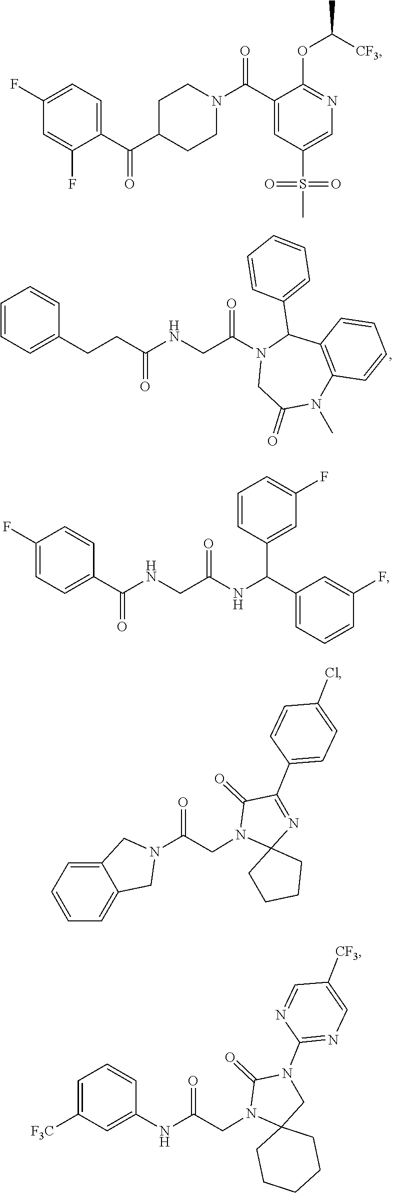

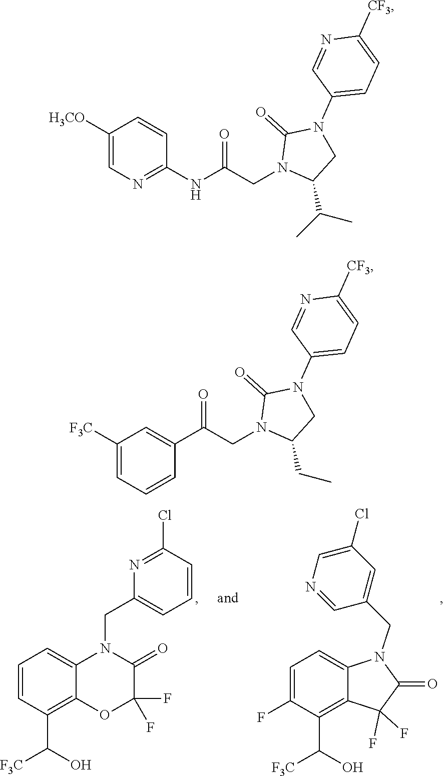

or a pharmaceutically acceptable salt thereof, or a prodrug of the compound or its pharmaceutically acceptable salt.

In certain embodiments, the GlyT1 inhibitor is a compound of formula IX,

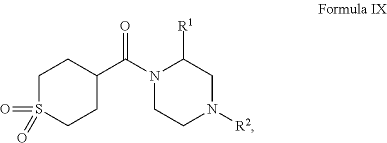

wherein R¹ represents phenyl or a 5 or 6 membered monocyclic heteroaryl having 1, 2, or 3 heteroatoms independently selected from O, N or S, wherein the phenyl or the heteroaryl is optionally substituted with one or more R³; R² represents aryl, a 5 or 6 membered monocyclic heteroaryl or a 8 to 10 membered bicyclic heteroaryl, the mono- or bicyclic heteroaryl having 1, 2, or 3 heteroatoms independently selected from O, N or S, wherein the aryl or the heteroaryl is optionally substituted with one or more R⁴; R³ is a halogen, a C₁₋₄-alkyl or a C₃₋₆-cycloalkyl, wherein the C₁₋₄-alkyl or the C₃₋₆-cycloalkyl is optionally substituted with one or more halogens; and R⁴ is a halogen, —CN, C₁₋₄-alkyl, C₃₋₆-cycloalkyl, —C₁₋₃-alkyl —C₃₋₆-cycloalkyl or —O—C₁₋₆ alkyl, wherein the C₁₋₄-alkyl, C₃₋₆-cycloalkyl, —C₁₋₃-alkyl —C₃₋₆-cycloalkyl or the —O—C₁₋₆-alkyl is optionally substituted with one or more halogens; or a pharmaceutically acceptable salt thereof, or a tautomer or stereoisomer of the compound or its pharmaceutically acceptable salt, or a mixture of any of the foregoing.

In certain embodiments, the GlyT1 inhibitor is a compound of formula X,

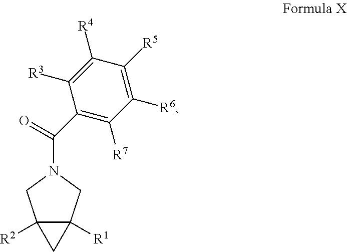

wherein R¹ is selected from the group consisting of a) 5 or 6 membered monocyclic heteroaryl, having 1, 2, 3 or 4 heteroatoms independently selected from the group consisting of O, N and S(O)r, b) 5 or 6 membered monocyclic partially saturated heterocycloalkyl, having 1, 2 or 3 heteroatoms independently selected from the group consisting of O, N and S(O)r, and c) 9 or 10 membered bicyclic heteroaryl, having 1, 2 or 3 heteroatoms independently selected from the group consisting of O, N and S(O)ᵣ, wherein r is 0, 1 or 2; wherein each of said groups a), b) and c) is optionally substituted with 1 or more substituents independently selected from the group consisting of C₁₋₄-alkyl-, C₁₋₄-alkyl-O—, oxetanyl, tetrahydrofuranyl, tetrahydropyranyl, C₃₋₆-cycloalkyl- and C₃₋₆-cycloalkyl-O— and in case a substituent is attached to a nitrogen ring atom said substituent is selected from the group consisting of C₁₋₄-alkyl-, C₁₋₄-alkyl-CO—, C₃₋₆-cycloalkyl- and C₃₋₆-cycloalkyl-CO—, and wherein each of said C₁₋₄-alkyl-, C₁₋₄-alkyl-O—, C₁₋₄-alkyl-CO—, oxetanyl, tetrahydrofuranyl, tetrahydropyranyl, C₃₋₆-cycloalkyl-, C₃₋₆-cycloalkyl-CO— or C₃₋₆-cycloalkyl-O— substituents may be substituted by 1 or more substituents independently selected from the group consisting of fluoro, —CF₃, —CHF₂, —CH₂F and —CN; R² is selected from the group consisting of hydrogen, C₁₋₄-alkyl-, C₁₋₄-alkyl-O—, —CN and C₃₋₆-cycloalkyl-, wherein each of said C₁₋₄-alkyl-, C₁₋₄-alkyl-O— and C₃₋₆-cycloalkyl-group may be optionally substituted with 1, 2, 3 or more substituents independently selected from the group consisting of fluoro, —CF₃, —CHF₂, —CH₂F and —CN; R³ is selected from the group consisting of C₁₋₆-alkyl-O—, C₃₋₆-cycloalkyl-O—, morpholino, pyrazolyl and a 4 to 7 membered, monocyclic heterocycloalkyl-O— with 1 oxygen atom as ring member and optionally 1 or 2 heteroatoms independently selected from the group consisting of 0, N and S(O)ₛ with s=0, 1 or 2, wherein said C₁₋₆-alkyl-O— and said C₃₋₆-cycloalkyl-O— may be optionally substituted with 1, 2, 3 or more substituents independently selected from the group consisting of fluoro, —CF₃, —CHF₂, —CH₂F, —CN, C₁₋₄-alkyl-, C₃₋₆-cycloalkyl-, C₁₋₆-alkyl-O— and C₃₋₆-cycloalkyl-O—; R⁴ is hydrogen; or R³ and R⁴ together with the ring atoms of the phenyl group to which they are bound may form a 4, 5 or 6 membered, monocyclic, partially saturated heterocycloalkyl or a heteroaryl each of which having 1, 2 or 3 heteroatoms independently selected from the group consisting of O, N and S(O)ₛ with s=0, 1 or 2, wherein there must be 1 ring oxygen atom that is directly attached to the ring carbon atom of said phenyl group to which R³ is attached to in general formula (I); wherein said heterocycloalkyl group may be optionally substituted with 1, 2, 3 or more substituents independently selected from the group consisting of fluoro, —CF₃, —CHF₂, —CH₂F, —CN, C₁₋₄-alkyl-, C₃₋₆-cycloalkyl-, C₁₋₆-alkyl-O—, C₃₋₆-cycloalkyl-O—, oxetanyl-O—, tetrahydrofuranyl-O and tetrahydropyranyl-O—; R⁵ is hydrogen; R⁶ is selected from the group consisting of hydrogen, C₁₋₄-alkyl-SO₂—, C₃₋₆-cycloalkyl-SO₂ and —CN; R⁷ is hydrogen; or one of the pairs a) R⁶ and R⁷ or b) R⁶ and R⁵ form together with the ring atoms of the phenyl group to which they are bound, a 5 or 6 membered, partially saturated monocyclic heterocycloalkyl group having 1, 2 or 3 heteroatoms independently selected from the group consisting of O, N and S(O)ᵤ with u=0, 1 or 2, wherein there must be 1 —SO₂— member that is directly attached to the ring carbon atom of said phenyl group to which R⁶ is attached to in general formula (I), wherein said heterocycloalkyl group may be optionally substituted with 1, 2, 3 or more substituents independently selected from the group consisting of fluoro, —CF₃, —CHF₂, —CH₂F, —CN, C₁₋₄-alkyl-, C₁₋₆-alkyl-O— and C₃₋₆-cycloalkyl-O— or a pharmaceutically acceptable salt thereof. In certain such embodiments, the GlyT1 inhibitor is a compound having a formula

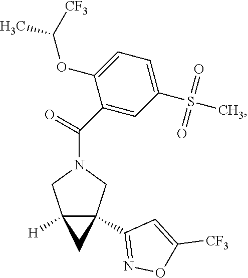 [SMILES: C[C@@H](Oc1ccc(S(C)(=O)=O)cc1C(=O)N1C[C@@H]2C[C@]2(c2cc(C(F)(F)F)on2)C1)C(F)(F)F]

or a pharmaceutically acceptable salt thereof.

In some embodiments, the GlyT1 inhibitor is a compound of Formula XI,

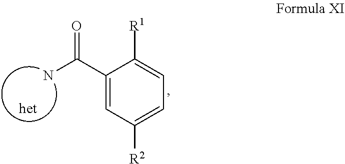

wherein R¹ is halogen, —OR^{1′}, —SR^{1″}, cycloalkyl, cyclic amide, heterocycloalkyl, aryl or 5- or 6-membered heteroaryl containing one, two or three heteroatoms selected from the group consisting of oxygen, sulphur and nitrogen; R^{1′} and R^{1″} are each independently hydrogen, lower alkyl, lower alkyl substituted by halogen, —(CH₂)ₓ-cycloalkyl or —(CH₂)ₓ-aryl; R² is —S(O)₂-lower alkyl, —S(O)₂NH-lower alkyl, NO₂ or CN;

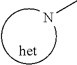

is an aromatic or partially aromatic bicyclic amine, having one or two additional N-atoms selected from the group consisting of

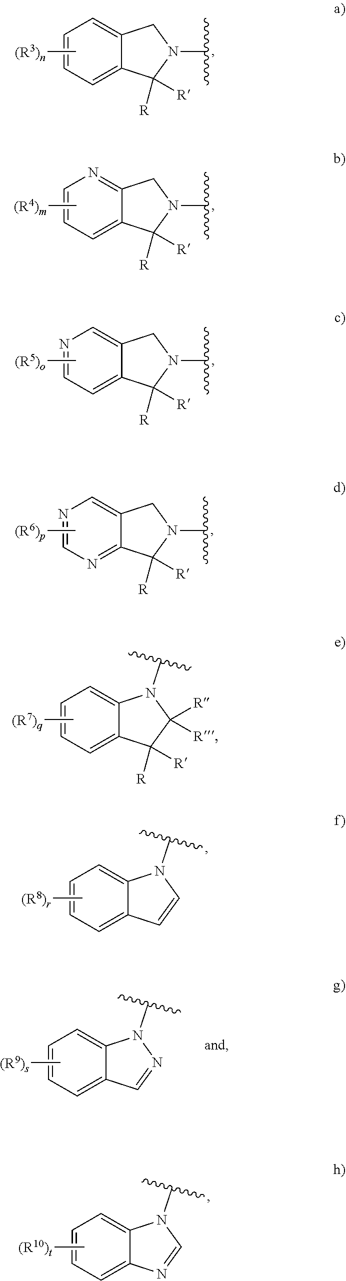

and wherein one of the additional N-ring atoms of the aromatic or partially aromatic bicyclic amine can be available in form of its oxide

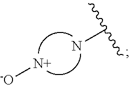

R3 to R10 are each independently hydrogen, hydroxy, halogen, ═O, lower alkyl, cycloalkyl, heterocycloalkyl, lower alkoxy, CN, NO2, NH2, aryl, 5- or 6-membered heteroaryl containing one, two or three heteroatoms selected from the group consisting of oxygen, sulphur and nitrogen, —NH-lower alkyl, —N(lower alkyl)2, cyclic amide, —C(O)-cyclic amide, S-lower alkyl, —S(O)2-lower alkyl, lower alkyl substituted by halogen, lower alkoxy substituted by halogen, lower alkyl substituted by hydroxy, —O—(CH2)y-lower alkoxy, —O(CH2)yC(O)N(lower alkyl)2, —C(O)-lower alkyl, —O—(CH2)x-aryl, —O—(CH2)x-cycloalkyl, —O—(CH2)x-heterocycloalkyl, —C(O)O-lower alkyl, —C(O)—NH-lower alkyl, —C(O)—N(lower alkyl)2, 2-oxy-5-aza-bicyclo[2.2.1]hept-5-yl or 3-oxa-8-aza-bicyclo[3.2.1]oct-8-yl; R, R′, R″ and R′″ are each independently hydrogen or lower alkyl; or R′ and R′″ in group e) together with —(CH2)4- form a six membered ring; and wherein all aryl-, cycloalkyl-, cyclic amide, heterocycloalkyl- or 5 or 6 membered heteroaryl groups as defined for R1, R1′, R1″ and R3 to R10 are unsubstituted or substituted by one or more substituents selected from the group consisting of hydroxy, ═O, halogen, lower alkyl, phenyl, lower alkyl substituted by halogen and lower alkoxy; n, m, o, p, q, r, s and t are each independently 1 or 2; x is 0, 1 or 2; and y is 1 or 2; or a pharmaceutically acceptable acid addition salt thereof.

In certain embodiments, the pharmaceutical composition further comprises a pharmaceutically acceptable carrier.

In certain embodiments, the subject is a subject in need thereof.

In certain embodiments, the GlyT1 inhibitor, or pharmaceutically acceptable salt thereof, or prodrug of the GlyT1 inhibitor or its pharmaceutically acceptable salt, is administered in a therapeutically effective amount.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

FIG. 1A and FIG. 1B show the RPS19 mRNA levels in a TF-1 cell line transduced with various lentiviruses encoding shRNAs targeting RPS19 or a control scrambled shRNA. The shRNA expression was inducible with doxycycline treatment. FIG. 1A shows the RPS19 mRNA expression in TF-1 cells transduced with RPS19-shRNA #a (TF-1/shRNA #a), RPS19-shRNA #b (TF-1/shRNA #b), or scrambled shRNA (TF-1/scrambled) after the cells were treated for 2 days with doxycycline. FIG. 1B shows the RPS19 mRNA expression in TF-1/shRNA #a, TF-1/shRNA #b and TF-1/scrambled cells after the cells were treated for 4 days with doxycycline.

FIG. 2A and FIG. 2B show the RPS19 protein levels in a TF-1 cell line transduced with a lentiviruses encoding the shRNAs targeting RPS19 or a control scrambled shRNA. The shRNA expression was inducible with doxycycline treatment. FIG. 2A shows a western blot of the RPS19 protein levels in TF-1/shRNA #a, TF-1/shRNA #b and TF-1/scrambled cells after 4 days of treatment with doxycycline. FIG. 2B shows a quantification of the western blot described in FIG. 2A.

FIG. 3A and FIG. 3B show the proliferation of TF-1 cells transduced with various lentiviruses encoding shRNAs targeting RPS19 or a control scrambled shRNA, wherein the cell line was treated with either erythropoietin (EPO) or granulocyte-macrophage colony-stimulating factor (GMCSF) for 6 days. FIG. 3A shows the cell number in TF-1/shRNA #a, TF-1/shRNA #b, or TF-1/scrambled after the cells were treated for 6 days with EPO. FIG. 3B shows the cell number in TF-1/shRNA #a, TF-1/shRNA #b and TF-1/scrambled after the cells were treated for 6 days with GMCSF.

FIG. 4A and FIG. 4B show the cell viability measured using CellTiter-Glo® (CTG) of TF-1 cells transduced with various lentiviruses encoding shRNAs targeting RPS19 or a control scrambled shRNA, wherein the cell line was treated with either erythropoietin (EPO) or granulocyte-macrophage colony-stimulating factor (GMCSF) for 6 days. FIG. 4A shows the cell viability of TF-1 cells transduced with either RPS19-shRNA #a, RPS19-shRNA #b, or scramble shRNA after the cells were treated for 6 days with EPO. FIG. 4B shows the cell viability of TF-1 cells transduced with either RPS19-shRNA #a, RPS19-shRNA #b, or scramble shRNA after the cells were treated for 6 days with GMCSF.

FIG. 5 shows that bitopertin treatment in TF-1 cells with RPS19 knockdown reverses the anti-proliferative effects caused by RPS19 knockdown. Prior to treatment with bitopertin, the TF-1/shRNA #a cells were treated with doxycycline for 4 days to induce RPS19 knockdown by shRNA #a. TF-1/scramble shRNA cells were treated similarly as an experimental control. On day 4, both TF-1/shRNA #a and TF-1/scramble shRNA cells were seeded to the 12-well cell culture plates at a density of 1×10⁵ cells per well. Bitopertin was added to the cells in 12-well plates for 48 hours of treatment from day 4 to day 6. FIG. 5 shows the cell number of TF-1 cells transduced with either RPS19-shRNA #a or scramble shRNA after the cells were treated for 2 days with either DMSO, 4 nM bitopertin, or 37 nM bitopertin. Each of the TF-1 cells were also treated with doxycycline and GMCSF, which induces shRNA expression and stimulates proliferation, for the entirety of the experiment.

FIG. 6 shows the cell viability measured using CellTiter-Glo® (CTG) of TF-1 cells transduced with a lentivirus encoding either a shRNA targeting RPS19 or a control scrambled shRNA. The cells were treated with (1) doxycycline during the entire cell culture period to induce shRNA expression; (2) GMCSF during the entire 6 days of cell culture period to induce proliferation. At day 4 of cell culture, the TF-1 cells were seeded to the 96-well cell culture plates at a density of 1×104 cells per well, and were treated with varying doses of bitopertin for two days of the culture period, from day 4 to day 6.

### DETAILED DESCRIPTION OF THE APPLICATION

Unless defined otherwise, all technical and scientific terms have the same meaning as is commonly understood by one of ordinary skill in the art to which the embodiments disclosed belongs.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

The term “acyl” is art-recognized and refers to a group represented by the general formula hydrocarbylC(O)—, preferably alkylC(O)—.

As used herein, the term “acylamino” means an amino group substituted by an acyl group (e.g., —O—C(═O)—H or —O—C(═O)-alkyl). An example of an acylamino is —NHC(═O)H or —NHC(═O)CH₃. The term “lower acylamino” refers to an amino group substituted by a lower acyl group (e.g., —O—C(═O)—H or —O—C(═O)—C₁₋₆alkyl). An example of a lower acylamino is —NHC(═O)H or —NHC(═O)CH₃.

The term “acyloxy” is art-recognized and refers to a group represented by the general formula hydrocarbylC(O)O—, preferably alkylC(O)O—.

As used herein, the term “alkenyl” means a straight or branched alkyl group having one or more double carbon-carbon bonds and 2-20 carbon atoms, including, but not limited to, ethenyl, 1-propenyl, 2-propenyl, 2-methyl-1-propenyl, 1-butenyl, 2-butenyl, and the like. In some embodiments, the alkenyl chain is from 2 to 10 carbon atoms in length, from 2 to 8 carbon atoms in length, from 2 to 6 carbon atoms in length, or from 2 to 4 carbon atoms in length.

The terms “alkoxy”, “phenyloxy”, “benzoxy” and “pyrimidinyloxy” refer to an alkyl group, phenyl group, benzyl group, or pyrimidinyl group, respectively, each optionally substituted, that is bonded through an oxygen atom. For example, the term “alkoxy” means a straight or branched —O-alkyl group of 1 to 20 carbon atoms, including, but not limited to, methoxy, ethoxy, n-propoxy, isopropoxy, t-butoxy, and the like. In some embodiments, the alkoxy chain is from 1 to 10 carbon atoms in length, from 1 to 8 carbon atoms in length, from 1 to 6 carbon atoms in length, from 1 to 4 carbon atoms in length, from 2 to 10 carbon atoms in length, from 2 to 8 carbon atoms in length, from 2 to 6 carbon atoms in length, or from 2 to 4 carbon atoms in length.

As used herein, the term “alkyl” means a saturated hydrocarbon group which is straight-chained or branched. An alkyl group can contain from 1 to 20, from 2 to 20, from 1 to 10, from 2 to 10, from 1 to 8, from 2 to 8, from 1 to 6, from 2 to 6, from 1 to 4, from 2 to 4, from 1 to 3, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4-dimethylpentyl, octyl, 2,2,4-trimethylpentyl, nonyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the term “alkylamino” means an amino group substituted by an alkyl group having from 1 to 6 carbon atoms. An example of an alkylamino is —NHCH₂CH₃.

As used herein, the term “alkylene” or “alkylenyl” means a divalent alkyl linking group. An example of an alkylene (or alkylenyl) is methylene or methylenyl (—CH₂—).

As used herein, the term “alkylthio” means an —S-alkyl group having from 1 to 6 carbon atoms. An example of an alkylthio group is —SCH₂CH₃.

As used herein, the term “alkynyl” means a straight or branched alkyl group having one or more triple carbon-carbon bonds and 2-20 carbon atoms, including, but not limited to, acetylene, 1-propylene, 2-propylene, and the like. In some embodiments, the alkynyl chain is 2 to 10 carbon atoms in length, from 2 to 8 carbon atoms in length, from 2 to 6 carbon atoms in length, or from 2 to 4 carbon atoms in length.

The term “amide”, as used herein, refers to a group

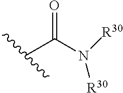

wherein each R³⁰ independently represent a hydrogen or hydrocarbyl group, or two R³⁰ are taken together with the N atom to which they are attached complete a heterocycle having from 4 to 8 atoms in the ring structure.

As used herein, the term “amidino” means —C(═NH)NH₂.

The terms “amine” and “amino” are art-recognized and refer to both unsubstituted and substituted amines and salts thereof, e.g., a moiety that can be represented by

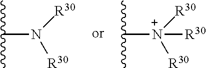

wherein each R³⁰ independently represents a hydrogen or a hydrocarbyl group, or two R³⁰ are taken together with the N atom to which they are attached complete a heterocycle having from 4 to 8 atoms in the ring structure.

As used herein, the term “aminoalkoxy” means an alkoxy group substituted by an amino group. An example of an aminoalkoxy is —OCH₂CH₂NH₂.

As used herein, the term “aminoalkyl” means an alkyl group substituted by an amino group. An example of an aminoalkyl is —CH₂CH₂NH₂.

As used herein, the term “aminosulfonyl” means —S(═O)₂NH₂.

As used herein, the term “aminoalkylthio” means an alkylthio group substituted by an amino group. An example of an aminoalkylthio is —SCH₂CH₂NH₂.

As used herein, the term “amphiphilic” means a three-dimensional structure having discrete hydrophobic and hydrophilic regions. An amphiphilic compound suitably has the presence of both hydrophobic and hydrophilic elements.

As used herein, the term “animal” includes, but is not limited to, humans and non-human vertebrates such as wild, domestic, and farm animals.

As used herein, the term “aryl” means a monocyclic, bicyclic, or polycyclic (e.g., having 2, 3 or 4 fused rings) aromatic hydrocarbons. In some embodiments, aryl groups have from 6 to 20 carbon atoms or from 6 to 10 carbon atoms. Examples of aryl groups include, but are not limited to, phenyl, naphthyl, anthracenyl, phenanthrenyl, indanyl, indenyl, tetrahydronaphthyl, and the like. Examples of aryl groups include, but are not limited to:

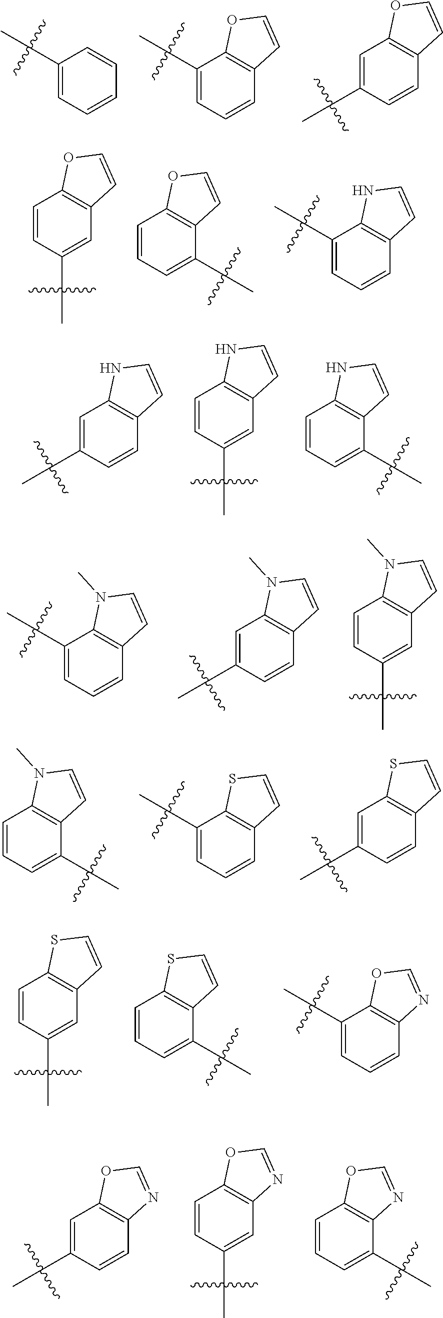

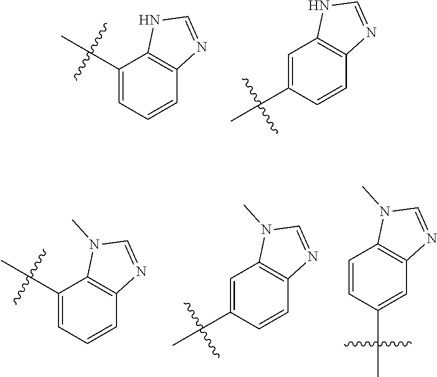

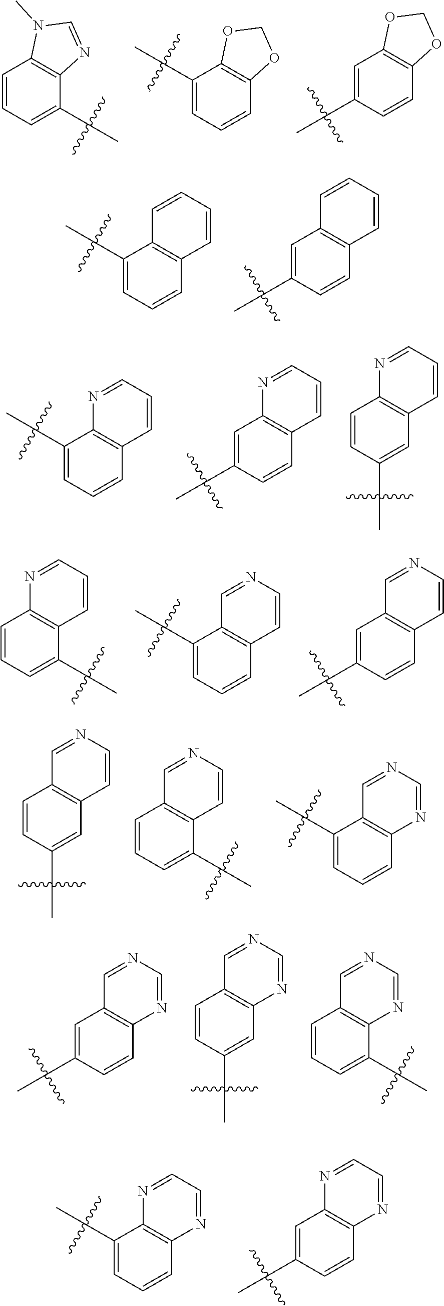

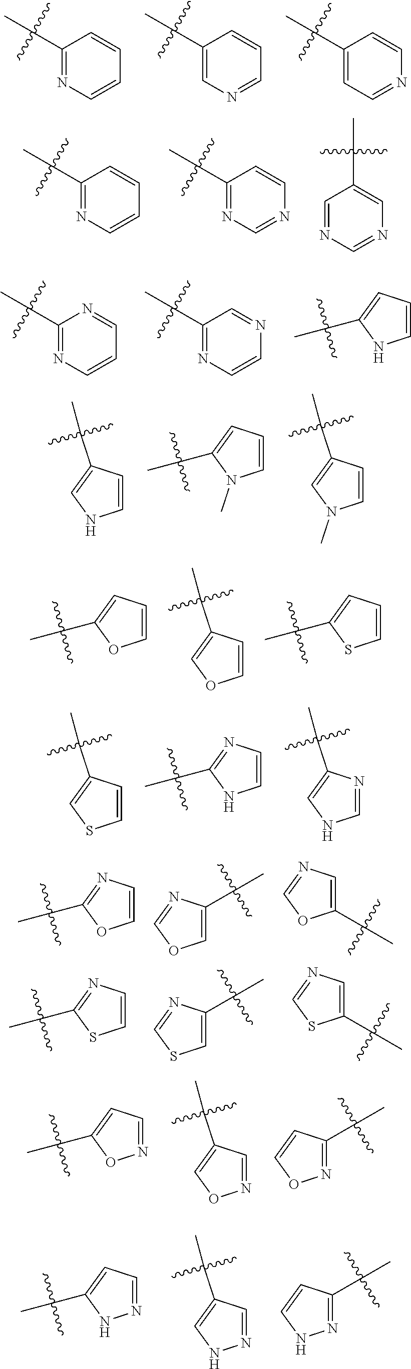

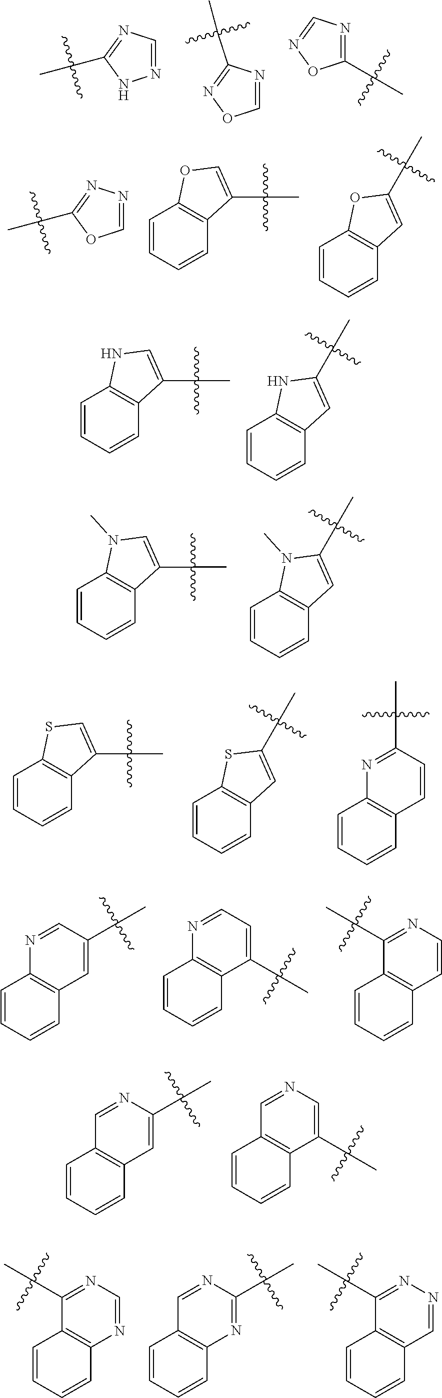

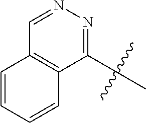 [SMILES: *c1nncc2ccccc12]

As used herein, the term “arylalkyl” means a C₁₋₆alkyl substituted by aryl.

As used herein, the term “arylamino” means an amino group substituted by an aryl group. An example of an arylamino is —NH(phenyl).

As used herein, the term “arylene” means an aryl linking group, i.e., an aryl group that links one group to another group in a molecule.

The term “carbamate” is art-recognized and refers to a group

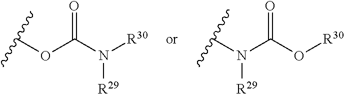

wherein R²⁹ and R³⁰ independently represent hydrogen or a hydrocarbyl group, such as an alkyl group, or R²⁹ and R³⁰ taken together with the intervening atom(s) complete a heterocycle having from 4 to 8 atoms in the ring structure.

As used herein, the term “carbamoyl” means —C(═O)—NH₂.

As used herein, the term “carbocycle” means a 5- or 6-membered, saturated or unsaturated cyclic ring, optionally containing O, S, or N atoms as part of the ring. Examples of carbocycles include, but are not limited to, cyclopentyl, cyclohexyl, cyclopenta-1,3-diene, phenyl, and any of the heterocycles recited above.

The term “carbocyclylalkyl”, as used herein, refers to an alkyl group substituted with a carbocycle group.

The term “carbonate” is art-recognized and refers to a group —OCO₂—R³⁰, wherein R³⁰ represents a hydrocarbyl group.

The term “carboxy”, as used herein, refers to a group represented by the formula —CO₂H.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

As used herein, the term, “compound” means all stereoisomers, tautomers, and isotopes of the compounds described herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” means bringing together of two elements in an in vitro system or an in vivo system. For example, “contacting” a GlyT1 transporter inhibitor with a GlyT1 transporter with an individual or patient or cell includes the administration of the compound to an individual or patient, such as a human, as well as, for example, introducing a compound into a sample containing a cellular or purified preparation containing the GlyT1 transporter.

As used herein, the term “cyano” means —CN.

As used herein, the term “cycloalkyl” means non-aromatic cyclic hydrocarbons including cyclized alkyl, alkenyl, and alkynyl groups that contain up to 20 ring-forming carbon atoms. Cycloalkyl groups can include mono- or polycyclic ring systems such as fused ring systems, bridged ring systems, and spiro ring systems. In some embodiments, polycyclic ring systems include 2, 3, or 4 fused rings. A cycloalkyl group can contain from 3 to 15, from 3 to 10, from 3 to 8, from 3 to 6, from 4 to 6, from 3 to 5, or 5 or 6 ring-forming carbon atoms. Ring-forming carbon atoms of a cycloalkyl group can be optionally substituted by oxo or sulfido. Examples of cycloalkyl groups include, but are not limited to, cyclopropyl, cyclobutyl, cyclopentyl, cyclohexyl, cycloheptyl, cyclooctyl, cyclononyl, cyclopentenyl, cyclohexenyl, cyclohexadienyl, cycloheptatrienyl, norbornyl, norpinyl, norcamyl, adamantyl, and the like. Also included in the definition of cycloalkyl are moieties that have one or more aromatic rings fused (having a bond in common with) to the cycloalkyl ring, for example, benzo or thienyl derivatives of pentane, pentene, hexane, and the like (e.g., 2,3-dihydro-1H-indene-1-yl, or 1H-inden-2(3H)-one-1-yl).

As used herein, the term “cycloalkylalkyl” means a C₁₋₆alkyl substituted by cycloalkyl.

As used herein, the term “dialkylamino” means an amino group substituted by two alkyl groups, each having from 1 to 6 carbon atoms.

As used herein, the term “diazamino” means —N(NH₂)₂.

The term “ester”, as used herein, refers to a group —C(O)OR³⁰ wherein R³⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

As used herein, the term “facially amphiphilic” or “facial amphiphilicity” means compounds with polar (hydrophilic) and nonpolar (hydrophobic) side chains that adopt conformation(s) leading to segregation of polar and nonpolar side chains to opposite faces or separate regions of the structure or molecule.

As used herein, the term “glycine transporter” or “GlyT” refers to membrane protein that facilitates the transport of glycine across the plasma membrane of a cell. Non-limiting examples of glycine transports include glycine transporter 1 (GlyT1) and glycine transporter 2 (GlyT2).

As used herein, the term “GlyT1” or “GlyT1 transporter” means sodium- and chloride-dependent glycine transporter 1, also known as glycine transporter 1, is a protein that in humans is encoded by the SLC6A9 gene (Kim K M, Kingsmore S F, Han H, Yang-Feng T L, Godinot N, Seldin M F, Caron M G, Giros B (June 1994). “Cloning of the human glycine transporter type 1: molecular and pharmacological characterization of novel isoform variants and chromosomal localization of the gene in the human and mouse genomes”. Mol Pharmacol. 45 (4): 608-17; Jones E M, Fernald A, Bell G I, Le Beau M M (November 1995). “Assignment of SLC6A9 to human chromosome band 1p33 by in situ hybridization”. Cytogenet Cell Genet. 71 (3): 211), which is hereby incorporated by reference in its entirety.

As used herein, the term “GlyT2” or “GlyT2 transporter” means sodium- and chloride-dependent glycine transporter 2, also known as glycine transporter 2, is a protein that in humans is encoded by the SLC6A5 gene (Morrow J A, Collie I T, Dunbar D R, Walker G B, Shahid M, Hill D R (November 1998). “Molecular cloning and functional expression of the human glycine transporter GlyT2 and chromosomal localisation of the gene in the human genome”. FEBS Lett. 439 (3): 334-40), which is hereby incorporated by reference in its entirety.

As used herein, the term “GlyT1 inhibitor” means a compound that inhibits or blocks the activity of GlyT1 transporter including compounds inhibiting the activity of any isoform of GlyT1. Non-limiting examples of GlyT1 inhibitors are provided herein. In some embodiments, the GlyT1 inhibitor is a specific GlyT1 inhibitor, which means that the inhibitor has an inhibitor activity that is greater for GlyT1 as compared to GlyT2. In some embodiments, the inhibitor inhibits GlyT1 as compared to GlyT2 with at least, or about, 10%, 20%, 30%, 40%, 50%, 60%, 70%, 80%, 90%, 95%, 96%, 97%, 98%, 99% selectivity. In some embodiments, the GlyT1 inhibitor inhibits GlyT1 but does not inhibit or significantly inhibit the activity of GlyT2. A GlyT1 inhibitor that does not significantly inhibit the activity of GlyT2 if it inhibits the activity of GlyT2 less than 5%, 4%, 3%, 2%, or 1%. The selectivity of GlyT1 inhibitor is determined based on the known assays in the art such as the assays described in the published journal article (B. N. Atkinson, S. C. Bell, M. De Vivo, L. R. Kowalski, S. M. Lechner, V. I. Ognyanov, C.-S. Tham, C. Tsai, J. Jia, D. Ashton and M. A. Klitenick, ALX 5407: A Potent, Selective Inhibitor of the hGlyT1 Glycine Transporter, Molecular Pharmacology December 2001, 60 (6) 1414-1420), which is incorporated by its entirety.

As used herein, the term “GlyT2 inhibitor” means a compound that inhibits or blocks the activity of GlyT2 transporter including compounds inhibiting the activity of any isoform of GlyT2. In some embodiments, the GlyT2 inhibitor is a non-specific inhibitor, which means that it can also inhibit or block the activity of GlyT1. In some embodiments, the GlyT2 inhibitor is a specific GlyT2 inhibitor, which means that the inhibitor has an inhibitor activity that is greater for GlyT2 as compared to GlyT1. In some embodiments, the inhibitor inhibits GlyT2 as compared to GlyT1 with at least, or about, 10%, 20%, 30%, 40%, 50%, 60%, 70%, 80%, 90%, 95%, 96%, 97%, 98%, 99% selectivity. In some embodiments, the GlyT2 inhibitor inhibits GlyT2 activity but does not inhibit or significantly inhibit the activity of GlyT1. A GlyT2 inhibitor that does not significantly inhibit the activity of GlyT1 if it inhibits the activity of GlyT1 less than 5%, 4%, 3%, 2%, or 1%. The selectivity of GlyT2 inhibitor is determined based on the known assays in the art such as the assays based described in the published journal article (B. N. Atkinson, S. C. Bell, M. De Vivo, L. R. Kowalski, S. M. Lechner, V. I. Ognyanov, C.-S. Tham, C. Tsai, J. Jia, D. Ashton and M. A. Klitenick, ALX 5407: A Potent, Selective Inhibitor of the hGlyT1 Glycine Transporter, Molecular Pharmacology December 2001, 60 (6) 1414-1420), which is incorporated by its entirety.

As used herein, the term “guanidino” means —NH(═NH)NH₂.

As used herein, the term “halo” means halogen groups including, but not limited to fluoro, chloro, bromo, and iodo.

As used herein, the term “haloalkoxy” means an —O-haloalkyl group. An example of an haloalkoxy group is OCF₃.

As used herein, the term “haloalkyl” means a C₁₋₆alkyl group having one or more halogen substituents. Examples of haloalkyl groups include, but are not limited to, CF₃, C₂F₅, CH₂F, CHF₂, CCl₃, CHCl₂, CH₂CF₃, and the like.

As used herein, the term “heteroaryl” means an aromatic heterocycle having up to 20 ring-forming atoms (e.g., C) and having at least one heteroatom ring member (ring-forming atom) such as sulfur, oxygen, or nitrogen. In some embodiments, the heteroaryl group has at least one or more heteroatom ring-forming atoms, each of which is, independently, sulfur, oxygen, or nitrogen. In some embodiments, the heteroaryl group has from 3 to 20 ring-forming atoms, from 3 to 10 ring-forming atoms, from 3 to 6 ring-forming atoms, or from 3 to 5 ring-forming atoms. In some embodiments, the heteroaryl group contains 2 to 14 carbon atoms, from 2 to 7 carbon atoms, or 5 or 6 carbon atoms. In some embodiments, the heteroaryl group has 1 to 4 heteroatoms, 1 to 3 heteroatoms, or 1 or 2 heteroatoms. Heteroaryl groups include monocyclic and polycyclic (e.g., having 2, 3 or 4 fused rings) systems. Examples of heteroaryl groups include, but are not limited to, pyridyl, pyrimidinyl, pyrazinyl, pyridazinyl, triazinyl, furyl, quinolyl, isoquinolyl, thienyl, imidazolyl, thiazolyl, indolyl (such as indol-3-yl), pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, pyranyl, oxadiazolyl, isoxazolyl, triazolyl, thianthrenyl, pyrazolyl, indolizinyl, isoindolyl, isobenzofuranyl, benzoxazolyl, xanthenyl, 2H-pyrrolyl, pyrrolyl, 3H-indolyl, 4H-quinolizinyl, phthalazinyl, naphthyridinyl, quinazolinyl, phenanthridinyl, acridinyl, perimidinyl, phenanthrolinyl, phenazinyl, isothiazolyl, phenothiazinyl, isoxazolyl, furanyl, phenoxazinyl groups, and the like. Suitable heteroaryl groups include 1,2,3-triazole, 1,2,4-triazole, 5-amino-1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, and 2-aminopyridine.

As used herein, the term “heteroarylalkyl” means a C₁₋₆alkyl group substituted by a heteroaryl group.

As used herein, the term “heteroarylamino” means an amino group substituted by a heteroaryl group. An example of a heteroarylamino is —NH-(2-pyridyl).

As used herein, the term “heteroarylene” means a heteroaryl linking group, i.e., a heteroaryl group that links one group to another group in a molecule.

The term “heteroatom” as used herein means an atom of any element other than carbon or hydrogen. Exemplary heteroatoms are nitrogen, oxygen, and sulfur.

As used herein, the term “heterocycle” or “heterocyclic ring” means a 5- to 7-membered mono- or bicyclic or 7- to 10-membered bicyclic heterocyclic ring system any ring of which may be saturated or unsaturated, and which consists of carbon atoms and from one to three heteroatoms chosen from N, O and S, and wherein the N and S heteroatoms may optionally be oxidized, and the N heteroatom may optionally be quaternized, and including any bicyclic group in which any of the above-defined heterocyclic rings is fused to a benzene ring. Particularly useful are rings containing one oxygen or sulfur, one to three nitrogen atoms, or one oxygen or sulfur combined with one or two nitrogen atoms. The heterocyclic ring may be attached at any heteroatom or carbon atom which results in the creation of a stable structure. Examples of heterocyclic groups include, but are not limited to, piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

As used herein, the term “heterocycloalkyl” means non-aromatic heterocycles having up to 20 ring-forming atoms including cyclized alkyl, alkenyl, and alkynyl groups, where one or more of the ring-forming carbon atoms is replaced by a heteroatom such as an O, N, or S atom. Hetercycloalkyl groups can be mono or polycyclic (e.g., fused, bridged, or spiro systems). In some embodiments, the heterocycloalkyl group has from 1 to 20 carbon atoms, or from 3 to 20 carbon atoms. In some embodiments, the heterocycloalkyl group contains 3 to 14 ring-forming atoms, 3 to 7 ring-forming atoms, or 5 or 6 ring-forming atoms. In some embodiments, the heterocycloalkyl group has 1 to 4 heteroatoms, 1 to 3 heteroatoms, or 1 or 2 heteroatoms. In some embodiments, the heterocycloalkyl group contains 0 to 3 double bonds. In some embodiments, the heterocycloalkyl group contains 0 to 2 triple bonds. Examples of heterocycloalkyl groups include, but are not limited to, morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, oxazolidinyl, isothiazolidinyl, pyrazolidinyl, thiazolidinyl, imidazolidinyl, pyrrolidin-2-one-3-yl, and the like. In addition, ring-forming carbon atoms and heteroatoms of a heterocycloalkyl group can be optionally substituted by oxo or sulfido. For example, a ring-forming S atom can be substituted by 1 or 2 oxo (form a S(O) or S(O)₂). For another example, a ring-forming C atom can be substituted by oxo (form carbonyl). Also included in the definition of heterocycloalkyl are moieties that have one or more aromatic rings fused (having a bond in common with) to the nonaromatic heterocyclic ring including, but not limited to, pyridinyl, thiophenyl, phthalimidyl, naphthalimidyl, and benzo derivatives of heterocycles such as indolene, isoindolene, 4,5,6,7-tetrahydrothieno[2,3-c]pyridine-5-yl, 5,6-dihydrothieno[2,3-c]pyridin-7(4H)-one-5-yl, isoindolin-1-one-3-yl, and 3,4-dihydroisoquinolin-1(2H)-one-3yl groups. Ring-forming carbon atoms and heteroatoms of the heterocycloalkyl group can be optionally substituted by oxo or sulfido.

As used herein, the term “heterocycloalkylalkyl” refers to a C₁₋₆alkyl substituted by heterocycloalkyl.

As used herein, the term “hydroxy” or “hydroxyl” means an —OH group.

As used herein, the term “hydroxyalkyl” or “hydroxylalkyl” means an alkyl group substituted by a hydroxyl group. Examples of a hydroxylalkyl include, but are not limited to, —CH₂OH and —CH₂CH₂OH.

As used herein, the term “individual” or “patient,” used interchangeably, means any animal, including mammals, such as mice, rats, other rodents, rabbits, dogs, cats, swine, cattle, sheep, horses, or primates, such as humans.

As used herein, the phrase “inhibiting activity,” such as enzymatic or transporter activity means reducing by any measurable amount the activity of an enzyme or transporter, such as the GlyT1 transporter.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein, the phrase “in situ gellable” means embracing not only liquids of low viscosity that form gels upon contact with the eye or with lacrimal fluid in the exterior of the eye, but also more viscous liquids such as semi-fluid and thixotropic gels that exhibit substantially increased viscosity or gel stiffness upon administration to the eye.

As used herein, the phrase “integer from X to Y” means any integer that includes the endpoints. For example, the phrase “integer from X to Y” means 1, 2, 3, 4, or 5.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “N-alkyl” refers to a alkyl chain that is substituted with an amine group. Non-limiting examples, include, but are not limited to

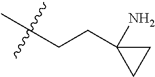 [SMILES: *CCC1(N)CC1]

and the like. The alkyl chain can be linear, branched, cyclic, or any combination thereof. In some embodiments, the alkyl comprises 1-10, 1-9, 1-8, 1-7, 1-6, 1-5, 1-4, 1-3, or 1-2 carbons.

As used herein, the term “nitro” means —NO₂.

As used herein, the term “n-membered”, where n is an integer, typically describes the number of ring-forming atoms in a moiety, where the number of ring-forming atoms is n. For example, pyridine is an example of a 6-membered heteroaryl ring and thiophene is an example of a 5-membered heteroaryl ring.

As used herein, the phrase “ophthalmically acceptable” means having no persistent detrimental effect on the treated eye or the functioning thereof, or on the general health of the subject being treated. However, it will be recognized that transient effects such as minor irritation or a “stinging” sensation are common with topical ophthalmic administration of drugs and the existence of such transient effects is not inconsistent with the composition, formulation, or ingredient (e.g., excipient) in question being “ophthalmically acceptable” as herein defined.

As used herein, the phrase “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent groups, provided that the normal valency of the designated atom or moiety is not exceeded, and that the substitution results in a stable compound. For example, if a methyl group is optionally substituted, then 3 hydrogen atoms on the carbon atom can be replaced with substituent groups.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

For a compound described herein that contains a basic group, such as an amine, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

For a compound described herein that contains an acidic group, such as a carboxylic acid group, base addition salts can be prepared by any suitable method available in the art, for example, treatment of such compound with a sufficient amount of the desired the desired base, either neat or in a suitable inert solvent. Examples of pharmaceutically acceptable base addition salts include, but are not limited to, lithium, sodium, potassium, calcium, ammonium, zinc, or magnesium salt, or other metal salts; organic amino salts, such as, alkyl, dialkyl, trialkyl, or tetra-alkyl ammonium salts.

Other examples of pharmaceutically acceptable salts include, but are not limited to, camsylate, sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present application.

As used herein, the term “phenyl” means —C₆H₅. A phenyl group can be unsubstituted or substituted with one, two, or three suitable substituents.

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

As used herein, the term “prodrug” means a derivative of a known direct acting drug, which derivative has enhanced delivery characteristics and therapeutic value as compared to the drug, and is transformed into the active drug by an enzymatic or chemical process. A common method for making a prodrug is to include one or more selected moieties which are hydrolyzed under physiologic conditions to yield the desired molecule. In certain embodiments, the prodrug is converted by an enzymatic activity of the host animal. For example, a prodrug with a nitro group on an aromatic ring could be reduced by reductase to generate the desired amino group of the corresponding active compound in vivo. In another example, functional groups such as a hydroxyl, carbonate, or carboxylic acid in the parent compound are presented as an ester, which could be cleaved by esterases. Additionally, amine groups in the parent compounds are presented in, but not limited to, carbamate, N-alkylated or N-acylated forms (Simplicio et al, “Prodrugs for Amines,” Molecules, (2008), 13:519-547). In certain embodiments, some or all of the compounds of described herein in a formulation represented above can be replaced with the corresponding suitable prodrug.

As used herein, the term “purified” means that when isolated, the isolate contains at least 90%, at least 95%, at least 98%, or at least 99% of a compound described herein by weight of the isolate.

As used herein, the phrase “quaternary ammonium salts” means derivatives of the disclosed compounds with one or more tertiary amine moieties wherein at least one of the tertiary amine moieties in the parent compound is modified by converting the tertiary amine moiety to a quaternary ammonium cation via alkylation (and the cations are balanced by anions such as Cl⁻, CH₃COO⁻, and CF₃COO⁻), for example methylation or ethylation.

As used herein, the term “ribosomal disorder” refers to any disease or malfunction of ribosomes. It can include a disease or a disorder linked to a mutated and/or abnormal function of a ribosome protein. It can also include a disease due to mutation in a ribosomal protein, or a disease due to a decreased level, or partial loss of function, of a ribosomal protein, or alternatively, a disease due to an increased level of a ribosomal protein, as compared to a normal healthy control subject. A disease or malfunction of ribosomes include, but are not limited to (i) diseases of ribosomal biogenesis proteins, (ii) diseases of small nucleolar ribonuceloproteins, and (iii) diseases of ribosomal proteins. Ribosomal disorders include, but are not limited to Diamond-Blackfan anemia, myelodysplastic syndrome associated (MDS) with isolated del(5q), Shwachman-Diamond syndrome, X-linked dyskeratosis congenital, and cartilage hair hypoplasia.

As used herein, the term “semicarbazone” means ═NNHC(═O)NH₂.

As used herein, the phrase “solubilizing agent” means agents that result in formation of a micellar solution or a true solution of the drug.

As used herein, the term “solution/suspension” means a liquid composition wherein a first portion of the active agent is present in solution and a second portion of the active agent is present in particulate form, in suspension in a liquid matrix.

As used herein, the phrase “substantially isolated” means a compound that is at least partially or substantially separated from the environment in which it is formed or detected.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this application, the heteroatoms such as nitrogen may have hydrogen substituents and/or any permissible substituents of organic compounds described herein which satisfy the valences of the heteroatoms.

Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

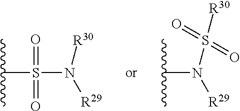

wherein R²⁹ and R³⁰ independently represents hydrogen or hydrocarbyl, such as alkyl, or R₂₉ and R³⁰ taken together with the intervening atom(s) complete a heterocycle having from 4 to 8 atoms in the ring structure.

The term “sulfoxide” is art-recognized and refers to the group —S(O)—R³⁰, wherein R³⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R³⁰, wherein R³⁰ represents a hydrocarbyl.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

The term “thioalkyl”, as used herein, refers to an alkyl group substituted with a thiol group.

The term “thioester”, as used herein, refers to a group —C(O)SR³⁰ or —SC(O)R³⁰ wherein R³⁰ represents a hydrocarbyl.

The term “thioether”, as used herein, is equivalent to an ether, wherein the oxygen is replaced with a sulfur.

As used herein, the terms “treat,” “treated,” or “treating” mean both therapeutic treatment and prophylactic measures wherein the object is to slow down (lessen) an undesired physiological condition, disorder or disease, or obtain beneficial or desired clinical results. Beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment. Thus, “treatment of anemia associated with a ribosomal disorder” or “treating anemia associated with a ribosomal disorder” means an activity that alleviates or ameliorates any of the primary phenomena or secondary symptoms associated with the anemia associated with a ribosomal disorder or other conditions described herein.

The term “urea” is art-recognized and may be represented by the general formula

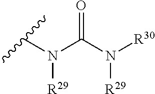

wherein R²⁹ and R³⁰ independently represent hydrogen or a hydrocarbyl, such as alkyl, or either occurrence of R²⁹ taken together with R³⁰ and the intervening atom(s) complete a heterocycle having from 4 to 8 atoms in the ring structure.

At various places in the present specification, substituents of compounds may be disclosed in groups or in ranges. It is specifically intended that embodiments include each and every individual subcombination of the members of such groups and ranges. For example, the term “C₁₋₆alkyl” is specifically intended to individually disclose methyl, ethyl, propyl, C₄alkyl, C₅alkyl, and C₆alkyl.

For compounds in which a variable appears more than once, each variable can be a different moiety selected from the Markush group defining the variable. For example, where a structure is described having two R groups that are simultaneously present on the same compound, the two R groups can represent different moieties selected from the Markush groups defined for R. In another example, when an optionally multiple substituent is designated in the form, for example,

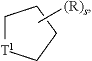

then it is understood that substituent R can occur s number of times on the ring, and R can be a different moiety at each occurrence. In the above example, where the variable T¹ is defined to include hydrogens, such as when T¹ is CH₂, NH, etc., any H can be replaced with a substituent.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

It is understood that the present embodiments encompasses the use, where applicable, of stereoisomers, diastereomers and optical stereoisomers of the compounds, as well as mixtures thereof. Additionally, it is understood that stereoisomers, diastereomers, and optical stereoisomers of the compounds, and mixtures thereof, are within the scope of the embodiments. By way of non-limiting example, the mixture may be a racemate or the mixture may comprise unequal proportions of one particular stereoisomer over the other. Additionally, the compounds can be provided as a substantially pure stereoisomers, diastereomers and optical stereoisomers (such as epimers).

The compounds described herein can be asymmetric (e.g., having one or more stereocenters). All stereoisomers, such as enantiomers and diastereomers, are intended to be included within the scope of the embodiments unless otherwise indicated. Compounds that contain asymmetrically substituted carbon atoms can be isolated in optically active or racemic forms. Methods of preparation of optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are provided herein. Cis and trans geometric isomers of the compounds are also included within the present embodiments and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

In some embodiments, the composition comprises a compound, or a pharmaceutically acceptable salt, solvate or prodrug thereof, that is at least 90%, at least 95%, at least 98%, or at least 99%, or 100% enantiomeric pure, which means that the ratio of one enantiomer to the other in the composition is at least 90:1 at least 95:1, at least 98:1, or at least 99:1, or is completely in the form of one enantiomer over the other. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

Resolution of racemic mixtures of compounds can be carried out by any of numerous methods known in the art, including, for example, chiral HPLC, fractional recrystallization using a chiral resolving acid which is an optically active, salt-forming organic acid. Suitable resolving agents for fractional recrystallization methods include, but are not limited to, optically active acids, such as the D and L forms of tartaric acid, diacetyltartaric acid, dibenzoyltartaric acid, mandelic acid, malic acid, lactic acid, and the various optically active camphorsulfonic acids such as β-camphorsulfonic acid. Other resolving agents suitable for fractional crystallization methods include, but are not limited to, stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Resolution of racemic mixtures can also be carried out by elution on a column packed with an optically active resolving agent (e.g., dinitrobenzoylphenylglycine). Suitable elution solvent compositions can be determined by one skilled in the art.

Compounds may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Glycine transporter inhibitors, such as GlyT1 inhibitors, including their pharmaceutically acceptable salts (e.g., the GlyT1 inhibitors as disclosed herein) can also exist as hydrates and solvates, as well as anhydrous and non-solvated forms. A “hydrate” is a compound that exists in a composition with water molecules. The composition can include water in stoichiometric quantities, such as a monohydrate or a dihydrate, or can include water in random amounts. A “solvate” is a similar composition except that a solvent other that water, such as with methanol, ethanol, dimethylformamide, diethyl ether and the like replaces the water. For example, methanol or ethanol can form an “alcoholate,″” which can again be stoichiometic or non-stoichiometric. Mixtures of such solvates or hydrates can also be prepared. The source of such solvate or hydrate can be from the solvent of crystallization, inherent in the solvent of preparation or crystallization, or adventitious to such solvent.

The compounds of the application, including their pharmaceutically acceptable salts and prodrugs, can exist as various polymorphs, pseudo-polymorphs, or in amorphous state. The term “polymorph”, as used herein, refers to different crystalline forms of the same compound and other solid state molecular forms including pseudo-polymorphs, such as hydrates, solvates, or salts of the same compound. Different crystalline polymorphs have different crystal structures due to a different packing of molecules in the lattice, as a result of changes in temperature, pressure, or variations in the crystallization process. Polymorphs differ from each other in their physical properties, such as x-ray diffraction characteristics, stability, melting points, solubility, or rates of dissolution in certain solvents. Thus crystalline polymorphic forms are important aspects in the development of suitable dosage forms in pharmaceutical industry.

Compounds can also include all isotopes of atoms occurring in the intermediates or final compounds. Isotopes include those atoms having the same atomic number but different mass numbers. For example, isotopes of hydrogen include tritium and deuterium.

In some embodiments, the compounds, or salts thereof, are substantially isolated. Partial separation can include, for example, a composition enriched in the compound. Substantial separation can include compositions containing at least about 50%, at least about 60%, at least about 70%, at least about 80%, at least about 90%, at least about 95%, at least about 97%, or at least about 99% by weight of the compound, or salt thereof. Methods for isolating compounds and their salts are routine in the art.

Although the disclosed compounds are suitable, other functional groups can be incorporated into the compound with an expectation of similar results. In particular, thioamides and thioesters are anticipated to have very similar properties. The distance between aromatic rings can impact the geometrical pattern of the compound and this distance can be altered by incorporating aliphatic chains of varying length, which can be optionally substituted or can comprise an amino acid, a dicarboxylic acid or a diamine. The distance between and the relative orientation of monomers within the compounds can also be altered by replacing the amide bond with a surrogate having additional atoms. Thus, replacing a carbonyl group with a dicarbonyl alters the distance between the monomers and the propensity of dicarbonyl unit to adopt an anti-arrangement of the two carbonyl moiety and alter the periodicity of the compound. Pyromellitic anhydride represents still another alternative to simple amide linkages which can alter the conformation and physical properties of the compound. Modern methods of solid phase organic chemistry (E. Atherton and R. C. Sheppard, Solid Phase Peptide Synthesis A Practical Approach IRL Press Oxford 1989) now allow the synthesis of homodisperse compounds with molecular weights approaching 5,000 Daltons. Other substitution patterns are equally effective.

The compounds also include derivatives referred to as prodrugs.

Compounds containing an amine function can also form N-oxides. A reference herein to a compound that contains an amine function also includes the N-oxide. Where a compound contains several amine functions, one or more than one nitrogen atom can be oxidized to form an N-oxide. Examples of N-oxides include N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g., a peroxycarboxylic acid) (see, Advanced Organic Chemistry, by Jerry March, 4th Edition, Wiley Interscience).

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Embodiments of various compounds and salts thereof are provided. Where a variable is not specifically recited, the variable can be any option described herein, except as otherwise noted or dictated by context.

In some embodiments, the compound is as described in the appended exemplary, non-limiting claims, or a pharmaceutically acceptable salt, solvate or prodrug thereof.

In some embodiments of the methods and uses disclosed herein, the GlyT1 inhibitor is a compound of Formula I,

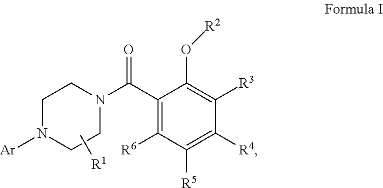

wherein:
Ar is unsubstituted or substituted aryl or 6-membered heteroaryl containing one, two or three nitrogen atoms, wherein the substituted aryl and the substituted heteroaryl groups are substituted by one or more substituents selected from the group consisting of hydroxy, halogen, NO₂, CN, (C₁-C₆)-alkyl, (C₁-C₆)-alkyl substituted by halogen, (C₁-C₆)-alkyl substituted by hydroxy, (CH₂)n-(C₁-C₆)-alkoxy, (C₁-C₆)-alkoxy substituted by halogen, NR⁷R⁸, C(O)R⁹, SO₂R¹⁰, and —C(CH₃)═NOR⁷, or are substituted by a 5-membered aromatic heterocycle containing 1-4 heteroatoms selected from N and O, which is optionally substituted by (C₁-C₆)-alkyl;
R¹ is hydrogen or (C₁-C₆)-alkyl;
R² is hydrogen, (C₁-C₆)-alkyl, (C₂-C₆)-alkenyl, (C₁-C₆)-alkyl substituted by halogen, (C₁-C₆)-alkyl substituted by hydroxy, (CH₂)n-(C₃-C₇)-cycloalkyl optionally substituted by (C₁-C₆)-alkoxy or by halogen, CH(CH₃)—(C₃-C₇)-cycloalkyl, (CH₂)ₙ₊₁—C(O)—R⁹, (CH₂)ₙ₊₁—CN, bicyclo[2.2.1]heptyl, (CH₂)ₙ₊₁—O—(C₁-C₆)-alkyl, (CH₂)n-heterocycloalkyl, (CH₂)ₙ-aryl or (CH₂)ₙ-5 or 6-membered heteroaryl containing one, two or three heteroatoms selected from the group consisting of oxygen, sulphur or nitrogen wherein aryl, heterocycloalkyl and heteroaryl are unsubstituted or substituted by one or more substituents selected from the group consisting of hydroxy, halogen, (C₁-C₆)-alkyl and (C₁-C₆)-alkoxy;
R³, R⁴ and R⁶ are each independently hydrogen, hydroxy, halogen, (C₁-C₆)-alkyl, (C₁-C₆)-alkoxy or O—(C₃-C₆)-cycloalkyl;
R⁵ is NO₂, CN, C(O)R⁹ or SO₂R¹⁰;
R⁷ and R⁸ are each independently hydrogen or (C₁-C₆)-alkyl;
R⁹ is hydrogen, (C₁-C₆)-alkyl, (C₁-C₆)-alkoxy or NR⁷R⁸;
R¹⁰ is (C₁-C₆)-alkyl optionally substituted by halogen, (CH₂)ₙ—(C₃-C₆)-cycloalkyl, (CH₂)ₙ—(C₃-C₆)-alkoxy, (CH₂)ₙ-heterocycloalkyl or NR⁷R⁸;
n is 0, 1, or 2;
or a pharmaceutically acceptable salt thereof, or a prodrug of the compound or its pharmaceutically acceptable salt.

In some embodiments of the methods and uses disclosed herein, the GlyT1 inhibitor is a compound having a formula of

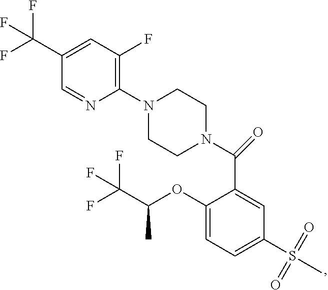 [SMILES: C[C@H](Oc1ccc(S(C)(=O)=O)cc1C(=O)N1CCN(c2ncc(C(F)(F)F)cc2F)CC1)C(F)(F)F]

bitopertin, or a pharmaceutically acceptable salt thereof, or a prodrug of the compound or its pharmaceutically acceptable salt.

In some embodiments of the methods and uses disclosed herein, the GlyT1 inhibitor is a compound of Formula II,

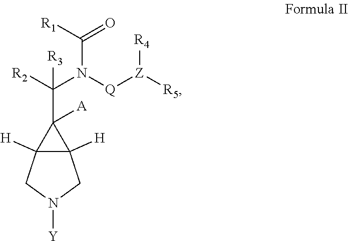

wherein:
R₁ represents a heteroaryl selected from the group consisting of: imidazolyl, thiazolyl, pyridyl, oxazolyl, pyrazolyl, triazolyl, oxadiazolyl, quinolinyl, isoxazolyl, pyrroloimidazoyl, and thiadiazole, wherein said heteroaryl is optionally substituted by one or more substituents selected from —OH, —NR₇R₈, halogen, (C₁-C₈)alkyl, (C₃-C₁₀)cycloalkyl, (C₁-C₈)alkoxy, (C₁-C₁₂)alkoxyalkyl, (C₁-C₈)hydroxyalkyl, (C₆-C₁₄)aryl and benzyl;
R₂, R₃ and A independently represent H or (C₁-C₈)alkoxy, wherein said alkyl is optionally substituted by one or more —OH, (C₁-C₈)alkoxy, —NR₇R₈ or halogen;
Q represents —(CH₂)ₙ—, where n=1, 2, 3 or 4 or —(CH₂)ₘ—O—, where m=2, 3 or 4;
Z represents (C₆-C₁₄)aryl, (C₁-C₈)alkyl or (C₃-C₈)cycloalkyl;
R₄ and R₅ each independently represent H, halogen, (C₁-C₈)alkyl, (C₆-C₁₄)aryl, (C₆-C₁₄)aryloxy, (C₁-C₈)alkoxy, (3-10 membered)heterocycloalkyl or (C₃-C₈)cycloalkoxy; wherein R₄ and R₅ are optionally substituted by one or more —OH, (C₁-C₈)alkoxy, —NR₇R₈ or halogen;
Y represents —R₆, —(CH₂)o-R₆, —C(R₆)₃ or —CH(R₆)₂, wherein 0=1, 2 or 3;
R₆ represents H, (C₆-C₁₄)aryl, (C₁₋₁₀)alkyl, (C₃-C₁₀)cycloalkyl, (C₅-C₁₈)bicycloalkyl, (C₅-C₁₈)tricycloalkyl, (3-10 membered)heterocycloalkyl, (5-10 membered)heteroaryl, —C(═O)NR₇R₈, or —C(═O)OR₇, wherein said R₆ groups can optionally be substituted with one or more X groups;
wherein X=—OH, (C₁-C₈)alkoxy, —NR₁₁R₁₂, —SO₂R₁₀, —C(═O)R₁₀, halogen, cyano, (C₁-C₈)alkyl, (C₁-C₁₀)alkoxyalkyl, (5-10 membered)heteroaryl, (C₆-C₁₄)aryl, (C₆-C₁₄)aryloxy, benzyl, or (C₁-C₈)hydroxyalkyl;
wherein R₇ and R₈ independently represent H, (C₁-C₈)alkyl, (C₃-C₈)cycloalkyl, (5-10 membered)heterocycloalkyl, (C₁-C₈)hydroxyalkyl, (5-10 membered)heteroaryl or (C₁-C₁₀)alkoxyalkyl; wherein R₇ and R₈ may optionally be substituted by one or more X groups;
or R₇ and R₈ together with the nitrogen in which they may be attached may form a (3-membered)heterocycloalkyl group optionally substituted by one or more X groups;
wherein R₁₀ represents (C₁-C₈)alkyl, (C₃-C₈)cycloalkyl, (3-10 membered)heterocycloalkyl, (C₁-C₈)hydroxyalkyl, (5-10 membered)heteroaryl or (C₁-C₁₀)alkoxyalkyl;
wherein R₁₁ and R₁₂ independently represent H, (C₁-C₈)alkyl, (C₃-C₈)cycloalkyl, (5-membered)heterocycloalkyl, (C₁-C₈)hydroxyalkyl, (5-10 membered)heteroaryl or (C₁-C₁₀)alkoxyalkyl; or a pharmaceutically acceptable salt thereof, or a prodrug of the compound or its pharmaceutically acceptable salt.

In some embodiments of the methods and uses disclosed herein, the GlyT1 inhibitor is a compound having a formula of

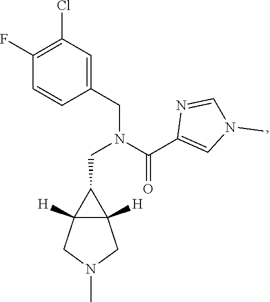 [SMILES: CN1C[C@@H]2[C@H](C1)[C@@H]2CN(Cc1ccc(F)c(Cl)c1)C(=O)c1cn(C)cn1]

or a pharmaceutically acceptable salt thereof, or a prodrug of the compound or its pharmaceutically acceptable salt.

In some embodiments of the methods and uses disclosed herein, the GlyT1 inhibitor is a compound having a formula of

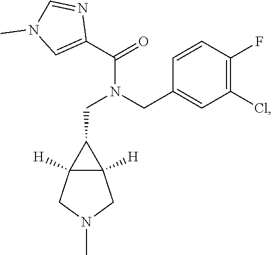 [SMILES: CN1C[C@@H]2[C@H](C1)[C@H]2CN(Cc1ccc(F)c(Cl)c1)C(=O)c1cn(C)cn1]

PF-3463275, or a pharmaceutically acceptable salt thereof, or a prodrug of the compound or its pharmaceutically acceptable salt.

In some embodiments of the methods and uses disclosed herein, the GlyT1 inhibitor is a compound of Formula III,

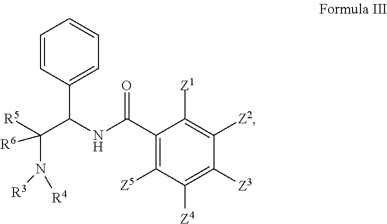

wherein:
Z¹ is selected from the group consisting of C₁₋₄alkyl, C₃₋₆Cycloalkyl, C₁₋₄alkoxy, C₁₋₄alkylthio, haloC₁₋₄alkyl, phenyl, haloC₁₋₄alkoxy, halophenyl, C₁₋₄alkylsulfoxy, C₁₋₄alkylsulfonyl, bromo and chloro;
Z² is selected from the group consisting of hydrogen, halogen, cyano, C₁₋₄alkyl, phenyl, haloC₁₋₄alkyl, haloC₁₋₄alkoxy, halophenyl, C₁₋₄alkoxyC₁₋₄alkyl and C₃₋₆cycloalkyl;
Z³ is selected from the group consisting of hydrogen, halogen, C₁₋₄alkyl, C₁₋₄alkoxy, C₁₋₄alkylthio, haloC₁₋₄alkyl, haloC₁₋₄alkoxy, and C₃₋₆cycloalkyl;
Z⁴ is selected from the group consisting of hydrogen, halogen, C₁₋₃alkyl, haloC₁₋₄alkyl, C₁₋₄alkoxy, C₁₋₄alkylthio, phenyl, haloC₁₋₄alkoxy, halophenyl, C₁₋₄alkoxyC₁₋₄alkyl and C₃₋₆cycloalkyl;
Z⁵ is selected from the group consisting of hydrogen, fluoro, chloro, bromo, iodo, hydroxy, C₁₋₄alkyl, C₁₋₄alkoxy, C₁₋₄alkylthio, phenyl, haloC₁₋₄alkyl, haloC₁₋₄alkoxy, halophenyl, C₁₋₄alkoxyC₁₋₄alkyl and C₃₋₆cycloalkyl;
whereby if more than one of Z¹ to Z⁵ is methoxy, then only Z¹ and Z⁵ are methoxy R³ and R⁴ are independently selected from hydrogen and C₁₋₄alkyl, optionally substituted with one or more groups Y; or R³ and R4 together with the nitrogen atom to which they are attached form a saturated or partially unsaturated A-, 5- 6- or 7-membered carbocyclic ring optionally substituted with a group Y′;
Y is selected from the group consisting of C₁₋₄alkoxy, hydroxy, haloC₁₋₄alkoxy and C₃₋₅cycloalkyl;
Y′ is selected from the group consisting of C₁₋₄alkyl, C₁₋₄alkoxy, halogen, hydroxy, haloC₁₋₄alkoxy, C₃₋₅cycloalkyl and C₅₋₁₀aryl or Y′ forms a —CH2- or —CH2-CH2- bridge between two atoms on the A-, 5-, 6- or 7-membered carbocyclic ring;
R⁵ and R⁶ are independently C₁₋₄alkyl, optionally substituted with one or more groups X; or R⁵ and R⁶ together with the carbon atom to which they are attached form a saturated 5- or 6-membered ring carbocyclic optionally substituted with one or more groups X′, in the case of R⁵ and R6 together with the carbon atom to which they are attached forming a 5-membered saturated carbocyclic ring, that ring may optionally further comprising an additional heteroatom group selected from O, N and S(O)m; where m=0, 1 or 2.
X is selected from the group consisting of halogen, hydroxy, C₁₋₄alkoxy, haloC₁₋₄alkyl, haloC₁₋₄alkoxy and C₅₋₁₀aryl; and
X is selected from the group consisting of halogen, hydroxy, C₁₋₄alkyl, C₁₋₄alkoxy, haloC₁₋₄alkyl, haloC₁₋₄alkoxy and C₅₋₁₀aryl;
whereby R³, R⁴, R⁵ and R⁶ are not all simultaneously unsubstituted methyl;
with the provisos that when simultaneously Z¹ is propyloxy, Z³ is chloro, Z²=Z⁴=Z⁵═H, and R⁵ and R⁶ are both methyl, then R³ and R⁴ together with the nitrogen atom to which they are attached do not form a 2-methylpyrrolidine group; when simultaneously Z¹ is methyl, Z³ is methoxy, Z²=Z4=Z5=H, and R⁵ and R⁶ are both methyl, then R³ and R⁴ together with the nitrogen atom to which they are attached do not form a pyrrolidine group, or a pharmaceutically acceptable salt thereof, or a prodrug of the compound or its pharmaceutically acceptable salt.

In some embodiments of the methods and uses disclosed herein, the GlyT1 inhibitor is a compound having a formula of

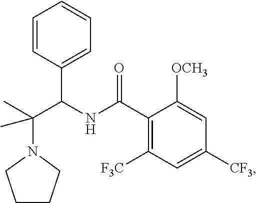 [SMILES: COc1cc(C(F)(F)F)cc(C(F)(F)F)c1C(=O)NC(c1ccccc1)C(C)(C)N1CCCC1]

or a pharmaceutically acceptable salt thereof, or a prodrug of the compound or its pharmaceutically acceptable salt.

In some embodiments of the methods and uses disclosed herein, the GlyT1 inhibitor is a compound of Formula IV,

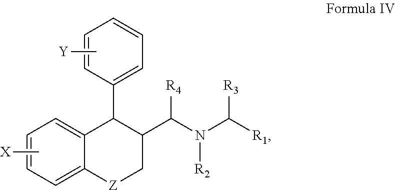

wherein:
Z is (CH₂)ₙ, O, S, SO, SO₂ or N—R₅;
n is 0, 1 or 2;
X represents 1-3 substituents independently selected from hydrogen, halogen,
(C₁₋₆)alkyloxy, (C₃₋₆)cycloalkyloxy, (C₆₋₁₂)aryloxy, (C₆₋₁₂)aryl, thienyl, SR6, SOR6,
SO₂R₆, NR₆R₆, NHR₆, NH₂, NHCOR₆, NSO₂R₆, CN, COOR₆ and (C₁₋₄)alkyl, optionally substituted with halogen, (C₆₋₁₂)aryl, (C₁₋₆)alkyloxy or (C₆₋₁₂)aryloxy; or 2 substituents at adjacent positions together represent a fused (C₅₋₆)aryl group, a fused (C₅₋₆)cycloalkyl ring or O—(CH₂)ₘ—O; m is 1 or 2;
Y represents 1-3 substituents independently selected from hydrogen, halogen, (C₁₋₄)alkyloxy, SR₆, NR₆R₆ and (C₁₋₄)alkyl, optionally substituted with halogen;
R₁ is COOR₇ or CONR₈R₉;
R₂ and R₆ are (C₁₋₄)alkyl;
R₃, R₄ are R₅ are independently hydrogen or (C₁₋₄)alkyl;
R₇, R₈ and R₉ are independently hydrogen, (C₁₋₄)alkyl, (C₆₋₁₂)aryl or arylalkyl, or a pharmaceutically acceptable salt thereof, or a prodrug of the compound or its pharmaceutically acceptable salt.

In some embodiments of the methods and uses disclosed herein, the GlyT1 inhibitor is a compound having a formula of

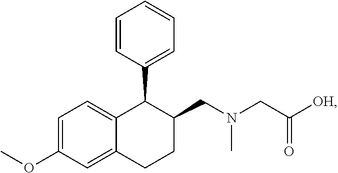 [SMILES: COc1ccc2c(c1)CC[C@H](CN(C)CC(=O)O)[C@@H]2c1ccccc1]

ORG-25935, or a pharmaceutically acceptable salt thereof, or a prodrug of the compound or its pharmaceutically acceptable salt.

In some embodiments of the methods and uses disclosed herein, the GlyT1 inhibitor is a compound of Formula V,

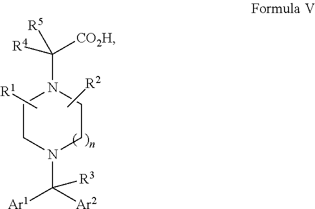

wherein:
n is an integer from 1 to 3;
R¹ and R² are independently selected from hydrogen, alkyl, haloalkyl, alkoxy, haloalkoxy, aryl, heteroaryl, cycloalkyl, or heterocyclyl wherein the aforementioned rings are optionally substituted with R^{a}, R^{b}, or R^{c} independently selected from alkyl, halo, haloalkyl, alkoxy, haloalkoxy, hydroxy, cyano, monosubstituted amino, or disubstituted amino; or
R¹ and R², when attached to the same carbon atom, can combine to form cycloalkyl or monocyclic saturated heterocyclyl to give a spiro ring wherein the cycloalkyl or monocyclic saturated heterocyclyl can be optionally substituted with R^{d}, R^{e}, or R^{f} independently selected from alkyl, alkoxy, fluoro, fluoroalkyl, fluoroalkoxy, hydroxy, monosubstituted amino, or disubstituted amino; or
R¹ and R², when attached to carbon atoms 2 and 5 or 3 and 6 positions of the piperazine ring, can combine to form —C₁-C₃— alkylene chain wherein one of the carbon atoms in the alkylene chain is optionally replaced by a —NR—, —O—, —S(O)n- (where R is hydrogen or alkyl and n is 0-2) and further wherein one or two hydrogen atoms in the alkylene chain can be optionally substituted with one or two alkyl;
R³, R⁴ and R⁵ are independently hydrogen, alkyl, fluoro, or fluoroalkyl; and Ar¹ and Ar² are independently aryl, heteroaryl, cycloalkyl, or heterocyclyl where each of the aforementioned ring is optionally substituted with R^{g}, R^{h} or Rⁱ where R^{g} is alkyl, —C═C—R⁶ (where R⁶ is aryl or heteroaryl), halo, haloalkyl, haloalkoxy, alkylthio, cyano, alloxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{h} and Rⁱ are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, or heterocyclyl where the aromatic or alicyclic ring-in R^{g}, R^{h} and Rⁱ is optionally substituted with R^{j}, R^{k}, or R^{l} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino; or a pharmaceutically acceptable salt thereof provided that: the compound of Formula V is not 2-(4-benzhydrylpiperazin-1-yl)acetic acid, 2-(4-((4-chlorophenyl)(phenyl)methyl)piperazin-1-yl)acetic acid, 2-((2R,5S)-4-((R)-(4-(1H-tetrazol-5-yl)phenyl)(3-hydroxyphenyl)methyl)-2,5-dimethylpiperazin-1-yl)acetic acid, or 2-((2R,5S)-4-((R)-(4-cyanophenyl)(3-hydroxyphenyl)methyl)-2,5-dimethylpiperazin-1-yl)acetic acid, or a pharmaceutically acceptable salt thereof, or a prodrug of the compound or its pharmaceutically acceptable salt.

In some embodiments of the methods and uses disclosed herein, the GlyT1 inhibitor is a compound having a formula of

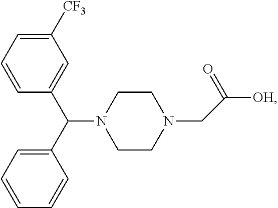 [SMILES: O=C(O)CN1CCN(C(c2ccccc2)c2cccc(C(F)(F)F)c2)CC1]

or a pharmaceutically acceptable salt thereof, or a prodrug of the compound or its pharmaceutically acceptable salt.

In some embodiments of the methods and uses disclosed herein, the GlyT1 inhibitor is a compound of Formula VI,

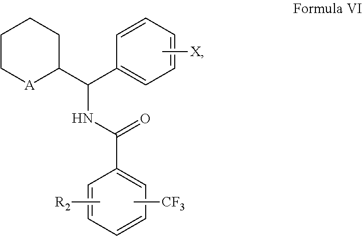

wherein:
A represents a group of general formula N—R₁, a group of general formula N+(O—)R₁ or a group of general formula N+(R′)R₁, and in which R₁ represents either a hydrogen atom, or a linear or branched (C₁-C₇)alkyl group optionally substituted with one or more fluorine atoms, or a (C₄-C₇)cycloalkyl group, or a (C₃-C₇)cycloalkyl(C₁-C₃)alkyl group, or a phenyl(C₁-C₃)alkyl group optionally substituted with one or two hydroxyl or methoxy groups, or a (C₂-C₄)alkenyl group, or a (C₂-C₄)alkynyl group,
R′ represents a linear or branched (C₁-C₇)alkyl group,
X represents a hydrogen atom or one or more substituents chosen from halogen atoms and trifluoromethyl, linear or branched (C₁-C₄)alkyl and (C₁-C₄)alkoxy groups,
R₂ represents either a hydrogen atom, or one or more substituents chosen from halogen atoms and trifluoromethyl, (C₁-C₄)alkyl or (C₁-C₄)alkoxy groups, or amino groups of general formula NR₃R₄ in which R₃ and R₄ each represent, independently of each other, a hydrogen atom or a (C₁-C₄)alkyl group, or form with the nitrogen atom carrying them a pyrrolidine, piperidine or morpholine ring, or a phenyl group optionally substituted with an atom or a group as defined for the symbol X above, or a pharmaceutically acceptable salt thereof, or a prodrug of the compound or its pharmaceutically acceptable salt.

In some embodiments of the methods and uses disclosed herein, the GlyT1 inhibitor is a compound having a formula of

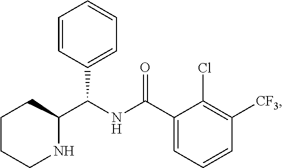 [SMILES: O=C(N[C@@H](c1ccccc1)[C@@H]1CCCCN1)c1cccc(C(F)(F)F)c1Cl]

SSR-504734, or a pharmaceutically acceptable salt thereof, or a prodrug of the compound or its pharmaceutically acceptable salt.

In some embodiments of the methods and uses disclosed herein, the GlyT1 inhibitor is a compound of Formula VII,

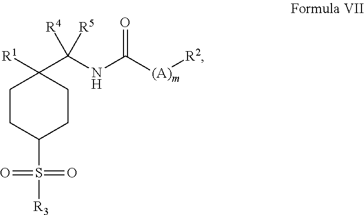

wherein:
R¹ is —(CH₂)ₙ—R^{1a}, wherein n is independently 0-6, and R^{1a} is selected from the group consisting of:
(1) C₁₋₆alkyl, which is unsubstituted or substituted with 1-6 halogen, hydroxy,
(2) phenyl substituted with R^{2a}, R^{2b} and R^{2c},
(3) C₃₋₆cycloalkyl, which is unsubstituted or substituted with C₁₋₆alkyl, 1-6 halogen, hydroxy or NR¹⁰R¹¹,
(4) —O—C₁₋₆alkyl, which is unsubstituted or substituted with 1-6 halogen, hydroxy or —NR¹⁰R¹¹,
(5) —CO2R⁹,
wherein R9 is independently selected from:
(a) hydrogen,
(b) —C₁₋₆alkyl, which is unsubstituted or substituted with 1-6 fluoro,
(c) benzyl, and
(d) phenyl,
(6) —NR¹⁰R¹¹,
wherein R¹⁰ and R¹¹ are independently selected from:
(a) hydrogen,
(b) —C₁₋₆alkyl, which is unsubstituted or substituted with hydroxy, 1-6 fluoro or NR¹²R¹³, where R¹² and R¹³ are independently selected from hydrogen and —C₁₋₆alkyl,
(c) C₃₋₆cycloalkyl, which is unsubstituted or substituted with hydroxy, 1-6 fluoro or —NR¹²R¹³,
(d) benzyl,
(e) phenyl, and
(7) —CONR¹⁰R¹¹;
R² is selected from the group consisting of:
(1) phenyl, which is substituted with R^{2a}, R^{2b} and R^{2c},
(2) C₁₋₈alkyl, which is unsubstituted or substituted with 1-6 halogen, hydroxy, —NR¹⁰R¹¹, phenyl or heterocycle, where the phenyl or heterocycle is substituted with R^{2a}, R^{2b} and R^{2c},
(3) C₃₋₆cycloalkyl, which is unsubstituted or substituted with 1-6 halogen, hydroxy or —NR¹⁰R¹¹, and
(4) —C₁₋₆alkyl-(C₃₋₆cycloalkyl), which is unsubstituted or substituted with 1-6 halogen, hydroxy or —NR¹⁰R¹¹;
R^{2a}, R^{2b} and R^{2c} are independently selected from the group consisting of:
(1) hydrogen,
(2) halogen,
(3) —C₁₋₆alkyl, which is unsubstituted or substituted with:
(a) 1-6 halogen,
(b) phenyl,
(c) C₃₋₆cycloalkyl, or
(d) —NR¹⁰R¹¹,
(4) —O—C₁₋₆alkyl, which is unsubstituted or substituted with 1-6 halogen,
(5) hydroxy,
(6) —SCF₃,
(7) —SCHF₂,
(8) —SCH₃,
(9) —CO₂R⁹,
(10) —CN,
(11) —SO₂R⁹,
(12) —SO₂—NR¹⁰R¹¹,
(13) —NR¹⁰R¹¹,
(14) —CONR¹⁰R¹¹, and
(15) —NO₂;
R³ is selected from the group consisting of:
(1) C₁₋₆alkyl, which is unsubstituted or substituted with 1-6 halogen, hydroxyl, or —NR¹⁰R¹¹,
(2) C₃₋₆cycloalkyl, which is unsubstituted or substituted with 1-6 halogen, hydroxyl or —NR¹⁰R¹¹,
R⁴ and R⁵ are independently selected from the group consisting of:
(1) hydrogen, and
(2) C₁₋₆alkyl, which is unsubstituted or substituted with halogen or hydroxyl, or R¹ and R⁵ taken together form a C₃₋₆cycloalkyl ring;
A is selected from the group consisting of:
(1) —O—, and
(2) —NR¹⁰—;
m is zero or one, whereby when m is zero R² is attached directly to the carbonyl;
and pharmaceutically acceptable salts thereof and individual enantiomers and diastereomers thereof, or a pharmaceutically acceptable salt thereof, or a prodrug of the compound or its pharmaceutically acceptable salt.

In some embodiments of the methods and uses disclosed herein, the GlyT1 inhibitor is a compound having a formula of

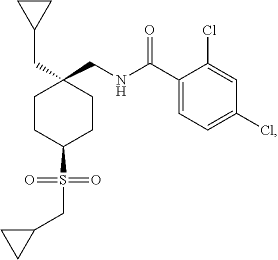 [SMILES: O=C(NC[C@]1(CC2CC2)CC[C@H](S(=O)(=O)CC2CC2)CC1)c1ccc(Cl)cc1Cl]

or a pharmaceutically acceptable salt thereof, or a prodrug of the compound or its pharmaceutically acceptable salt.

In some embodiments of the methods and uses disclosed herein, the GlyT1 inhibitor is a compound of Formula VIII,

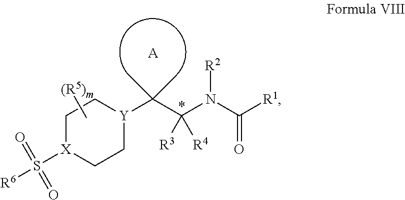

wherein:
R¹ is phenyl independently substituted from 1 to 5 times with halogen, C₁-C₃ alkyl, C₃-C₆cycloalkyl, OR⁹, or SR¹⁰, wherein C₁-C₃ alkyl and C₃-C₆ cycloalkyl are optionally substituted with 1 to 10 times with R⁷;
R² is H;
R³ and R4 are each individually H or CH₃;
R⁵ is selected from the group consisting of:
(1) hydrogen,
(2) C₁-C₆ alkyl which is optionally substituted from 1 to 11 times with R⁷,
(3) gem-dialkyl, and
(4) gem-dihalo; or
two R⁵ substituents on the same carbon, together with the carbon atom to which they are attached, may form a 3-, 4-, or 5-membered cycloalkyl optionally substituted from 1 to 10 times with R⁷; or
two R⁵ substituents on adjacent carbons of the ring to which they are attached, together may form a 3-, 4-, 5- or 6-membered cycloalkyl optionally substituted from 1 to 10 times with R⁷;
R⁶ is

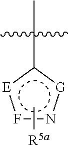

wherein E, F, and G are each independently nitrogen or carbon and R^{6a} is C₁-C₂ alkyl, which is optionally substituted 1 to 5 times with halogen or deuterium;
R⁷ is selected from the group consisting of:
(1) hydrogen,
(2) halogen,
(3) deuterium,
(4) gem-dialkyl,
(5) gem-dihalo,
(6) —OR⁹, —NR¹¹R¹², —NR¹¹C(O)ₚR¹⁰, —S(O)ₚR¹⁰, —CN, —NO2, —C(O)ₚR¹⁰, —C(O)NR¹¹R¹², or —NR¹¹C(S)R¹⁰, and
(7) oxo or thio;
R⁸ is selected from the group consisting of:
(1) hydrogen,
(2) halogen,
(3) C₁-C₆ alkyl, C₂-C₆ alkenyl, C₂-C₆ alkynyl, C₃-C₇ cycloalkyl, or C₄-C₇ cycloalkylalkyl,
wherein each of the C₁-C₆ alkyl, C₂-C₆ alkenyl, C₂-C₆ alkynyl, C₃-C₇ cycloalkyl, and C₄-C₇ cycloalkylalkyl is independently and optionally substituted from 1 to 11 times with R⁷, or
(4) —OR⁹, —NR¹¹R¹², —NR¹¹C(O)ₚR¹⁰, —S(O)ₚR¹⁰, —CN, —NO2, —C(O)ₚR¹⁰, —C(O)NR¹¹R¹², or —NR¹¹C(S)R¹⁰;
R⁹ is selected from the group consisting of hydrogen, C₁-C₄ alkyl, C₃-C₇ cycloalkyl, C₄-C₇ cycloalkylalkyl, C(O)NR¹¹R¹², and C(O)ₚR¹⁰, wherein each of C₁-C₄ alkyl, C₃-C₇ cycloalkyl, and C₄-C₇ cycloalkylalkyl is optionally substituted from 1 to 11 times with R⁷;
R¹⁰ is selected from the group consisting of hydrogen, C₁-C₄ alkyl, C₃-C₇ cycloalkyl C₄-C₇ cycloalkylalkyl, aryl, and heteroaryl, wherein each of C₁-C₄ alkyl, C₃-C₇ cycloalkyl, and C₄-C₇ cycloalkylalkyl is optionally substituted from 1 to 11 times with substituents as defined in R7 and aryl or heteroaryl is optionally substituted from 1 to 10 times with R⁸;
R¹¹ and R¹² are each independently selected from the group consisting hydrogen, C₁-C₄ alkyl, C₃-C₇ cycloalkyl, C₄-C₇ cycloalkylalkyl, aryl, and heteroaryl, wherein each of C₁-C₄ alkyl, C₃-C₇ cycloalkyl, and C₄-C₇ cycloalkylalkyl is optionally substituted from 1 to 11 times with substituents as defined in R⁷ and aryl or heteroaryl is optionally substituted from 1 to 10 times with R⁸, or R¹¹ and R¹² are taken together with the nitrogen to which they are attached to form a saturated or partially saturated monocyclic or fused bicyclic heterocycle optionally substituted from 1 to 11 times with R⁷;
A is

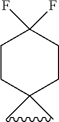

X is N;
Y is N;
p is 1, or 2; and
m is 0;
with the following provisos that: R⁶ cannot be (a) 1H-1,2,3-triazol-4-yl, or (b) 5-methylisoxazol-4-yl;
or an oxide thereof, a pharmaceutically acceptable salt of the compound or its oxide, or an individual enantiomer or diastereomer thereof.

In some embodiments of the methods and uses disclosed herein, the GlyT1 inhibitor is selected from any of the following:

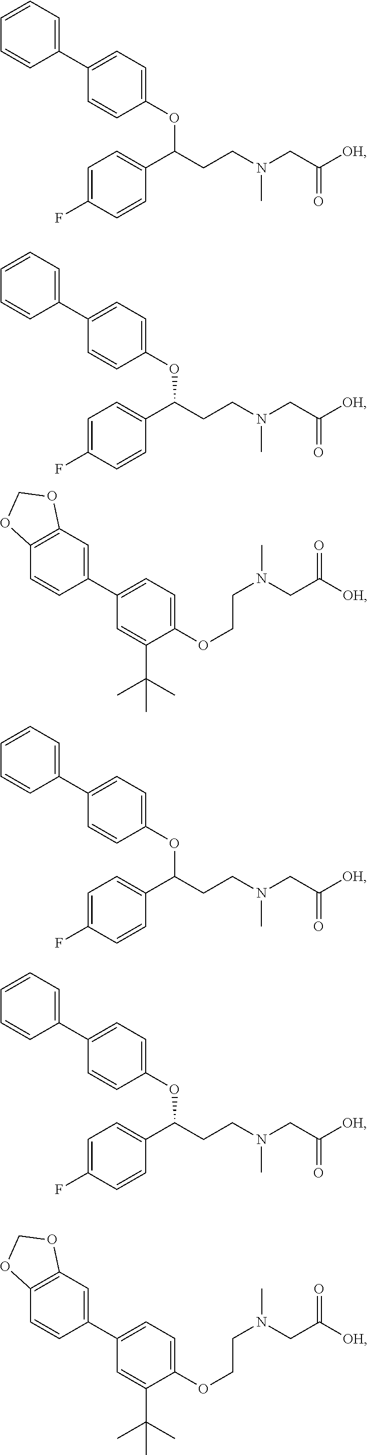

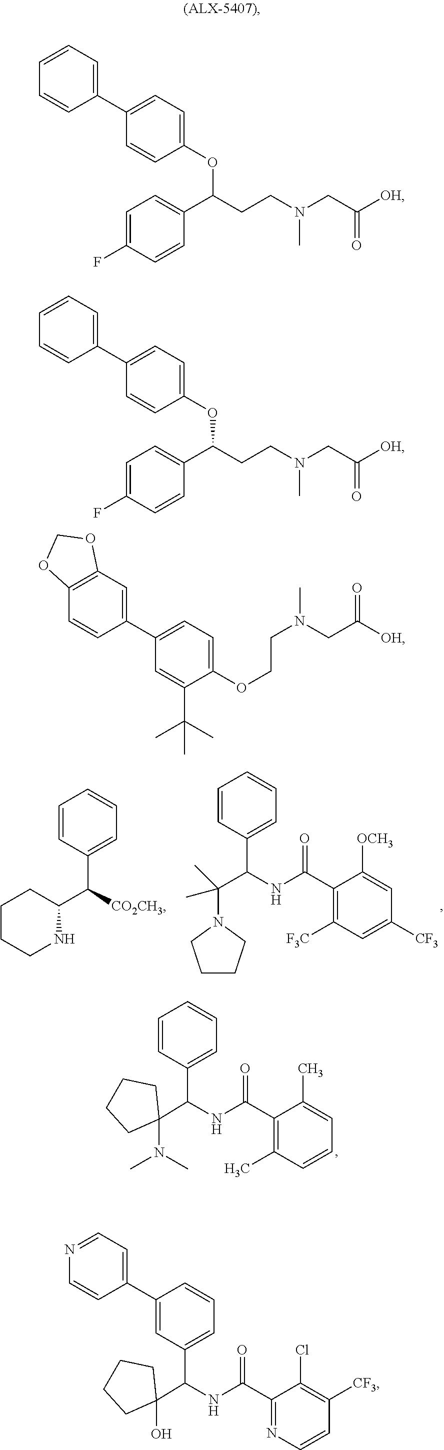

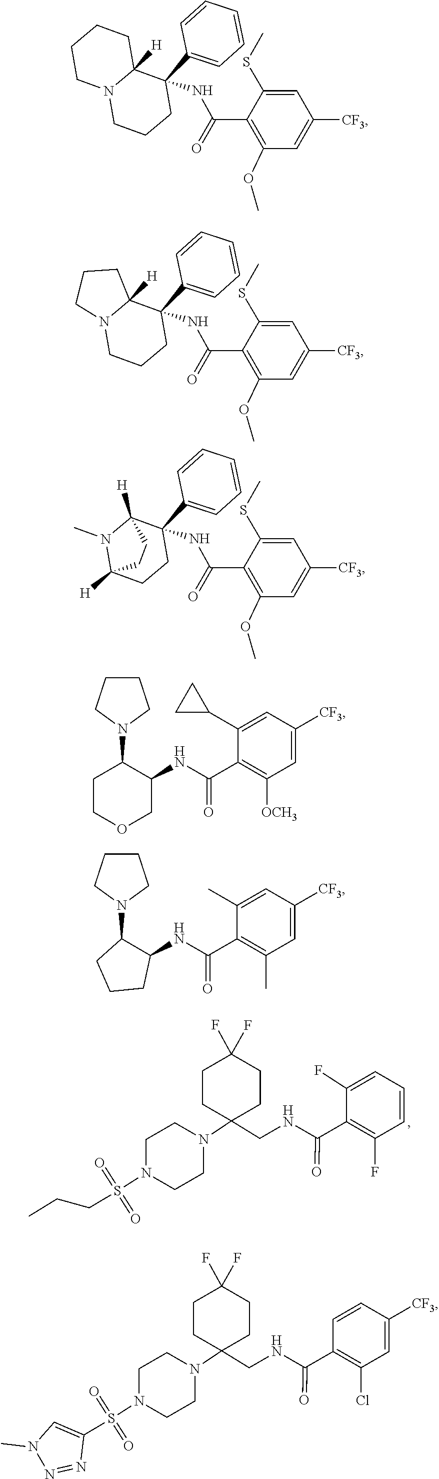

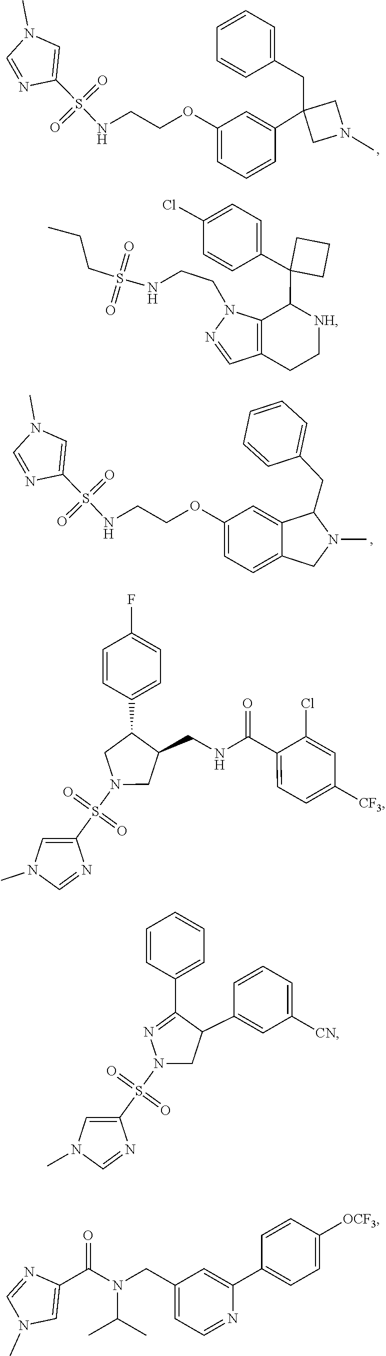

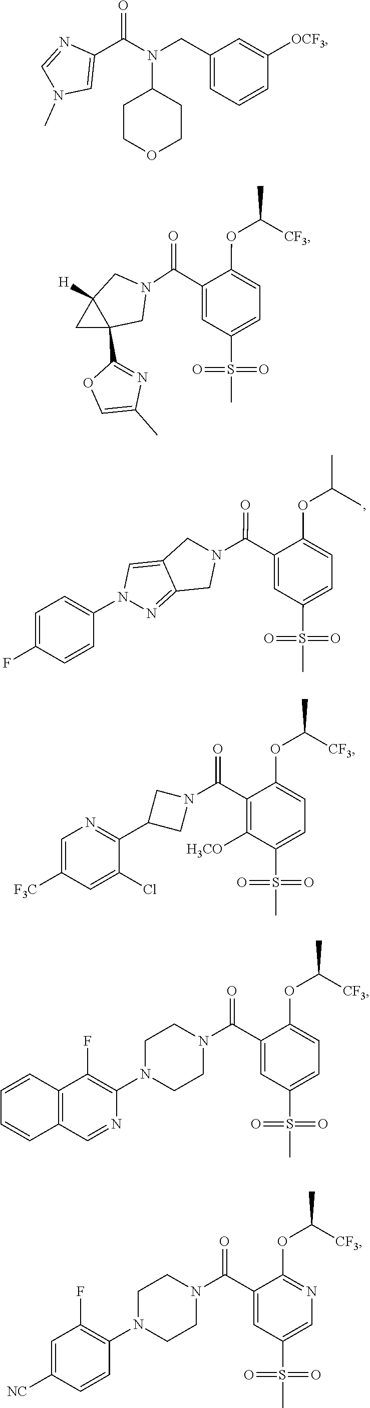

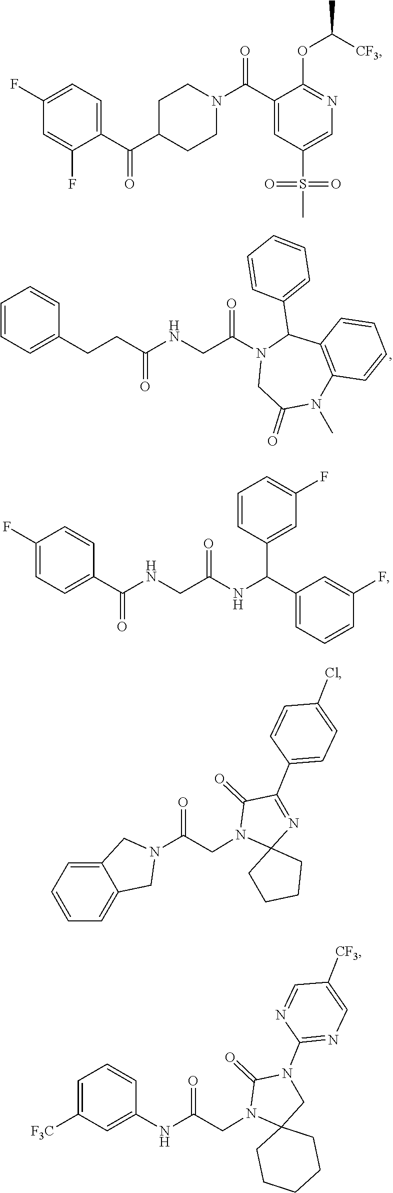

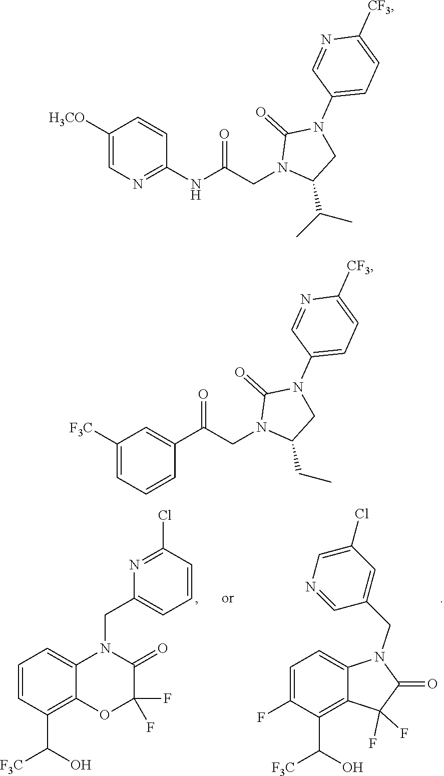

a pharmaceutically acceptable salt thereof, or a prodrug of the compound or its pharmaceutically acceptable salt.

In some embodiments of the methods and uses disclosed herein, the GlyT1 inhibitor is a compound having a formula of

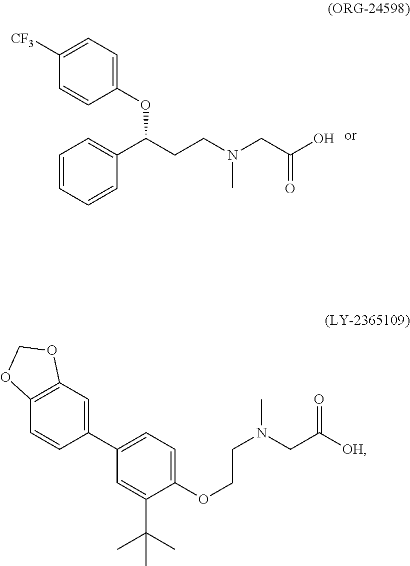 [SMILES: CN(CC[C@@H](Oc1ccc(C(F)(F)F)cc1)c1ccccc1)CC(=O)O ; CN(CCOc1ccc(-c2ccc3c(c2)OCO3)cc1C(C)(C)C)CC(=O)O]

or a pharmaceutically acceptable salt thereof, or a prodrug of the compound or its pharmaceutically acceptable salt.

In some embodiments of the methods and uses disclosed herein, the GlyT1 inhibitor is a compound of Formula IX,

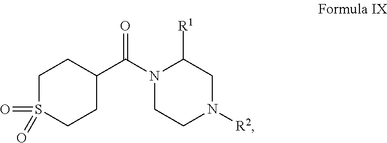

wherein:
R¹ represents phenyl or a 5 or 6 membered monocyclic heteroaryl having 1, 2, or 3 heteroatoms independently selected from O, N or S, wherein the phenyl or the heteroaryl is optionally substituted with one or more R³;
R² represents aryl, a 5 or 6 membered monocyclic heteroaryl or a 8 to 10 membered bicyclic heteroaryl, the mono- or bicyclic heteroaryl having 1, 2, or 3 heteroatoms independently selected from O, N or S, wherein the aryl or the heteroaryl is optionally substituted with one or more R⁴;
R³ is a halogen, a C₁₋₄-alkyl or a C₃₋₆-cycloalkyl, wherein the C₁₋₄-alkyl or the C₃₋₆-cycloalkyl is optionally substituted with one or more halogens; and
R⁴ is a halogen, —CN, C₁₋₄-alkyl. C₃₋₆-cycloalkyl, —C₁₋₃-alkyl —C₃₋₆-cycloalkyl or —O—C₁₋₆ alkyl, wherein the C₁₋₄-alkyl, C₃₋₆-cycloalkyl, C₁₋₃-alkyl C₃₋₆-cycloalkyl or the —O—C₁₋₆-alkyl is optionally substituted with one or more halogens;
or a pharmaceutically acceptable salt thereof, or a tautomer or stereoisomer of the compound or its pharmaceutically acceptable salt, or a mixture of any of the foregoing.

In certain embodiments, the compound of Formula IX can be represented by a compound of formula IX(a):

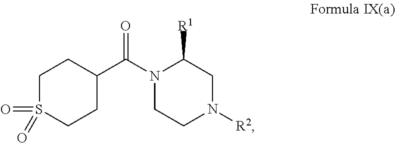

or a pharmaceutically acceptable salt thereof, or a tautomer the compound or its pharmaceutically acceptable salt, or a mixture of any of the foregoing.

In certain embodiments, the compound of Formula IX can be represented by a compound of formula IX(b):

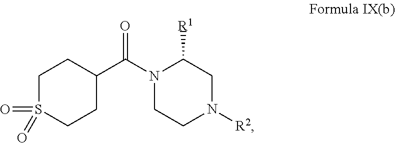

or a pharmaceutically acceptable salt thereof, or a tautomer the compound or its pharmaceutically acceptable salt, or a mixture of any of the foregoing.

In certain embodiments, the compound of formula IX is a compound selected from any of the following, a stereoisomer or stereoisomeric mixture thereof, or a pharmaceutically acceptable salt thereof:

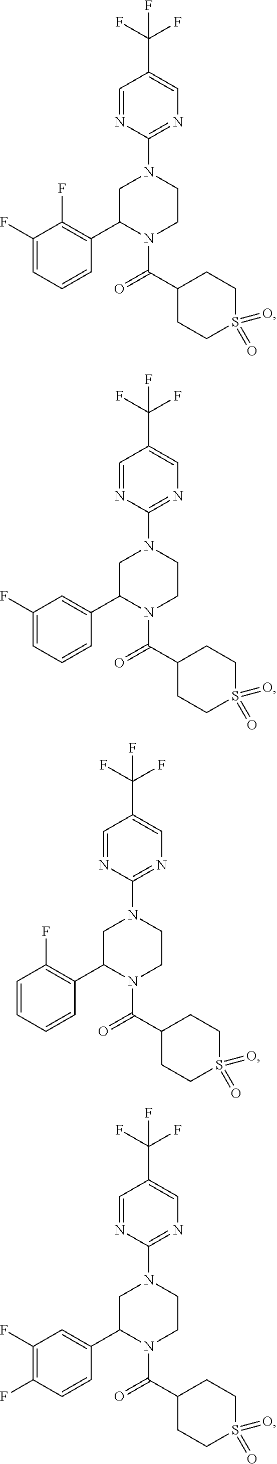 [SMILES: O=C(C1CCS(=O)(=O)CC1)N1CCN(c2ncc(C(F)(F)F)cn2)CC1c1cccc(F)c1F ; O=C(C1CCS(=O)(=O)CC1)N1CCN(c2ncc(C(F)(F)F)cn2)CC1c1cccc(F)c1 ; O=C(C1CCS(=O)(=O)CC1)N1CCN(c2ncc(C(F)(F)F)cn2)CC1c1ccccc1F ; O=C(C1CCS(=O)(=O)CC1)N1CCN(c2ncc(C(F)(F)F)cn2)CC1c1ccc(F)c(F)c1]

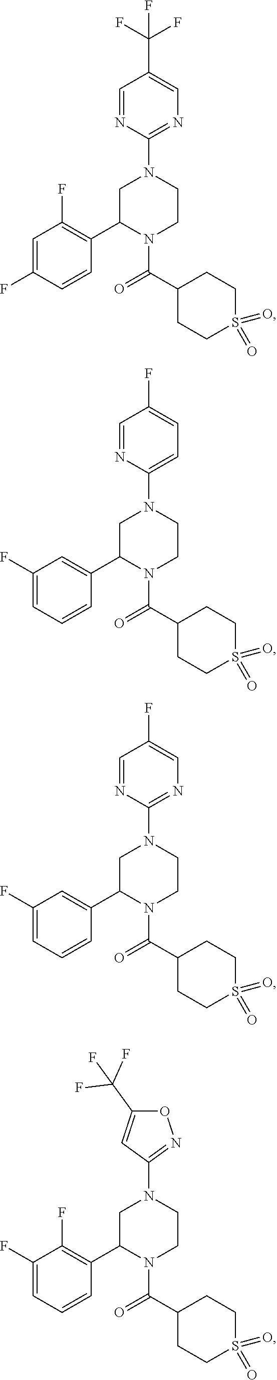 [SMILES: O=C(C1CCS(=O)(=O)CC1)N1CCN(c2ncc(C(F)(F)F)cn2)CC1c1ccc(F)cc1F ; O=C(C1CCS(=O)(=O)CC1)N1CCN(c2ccc(F)cn2)CC1c1cccc(F)c1 ; O=C(C1CCS(=O)(=O)CC1)N1CCN(c2ncc(F)cn2)CC1c1cccc(F)c1 ; O=C(C1CCS(=O)(=O)CC1)N1CCN(c2cc(C(F)(F)F)on2)CC1c1cccc(F)c1F]

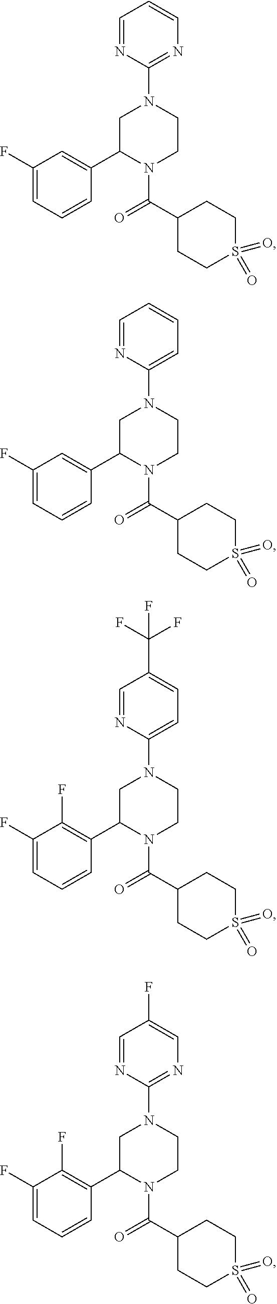 [SMILES: O=C(C1CCS(=O)(=O)CC1)N1CCN(c2ncccn2)CC1c1cccc(F)c1 ; O=C(C1CCS(=O)(=O)CC1)N1CCN(c2ccccn2)CC1c1cccc(F)c1 ; O=C(C1CCS(=O)(=O)CC1)N1CCN(c2ccc(C(F)(F)F)cn2)CC1c1cccc(F)c1F ; O=C(C1CCS(=O)(=O)CC1)N1CCN(c2ncc(F)cn2)CC1c1cccc(F)c1F]

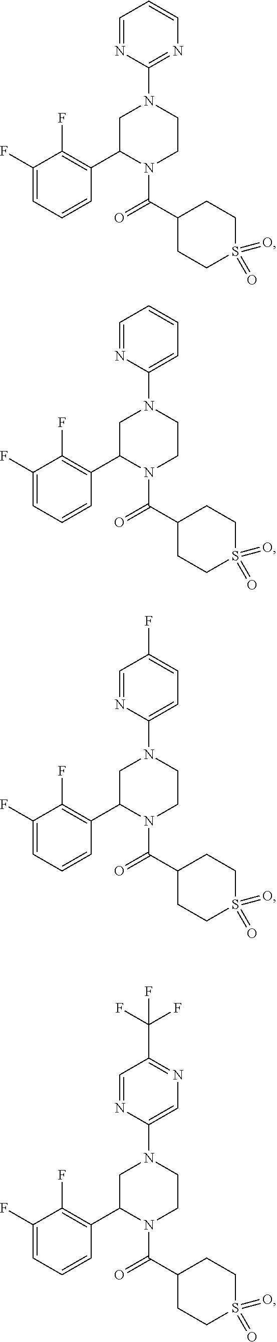 [SMILES: O=C(C1CCS(=O)(=O)CC1)N1CCN(c2ncccn2)CC1c1cccc(F)c1F ; O=C(C1CCS(=O)(=O)CC1)N1CCN(c2ccccn2)CC1c1cccc(F)c1F ; O=C(C1CCS(=O)(=O)CC1)N1CCN(c2ccc(F)cn2)CC1c1cccc(F)c1F ; O=C(C1CCS(=O)(=O)CC1)N1CCN(c2cnc(C(F)(F)F)cn2)CC1c1cccc(F)c1F]

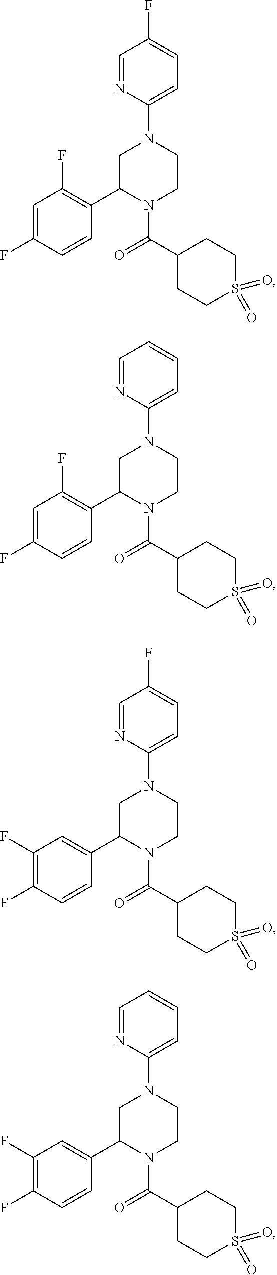 [SMILES: O=C(C1CCS(=O)(=O)CC1)N1CCN(c2ccc(F)cn2)CC1c1ccc(F)cc1F ; O=C(C1CCS(=O)(=O)CC1)N1CCN(c2ccccn2)CC1c1ccc(F)cc1F ; O=C(C1CCS(=O)(=O)CC1)N1CCN(c2ccc(F)cn2)CC1c1ccc(F)c(F)c1 ; O=C(C1CCS(=O)(=O)CC1)N1CCN(c2ccccn2)CC1c1ccc(F)c(F)c1]

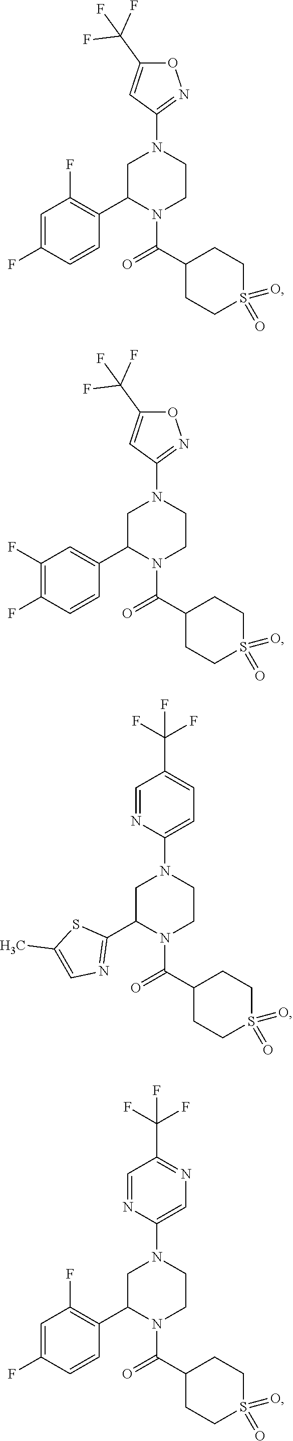 [SMILES: O=C(C1CCS(=O)(=O)CC1)N1CCN(c2cc(C(F)(F)F)on2)CC1c1ccc(F)cc1F ; O=C(C1CCS(=O)(=O)CC1)N1CCN(c2cc(C(F)(F)F)on2)CC1c1ccc(F)c(F)c1 ; Cc1cnc(C2CN(c3ccc(C(F)(F)F)cn3)CCN2C(=O)C2CCS(=O)(=O)CC2)s1 ; O=C(C1CCS(=O)(=O)CC1)N1CCN(c2cnc(C(F)(F)F)cn2)CC1c1ccc(F)cc1F]

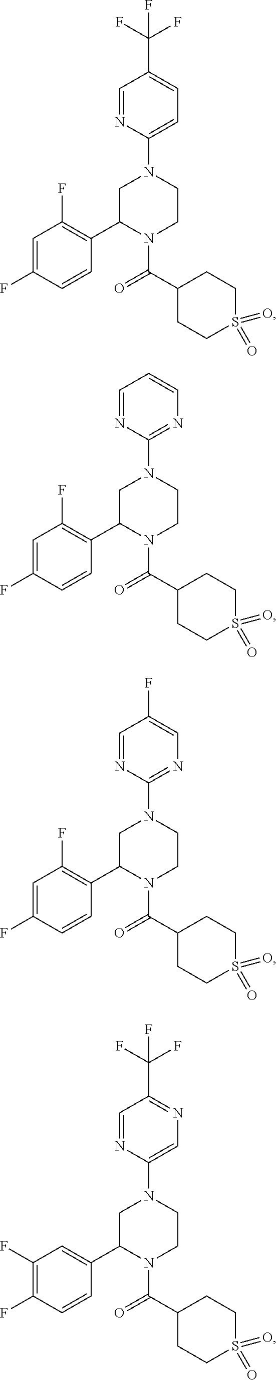 [SMILES: O=C(C1CCS(=O)(=O)CC1)N1CCN(c2ccc(C(F)(F)F)cn2)CC1c1ccc(F)cc1F ; O=C(C1CCS(=O)(=O)CC1)N1CCN(c2ncccn2)CC1c1ccc(F)cc1F ; O=C(C1CCS(=O)(=O)CC1)N1CCN(c2ncc(F)cn2)CC1c1ccc(F)cc1F ; O=C(C1CCS(=O)(=O)CC1)N1CCN(c2cnc(C(F)(F)F)cn2)CC1c1ccc(F)c(F)c1]

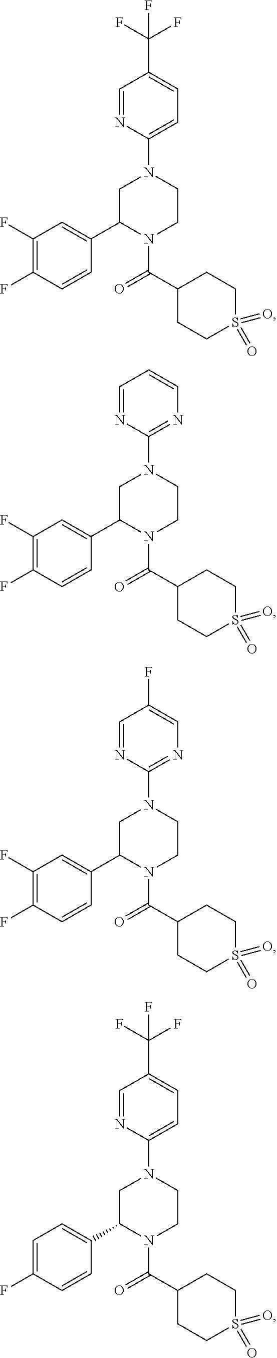 [SMILES: O=C(C1CCS(=O)(=O)CC1)N1CCN(c2ccc(C(F)(F)F)cn2)CC1c1ccc(F)c(F)c1 ; O=C(C1CCS(=O)(=O)CC1)N1CCN(c2ncccn2)CC1c1ccc(F)c(F)c1 ; O=C(C1CCS(=O)(=O)CC1)N1CCN(c2ncc(F)cn2)CC1c1ccc(F)c(F)c1 ; O=C(C1CCS(=O)(=O)CC1)N1CCN(c2ccc(C(F)(F)F)cn2)C[C@H]1c1ccc(F)cc1]

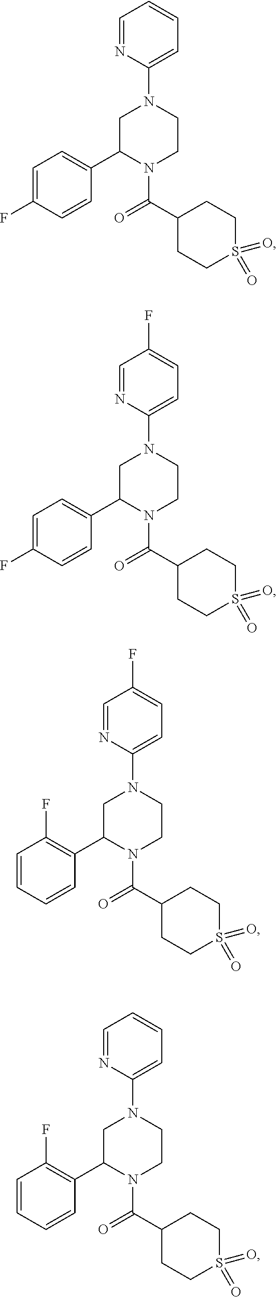 [SMILES: O=C(C1CCS(=O)(=O)CC1)N1CCN(c2ccccn2)CC1c1ccc(F)cc1 ; O=C(C1CCS(=O)(=O)CC1)N1CCN(c2ccc(F)cn2)CC1c1ccc(F)cc1 ; O=C(C1CCS(=O)(=O)CC1)N1CCN(c2ccc(F)cn2)CC1c1ccccc1F ; O=C(C1CCS(=O)(=O)CC1)N1CCN(c2ccccn2)CC1c1ccccc1F]

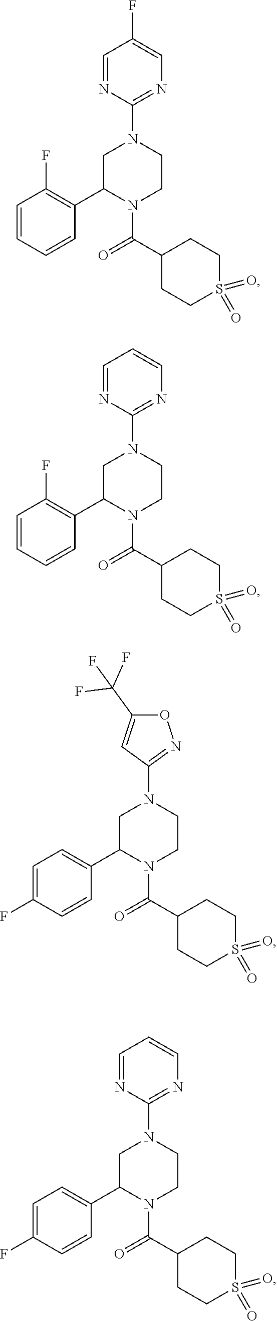 [SMILES: O=C(C1CCS(=O)(=O)CC1)N1CCN(c2ncc(F)cn2)CC1c1ccccc1F ; O=C(C1CCS(=O)(=O)CC1)N1CCN(c2ncccn2)CC1c1ccccc1F ; O=C(C1CCS(=O)(=O)CC1)N1CCN(c2cc(C(F)(F)F)on2)CC1c1ccc(F)cc1 ; O=C(C1CCS(=O)(=O)CC1)N1CCN(c2ncccn2)CC1c1ccc(F)cc1]

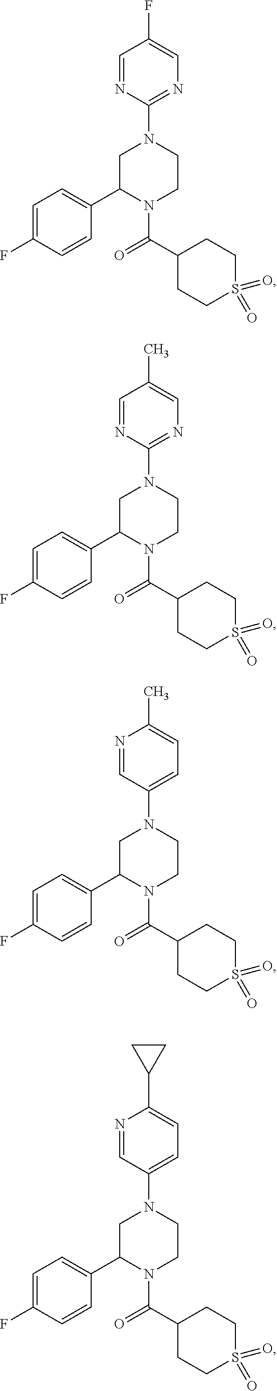 [SMILES: O=C(C1CCS(=O)(=O)CC1)N1CCN(c2ncc(F)cn2)CC1c1ccc(F)cc1 ; Cc1cnc(N2CCN(C(=O)C3CCS(=O)(=O)CC3)C(c3ccc(F)cc3)C2)nc1 ; Cc1ccc(N2CCN(C(=O)C3CCS(=O)(=O)CC3)C(c3ccc(F)cc3)C2)cn1 ; O=C(C1CCS(=O)(=O)CC1)N1CCN(c2ccc(C3CC3)nc2)CC1c1ccc(F)cc1]

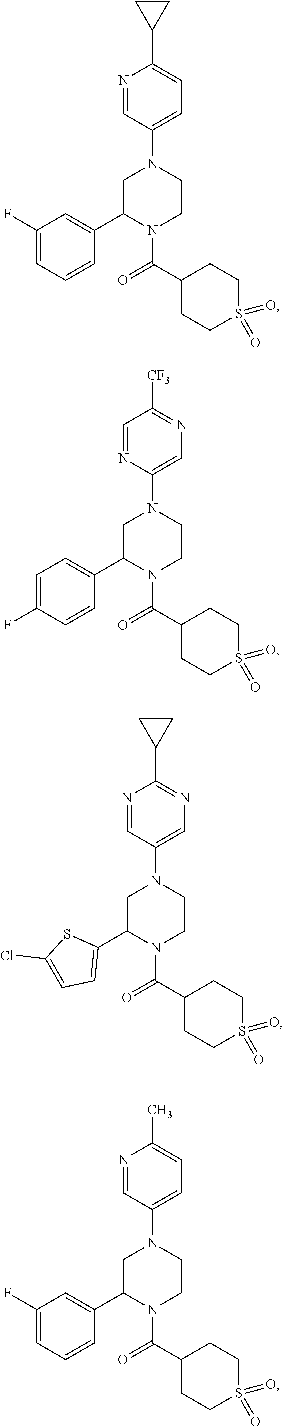 [SMILES: O=C(C1CCS(=O)(=O)CC1)N1CCN(c2ccc(C3CC3)nc2)CC1c1cccc(F)c1 ; O=C(C1CCS(=O)(=O)CC1)N1CCN(c2cnc(C(F)(F)F)cn2)CC1c1ccc(F)cc1 ; O=C(C1CCS(=O)(=O)CC1)N1CCN(c2cnc(C3CC3)nc2)CC1c1ccc(Cl)s1 ; Cc1ccc(N2CCN(C(=O)C3CCS(=O)(=O)CC3)C(c3cccc(F)c3)C2)cn1]

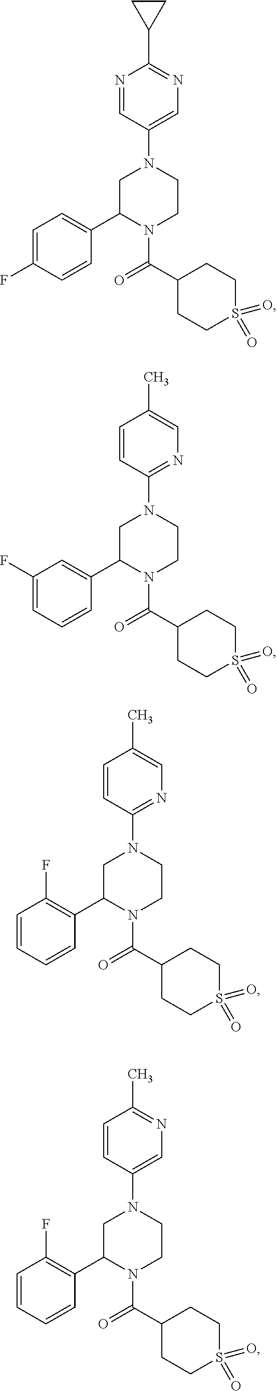 [SMILES: O=C(C1CCS(=O)(=O)CC1)N1CCN(c2cnc(C3CC3)nc2)CC1c1ccc(F)cc1 ; Cc1ccc(N2CCN(C(=O)C3CCS(=O)(=O)CC3)C(c3cccc(F)c3)C2)nc1 ; Cc1ccc(N2CCN(C(=O)C3CCS(=O)(=O)CC3)C(c3ccccc3F)C2)nc1 ; Cc1ccc(N2CCN(C(=O)C3CCS(=O)(=O)CC3)C(c3ccccc3F)C2)cn1]

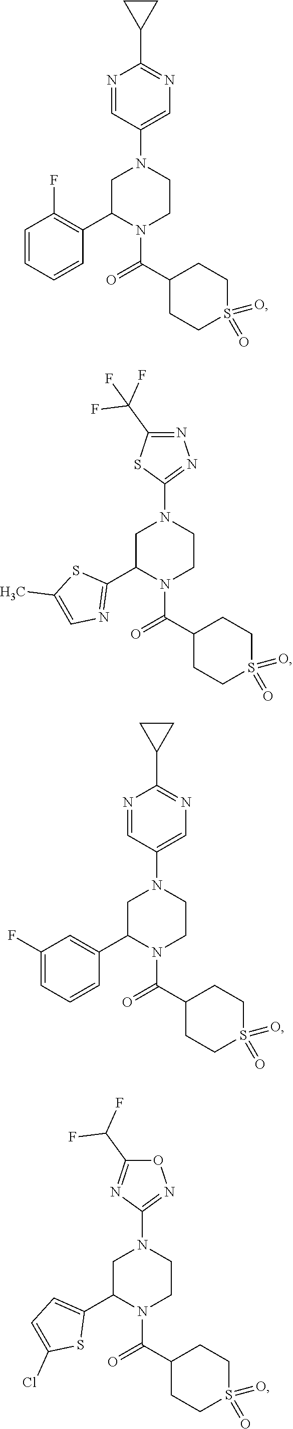 [SMILES: O=C(C1CCS(=O)(=O)CC1)N1CCN(c2cnc(C3CC3)nc2)CC1c1ccccc1F ; Cc1cnc(C2CN(c3nnc(C(F)(F)F)s3)CCN2C(=O)C2CCS(=O)(=O)CC2)s1 ; O=C(C1CCS(=O)(=O)CC1)N1CCN(c2cnc(C3CC3)nc2)CC1c1cccc(F)c1 ; O=C(C1CCS(=O)(=O)CC1)N1CCN(c2noc(C(F)F)n2)CC1c1ccc(Cl)s1]

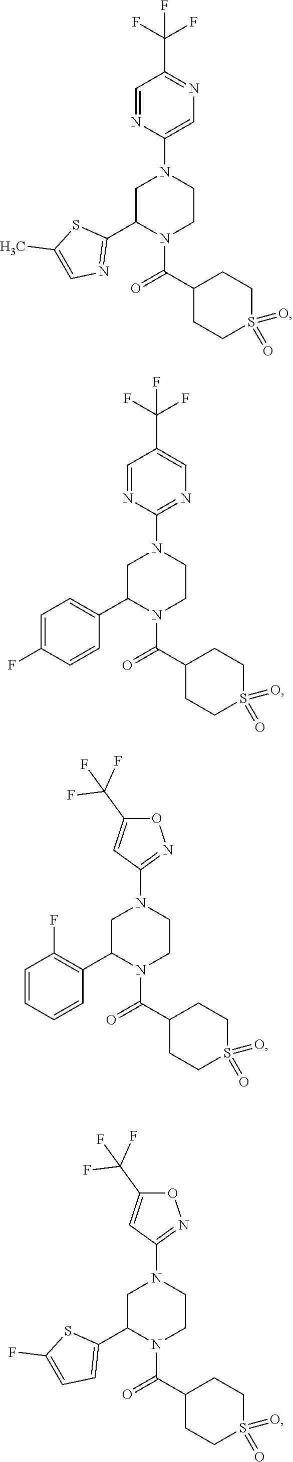 [SMILES: Cc1cnc(C2CN(c3cnc(C(F)(F)F)cn3)CCN2C(=O)C2CCS(=O)(=O)CC2)s1 ; O=C(C1CCS(=O)(=O)CC1)N1CCN(c2ncc(C(F)(F)F)cn2)CC1c1ccc(F)cc1 ; O=C(C1CCS(=O)(=O)CC1)N1CCN(c2cc(C(F)(F)F)on2)CC1c1ccccc1F ; O=C(C1CCS(=O)(=O)CC1)N1CCN(c2cc(C(F)(F)F)on2)CC1c1ccc(F)s1]

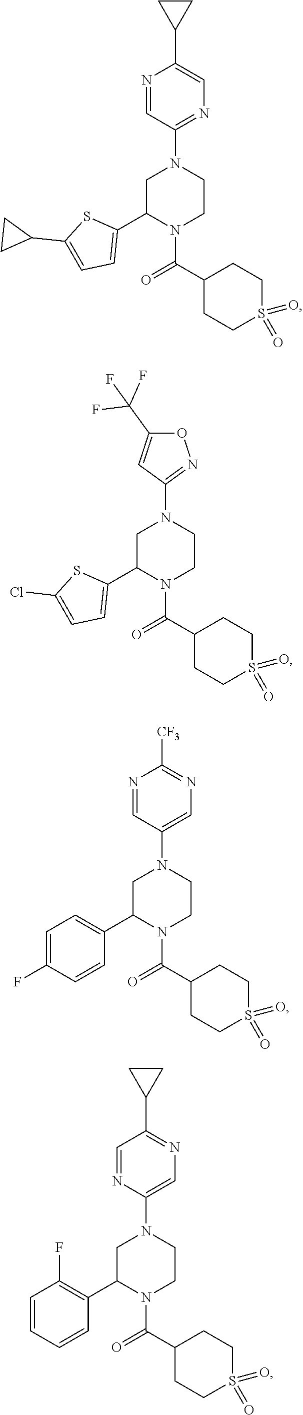 [SMILES: O=C(C1CCS(=O)(=O)CC1)N1CCN(c2cnc(C3CC3)cn2)CC1c1ccc(C2CC2)s1 ; O=C(C1CCS(=O)(=O)CC1)N1CCN(c2cc(C(F)(F)F)on2)CC1c1ccc(Cl)s1 ; O=C(C1CCS(=O)(=O)CC1)N1CCN(c2cnc(C(F)(F)F)nc2)CC1c1ccc(F)cc1 ; O=C(C1CCS(=O)(=O)CC1)N1CCN(c2cnc(C3CC3)cn2)CC1c1ccccc1F]

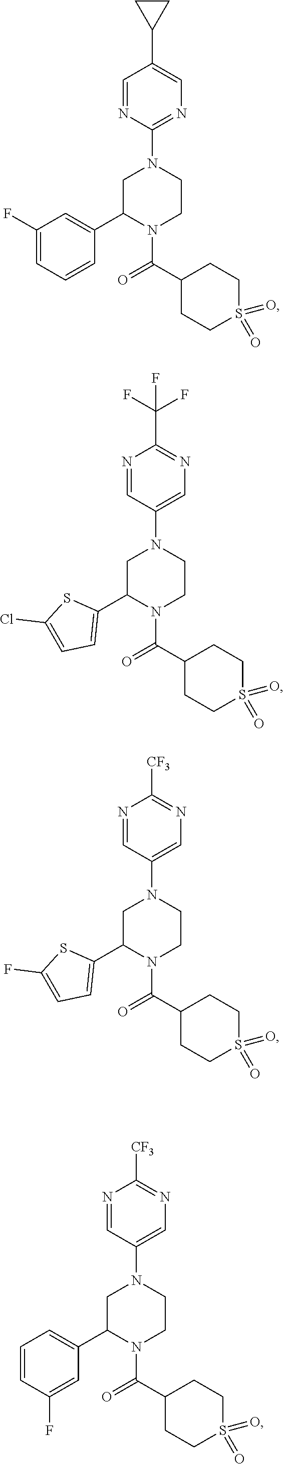 [SMILES: O=C(C1CCS(=O)(=O)CC1)N1CCN(c2ncc(C3CC3)cn2)CC1c1cccc(F)c1 ; O=C(C1CCS(=O)(=O)CC1)N1CCN(c2cnc(C(F)(F)F)nc2)CC1c1ccc(Cl)s1 ; O=C(C1CCS(=O)(=O)CC1)N1CCN(c2cnc(C(F)(F)F)nc2)CC1c1ccc(F)s1 ; O=C(C1CCS(=O)(=O)CC1)N1CCN(c2cnc(C(F)(F)F)nc2)CC1c1cccc(F)c1]

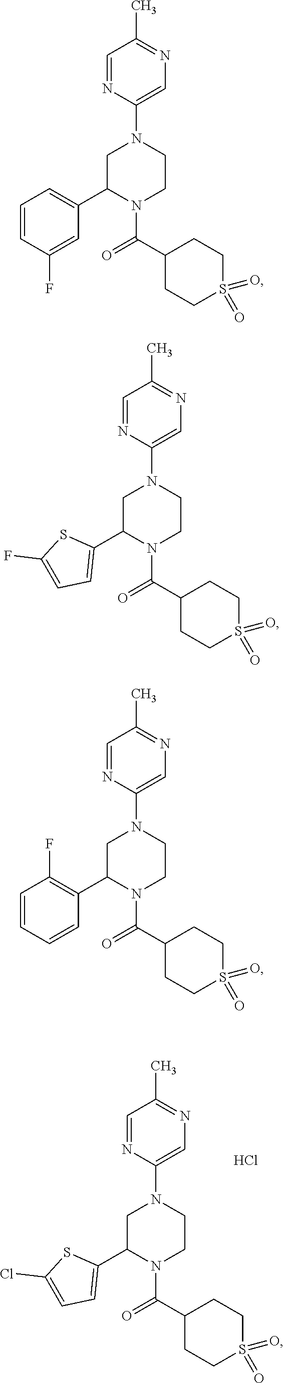 [SMILES: Cc1cnc(N2CCN(C(=O)C3CCS(=O)(=O)CC3)C(c3cccc(F)c3)C2)cn1 ; Cc1cnc(N2CCN(C(=O)C3CCS(=O)(=O)CC3)C(c3ccc(F)s3)C2)cn1 ; Cc1cnc(N2CCN(C(=O)C3CCS(=O)(=O)CC3)C(c3ccccc3F)C2)cn1 ; Cc1cnc(N2CCN(C(=O)C3CCS(=O)(=O)CC3)C(c3ccc(Cl)s3)C2)cn1 ; Cl]

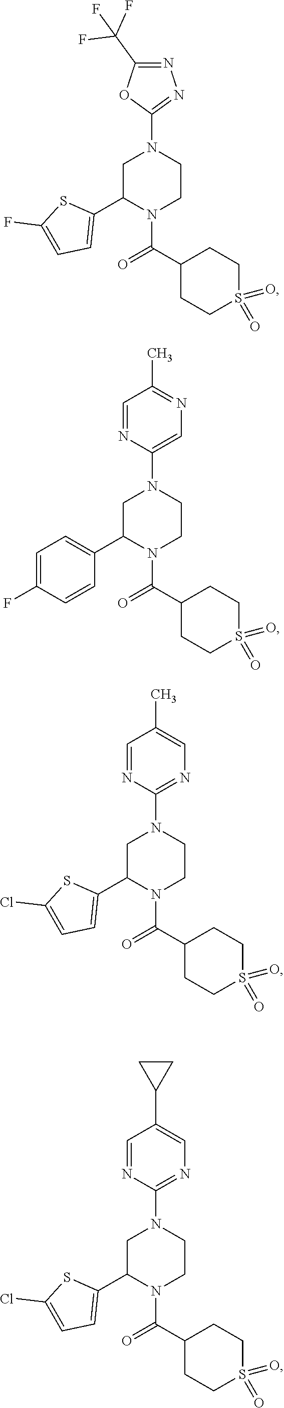 [SMILES: O=C(C1CCS(=O)(=O)CC1)N1CCN(c2nnc(C(F)(F)F)o2)CC1c1ccc(F)s1 ; Cc1cnc(N2CCN(C(=O)C3CCS(=O)(=O)CC3)C(c3ccc(F)cc3)C2)cn1 ; Cc1cnc(N2CCN(C(=O)C3CCS(=O)(=O)CC3)C(c3ccc(Cl)s3)C2)nc1 ; O=C(C1CCS(=O)(=O)CC1)N1CCN(c2ncc(C3CC3)cn2)CC1c1ccc(Cl)s1]

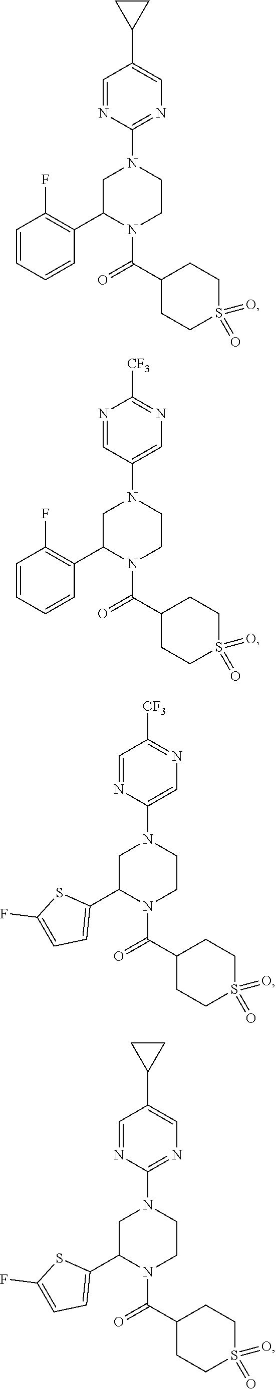 [SMILES: O=C(C1CCS(=O)(=O)CC1)N1CCN(c2ncc(C3CC3)cn2)CC1c1ccccc1F ; O=C(C1CCS(=O)(=O)CC1)N1CCN(c2cnc(C(F)(F)F)nc2)CC1c1ccccc1F ; O=C(C1CCS(=O)(=O)CC1)N1CCN(c2cnc(C(F)(F)F)cn2)CC1c1ccc(F)s1 ; O=C(C1CCS(=O)(=O)CC1)N1CCN(c2ncc(C3CC3)cn2)CC1c1ccc(F)s1]

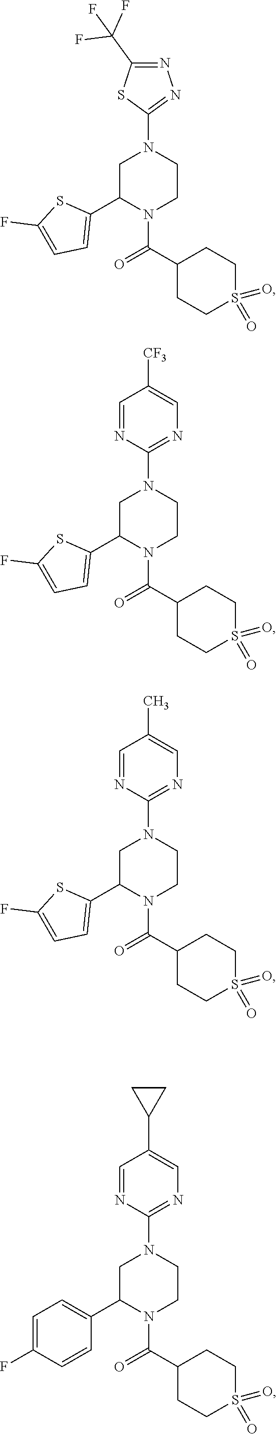 [SMILES: O=C(C1CCS(=O)(=O)CC1)N1CCN(c2nnc(C(F)(F)F)s2)CC1c1ccc(F)s1 ; O=C(C1CCS(=O)(=O)CC1)N1CCN(c2ncc(C(F)(F)F)cn2)CC1c1ccc(F)s1 ; Cc1cnc(N2CCN(C(=O)C3CCS(=O)(=O)CC3)C(c3ccc(F)s3)C2)nc1 ; O=C(C1CCS(=O)(=O)CC1)N1CCN(c2ncc(C3CC3)cn2)CC1c1ccc(F)cc1]

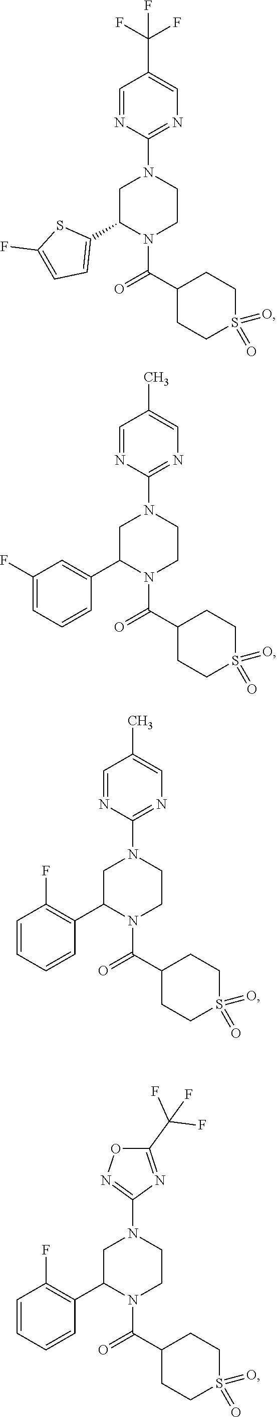 [SMILES: O=C(C1CCS(=O)(=O)CC1)N1CCN(c2ncc(C(F)(F)F)cn2)C[C@H]1c1ccc(F)s1 ; Cc1cnc(N2CCN(C(=O)C3CCS(=O)(=O)CC3)C(c3cccc(F)c3)C2)nc1 ; Cc1cnc(N2CCN(C(=O)C3CCS(=O)(=O)CC3)C(c3ccccc3F)C2)nc1 ; O=C(C1CCS(=O)(=O)CC1)N1CCN(c2noc(C(F)(F)F)n2)CC1c1ccccc1F]

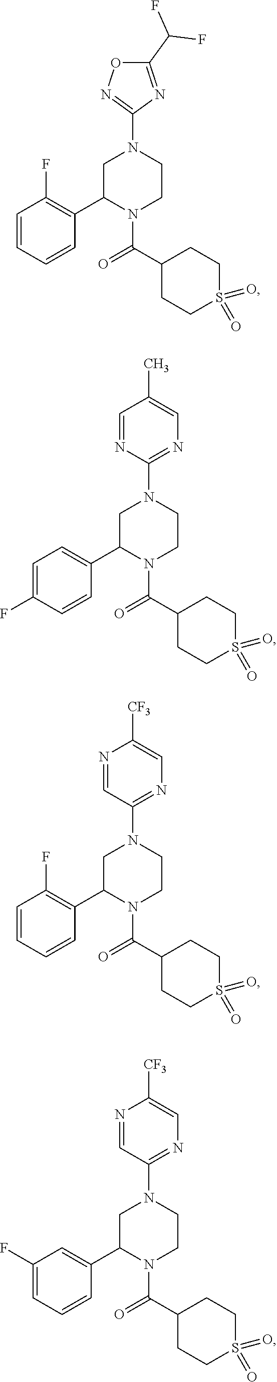 [SMILES: O=C(C1CCS(=O)(=O)CC1)N1CCN(c2noc(C(F)F)n2)CC1c1ccccc1F ; Cc1cnc(N2CCN(C(=O)C3CCS(=O)(=O)CC3)C(c3ccc(F)cc3)C2)nc1 ; O=C(C1CCS(=O)(=O)CC1)N1CCN(c2cnc(C(F)(F)F)cn2)CC1c1ccccc1F ; O=C(C1CCS(=O)(=O)CC1)N1CCN(c2cnc(C(F)(F)F)cn2)CC1c1cccc(F)c1]

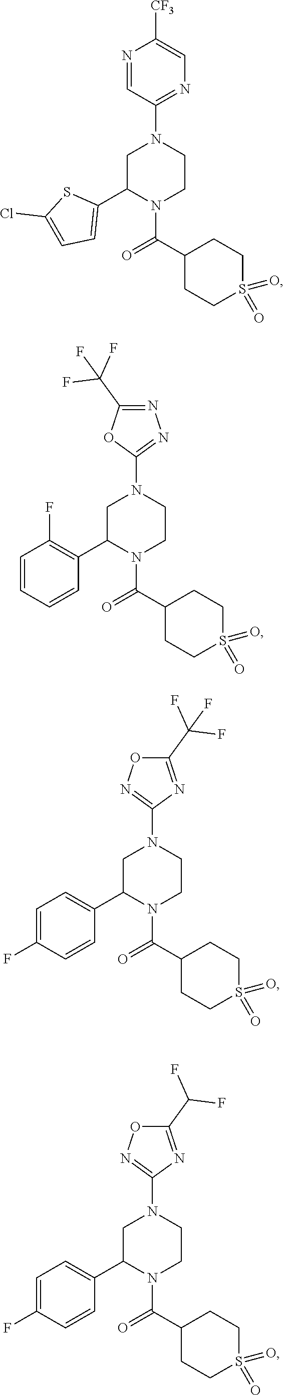

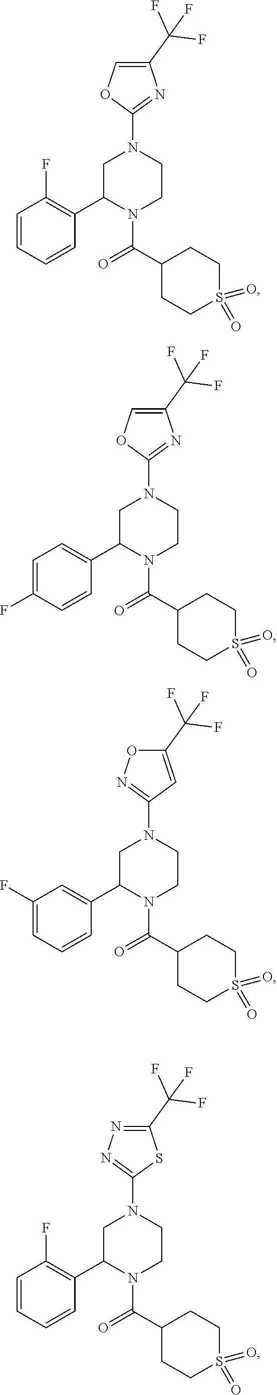

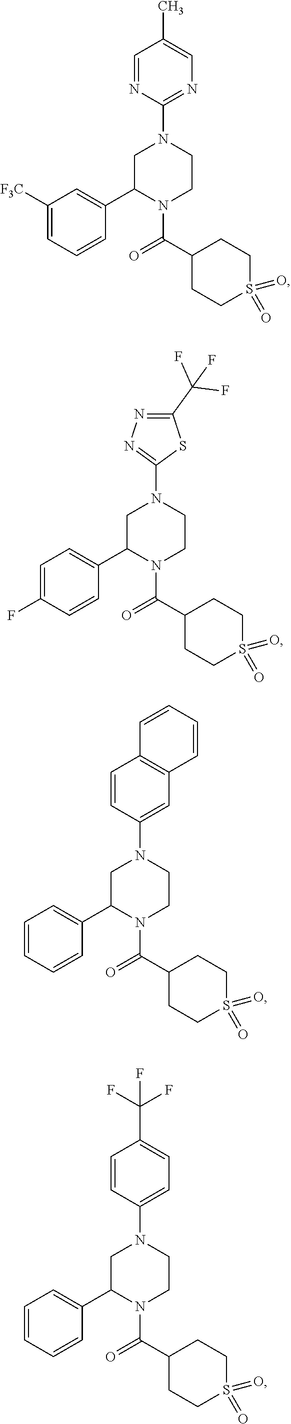 [SMILES: Cc1cnc(N2CCN(C(=O)C3CCS(=O)(=O)CC3)C(c3cccc(C(F)(F)F)c3)C2)nc1 ; O=C(C1CCS(=O)(=O)CC1)N1CCN(c2nnc(C(F)(F)F)s2)CC1c1ccc(F)cc1 ; O=C(C1CCS(=O)(=O)CC1)N1CCN(c2ccc3ccccc3c2)CC1c1ccccc1 ; O=C(C1CCS(=O)(=O)CC1)N1CCN(c2ccc(C(F)(F)F)cc2)CC1c1ccccc1]

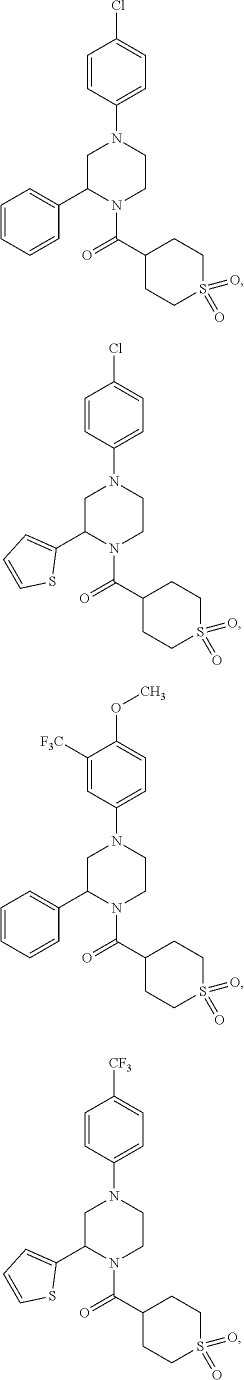 [SMILES: O=C(C1CCS(=O)(=O)CC1)N1CCN(c2ccc(Cl)cc2)CC1c1ccccc1 ; O=C(C1CCS(=O)(=O)CC1)N1CCN(c2ccc(Cl)cc2)CC1c1cccs1 ; COc1ccc(N2CCN(C(=O)C3CCS(=O)(=O)CC3)C(c3ccccc3)C2)cc1C(F)(F)F ; O=C(C1CCS(=O)(=O)CC1)N1CCN(c2ccc(C(F)(F)F)cc2)CC1c1cccs1]

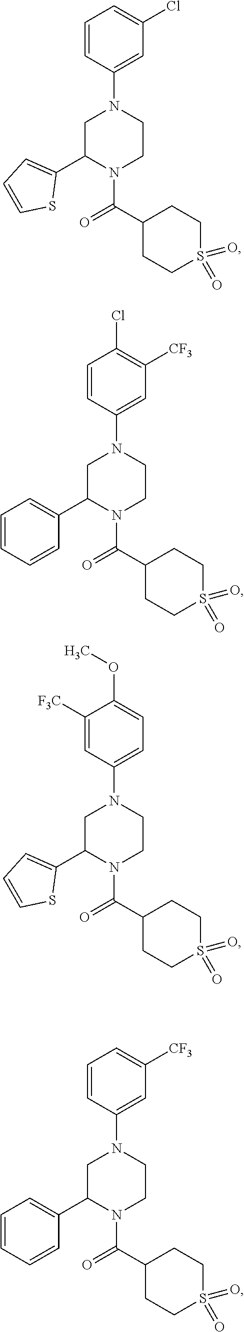 [SMILES: O=C(C1CCS(=O)(=O)CC1)N1CCN(c2cccc(Cl)c2)CC1c1cccs1 ; O=C(C1CCS(=O)(=O)CC1)N1CCN(c2ccc(Cl)c(C(F)(F)F)c2)CC1c1ccccc1 ; COc1ccc(N2CCN(C(=O)C3CCS(=O)(=O)CC3)C(c3cccs3)C2)cc1C(F)(F)F ; O=C(C1CCS(=O)(=O)CC1)N1CCN(c2cccc(C(F)(F)F)c2)CC1c1ccccc1]

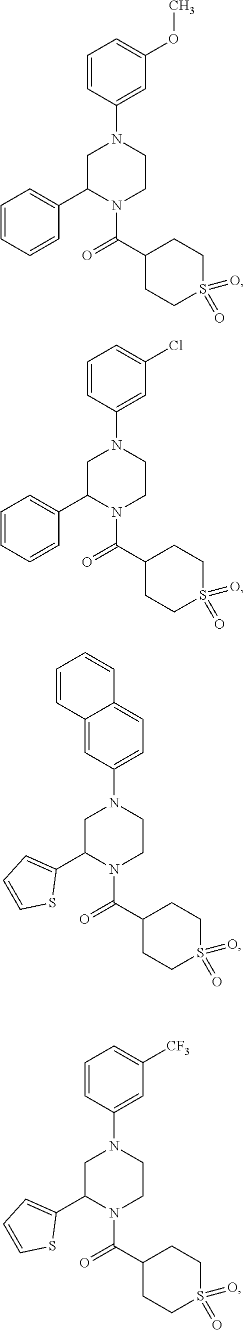

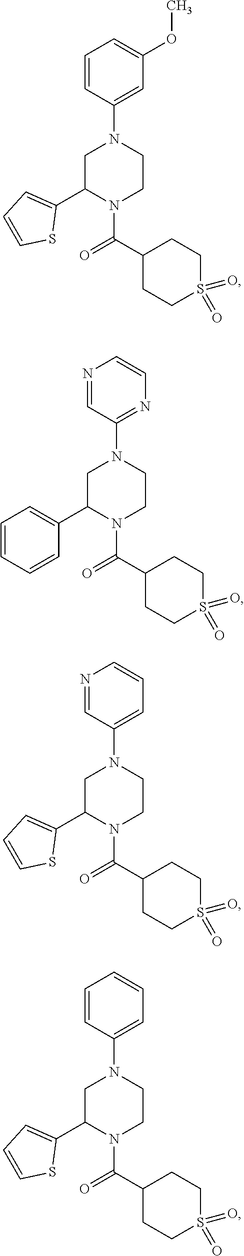 [SMILES: COc1cccc(N2CCN(C(=O)C3CCS(=O)(=O)CC3)C(c3cccs3)C2)c1 ; O=C(C1CCS(=O)(=O)CC1)N1CCN(c2cnccn2)CC1c1ccccc1 ; O=C(C1CCS(=O)(=O)CC1)N1CCN(c2cccnc2)CC1c1cccs1 ; O=C(C1CCS(=O)(=O)CC1)N1CCN(c2ccccc2)CC1c1cccs1]

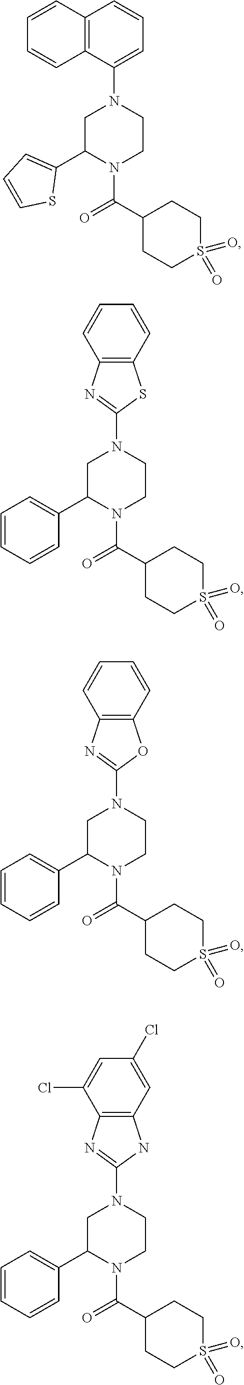 [SMILES: O=C(C1CCS(=O)(=O)CC1)N1CCN(c2cccc3ccccc23)CC1c1cccs1 ; O=C(C1CCS(=O)(=O)CC1)N1CCN(c2nc3ccccc3s2)CC1c1ccccc1 ; O=C(C1CCS(=O)(=O)CC1)N1CCN(c2nc3ccccc3o2)CC1c1ccccc1 ; O=C(C1CCS(=O)(=O)CC1)N1CCN(c2nc3c(Cl)cc(Cl)cc3[nH]2)CC1c1ccccc1]

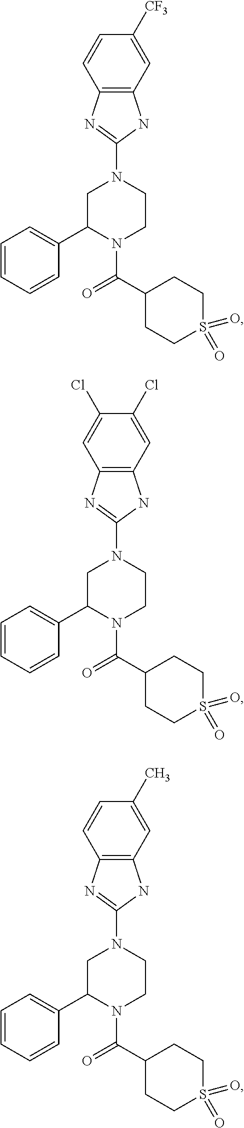 [SMILES: O=C(C1CCS(=O)(=O)CC1)N1CCN(c2nc3ccc(C(F)(F)F)cc3[nH]2)CC1c1ccccc1 ; O=C(C1CCS(=O)(=O)CC1)N1CCN(c2nc3cc(Cl)c(Cl)cc3[nH]2)CC1c1ccccc1 ; Cc1ccc2nc(N3CCN(C(=O)C4CCS(=O)(=O)CC4)C(c4ccccc4)C3)[nH]c2c1]

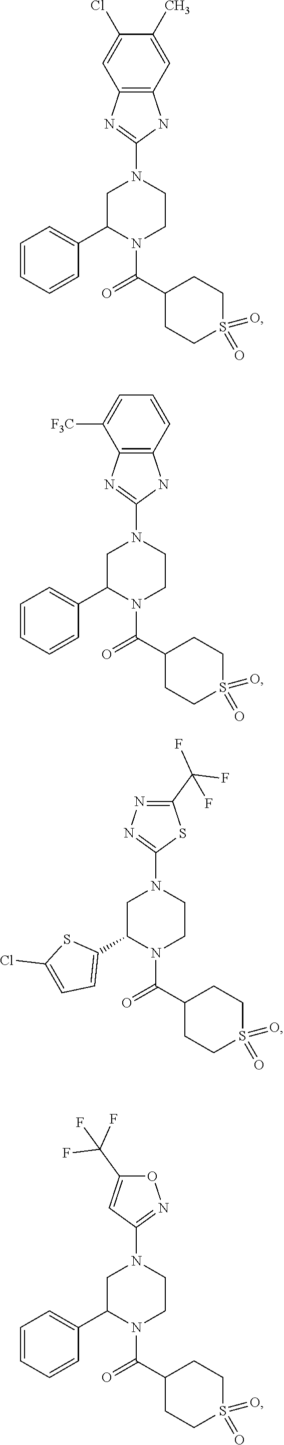

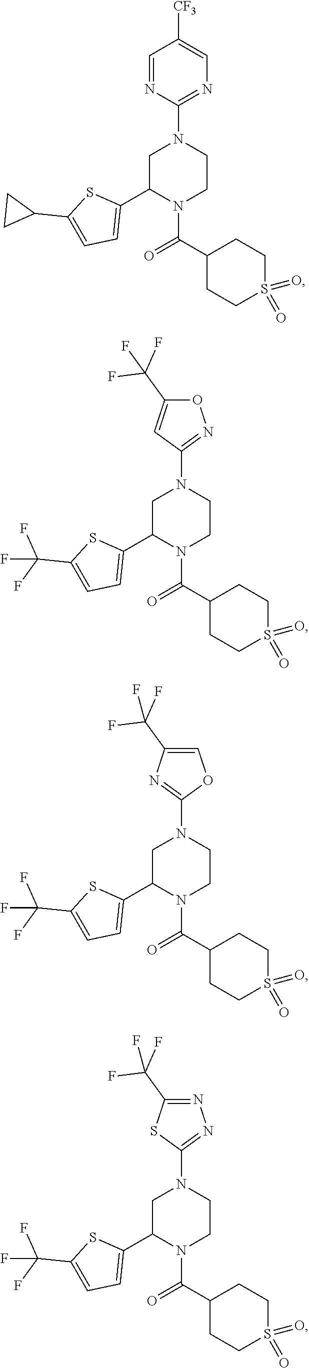 [SMILES: O=C(C1CCS(=O)(=O)CC1)N1CCN(c2ncc(C(F)(F)F)cn2)CC1c1ccc(C2CC2)s1 ; O=C(C1CCS(=O)(=O)CC1)N1CCN(c2cc(C(F)(F)F)on2)CC1c1ccc(C(F)(F)F)s1 ; O=C(C1CCS(=O)(=O)CC1)N1CCN(c2nc(C(F)(F)F)co2)CC1c1ccc(C(F)(F)F)s1 ; O=C(C1CCS(=O)(=O)CC1)N1CCN(c2nnc(C(F)(F)F)s2)CC1c1ccc(C(F)(F)F)s1]

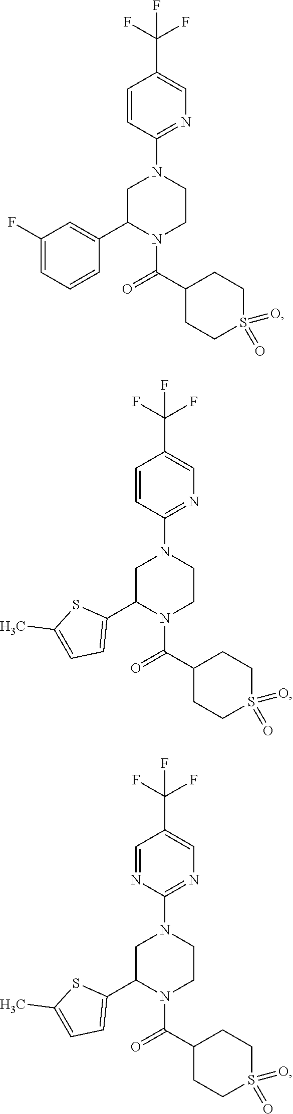 [SMILES: O=C(C1CCS(=O)(=O)CC1)N1CCN(c2ccc(C(F)(F)F)cn2)CC1c1cccc(F)c1 ; Cc1ccc(C2CN(c3ccc(C(F)(F)F)cn3)CCN2C(=O)C2CCS(=O)(=O)CC2)s1 ; Cc1ccc(C2CN(c3ncc(C(F)(F)F)cn3)CCN2C(=O)C2CCS(=O)(=O)CC2)s1]

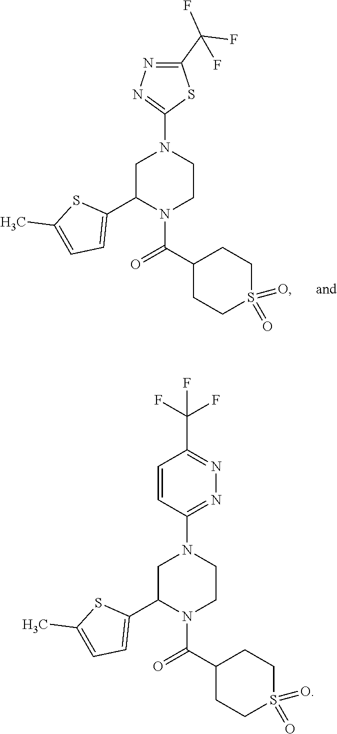 [SMILES: Cc1ccc(C2CN(c3nnc(C(F)(F)F)s3)CCN2C(=O)C2CCS(=O)(=O)CC2)s1 ; "and" ; Cc1ccc(C2CN(c3ccc(C(F)(F)F)nn3)CCN2C(=O)C2CCS(=O)(=O)CC2)s1]

In some embodiments of the methods and uses disclosed herein, the GlyT1 inhibitor is a compound of Formula X,

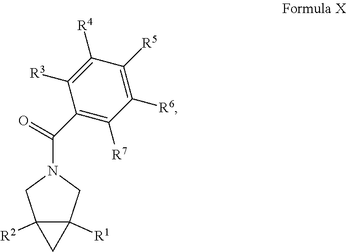

wherein:
R¹ is selected from the group consisting of
a) 5 or 6 membered monocyclic heteroaryl, having 1, 2, 3 or 4 heteroatoms independently selected from the group consisting of O, N and S(O)r,
b) 5 or 6 membered monocyclic partially saturated heterocycloalkyl, having 1, 2 or 3 heteroatoms independently selected from the group consisting of O, N and S(O)r, and
c) 9 or 10 membered bicyclic heteroaryl, having 1, 2 or 3 heteroatoms independently selected from the group consisting of O, N and S(O)ᵣ,
wherein r is 0, 1 or 2;
wherein each of said groups a), b) and c) is optionally substituted with 1 or more substituents independently selected from the group consisting of C₁₋₄-alkyl-, C₁₋₄-alkyl-O—, oxetanyl, tetrahydrofuranyl, tetrahydropyranyl, C₃₋₆-cycloalkyl- and C₃₋₆-cycloalkyl-O— and in case a substituent is attached to a nitrogen ring atom said substituent is selected from the group consisting of C₁₋₄-alkyl-, C₁₋₄-alkyl-CO—, C₃₋₆-cycloalkyl- and C₃₋₆-cycloalkyl-CO—,
and wherein each of said C₁₋₄-alkyl-, C₁₋₄-alkyl-O—, C₁₋₄-alkyl-CO—, oxetanyl, tetrahydrofuranyl, tetrahydropyranyl, C₃₋₆-cycloalkyl-, C₃₋₆-cycloalkyl-CO— or C₃₋₆-cycloalkyl-O— substituents may be substituted by 1 or more substituents independently selected from the group consisting of fluoro, —CF₃, —CHF₂, —CH₂F and —CN;
R² is selected from the group consisting of hydrogen, C₁₋₄-alkyl-, C₁₋₄-alkyl-O—, —CN and C₃₋₆-cycloalkyl-,
wherein each of said C₁₋₄-alkyl-, C₁₋₄-alkyl-O— and C₃₋₆-cycloalkyl-group may be optionally substituted with 1, 2, 3 or more substituents independently selected from the group consisting of fluoro, —CF₃, —CHF₂, —CH₂F and —CN;
R³ is selected from the group consisting of C₁₋₆-alkyl-O—, C₃₋₆-cycloalkyl-O—, morpholino, pyrazolyl and a 4 to 7 membered, monocyclic heterocycloalkyl-O— with 1 oxygen atom as ring member and optionally 1 or 2 heteroatoms independently selected from the group consisting of O, N and S(O)ₛ with s=0, 1 or 2,
wherein said C₁₋₆-alkyl-O— and said C₃₋₆-cycloalkyl-O— may be optionally substituted with 1, 2, 3 or more substituents independently selected from the group consisting of fluoro, —CF₃, —CHF₂, —CH₂F, —CN, C₁₋₄-alkyl-, C₃₋₆-cycloalkyl-, C₁₋₆-alkyl-O- and C₃₋₆-cycloalkyl-O—;
R⁴ is hydrogen;
or R³ and R⁴ together with the ring atoms of the phenyl group to which they are bound may form a 4, 5 or 6 membered, monocyclic, partially saturated heterocycloalkyl or a heteroaryl each of which having 1, 2 or 3 heteroatoms independently selected from the group consisting of O, N and S(O)ₛ with s=0, 1 or 2, wherein there must be 1 ring oxygen atom that is directly attached to the ring carbon atom of said phenyl group to which R³ is attached to in general formula (I);
wherein said heterocycloalkyl group may be optionally substituted with 1, 2, 3 or more substituents independently selected from the group consisting of fluoro, —CF₃, —CHF₂, —CH₂F, —CN, C₁₋₄-alkyl-, C₃₋₆-cycloalkyl-, C₁₋₆-alkyl-O—, C₃₋₆-cycloalkyl-O—, oxetanyl-O—, tetrahydrofuranyl-O— and tetrahydropyranyl-O—;
R⁵ is hydrogen;
R⁶ is selected from the group consisting of hydrogen, C₁₋₄-alkyl-SO₂—, C₃₋₆-cycloalkyl-SO₂ and —CN;
R⁷ is hydrogen;
or one of the pairs a) R⁶ and R⁷ or b) R⁶ and R⁵ form together with the ring atoms of the phenyl group to which they are bound, a 5 or 6 membered, partially saturated monocyclic heterocycloalkyl group having 1, 2 or 3 heteroatoms independently selected from the group consisting of O, N and S(O)ᵤ with u=0, 1 or 2, wherein there must be 1 —SO₂— member that is directly attached to the ring carbon atom of said phenyl group to which R⁶ is attached to in general formula (I),
wherein said heterocycloalkyl group may be optionally substituted with 1, 2, 3 or more substituents independently selected from the group consisting of fluoro, —CF₃, —CHF₂, —CH₂F, —CN, C₁₋₄-alkyl-, C₁₋₆-alkyl-O— and C₃₋₆-cycloalkyl-O—
or a pharmaceutically acceptable salt thereof, or a prodrug of the compound or its pharmaceutically acceptable salt.

In certain embodiments, the compound of formula X is a compound selected from any of the following, a stereoisomer or stereoisomeric mixture thereof, or a pharmaceutically acceptable salt thereof:

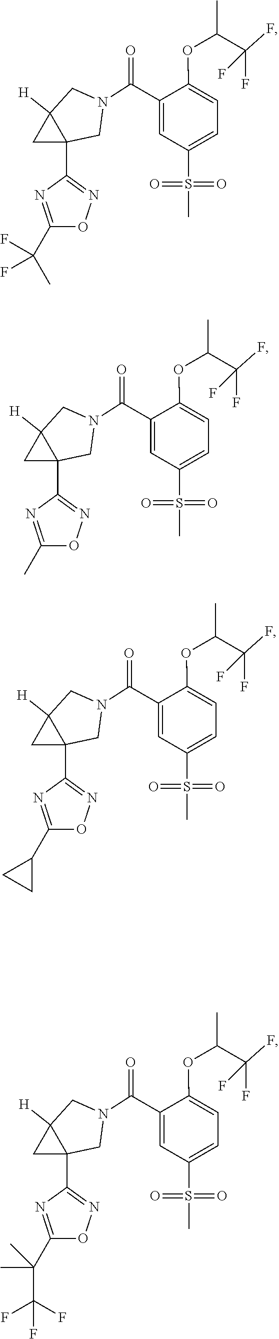 [SMILES: CC(Oc1ccc(S(C)(=O)=O)cc1C(=O)N1CC2CC2(c2noc(C(C)(F)F)n2)C1)C(F)(F)F ; Cc1nc(C23CC2CN(C(=O)c2cc(S(C)(=O)=O)ccc2OC(C)C(F)(F)F)C3)no1 ; CC(Oc1ccc(S(C)(=O)=O)cc1C(=O)N1CC2CC2(c2noc(C3CC3)n2)C1)C(F)(F)F ; CC(Oc1ccc(S(C)(=O)=O)cc1C(=O)N1CC2CC2(c2noc(C(C)(C)C(F)(F)F)n2)C1)C(F)(F)F]

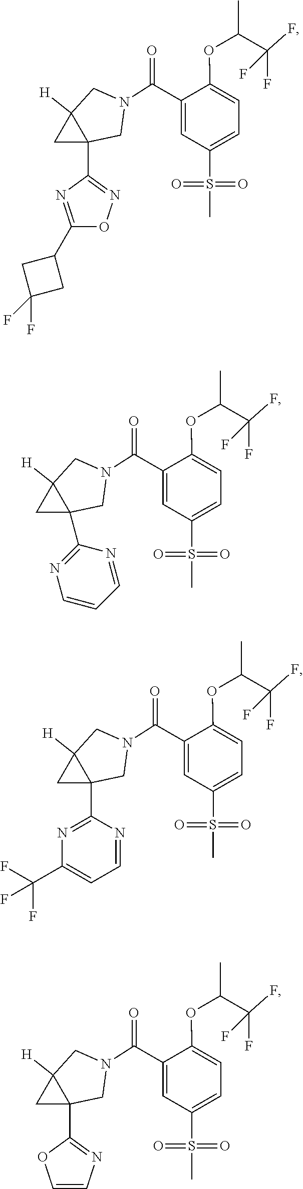 [SMILES: CC(Oc1ccc(S(C)(=O)=O)cc1C(=O)N1CC2CC2(c2noc(C3CC(F)(F)C3)n2)C1)C(F)(F)F ; CC(Oc1ccc(S(C)(=O)=O)cc1C(=O)N1CC2CC2(c2ncccn2)C1)C(F)(F)F ; CC(Oc1ccc(S(C)(=O)=O)cc1C(=O)N1CC2CC2(c2nccc(C(F)(F)F)n2)C1)C(F)(F)F ; CC(Oc1ccc(S(C)(=O)=O)cc1C(=O)N1CC2CC2(c2ncco2)C1)C(F)(F)F]

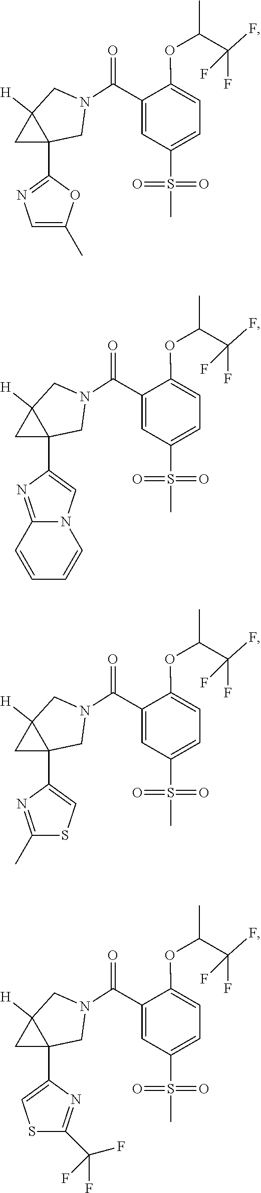 [SMILES: Cc1cnc(C23CC2CN(C(=O)c2cc(S(C)(=O)=O)ccc2OC(C)C(F)(F)F)C3)o1 ; CC(Oc1ccc(S(C)(=O)=O)cc1C(=O)N1CC2CC2(c2cn3ccccc3n2)C1)C(F)(F)F ; Cc1nc(C23CC2CN(C(=O)c2cc(S(C)(=O)=O)ccc2OC(C)C(F)(F)F)C3)cs1 ; CC(Oc1ccc(S(C)(=O)=O)cc1C(=O)N1CC2CC2(c2csc(C(F)(F)F)n2)C1)C(F)(F)F]

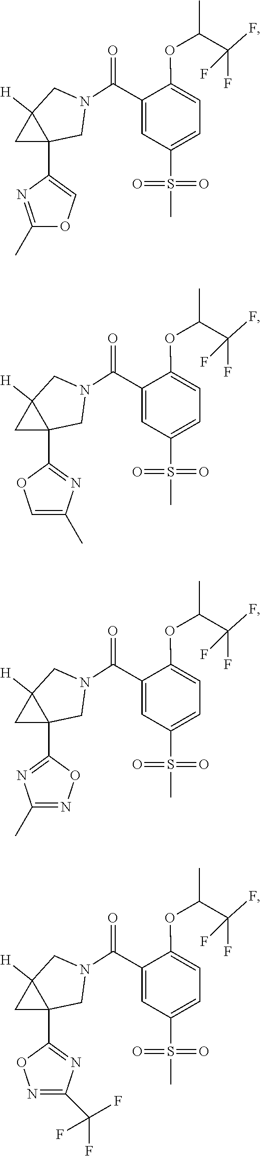 [SMILES: Cc1nc(C23CC2CN(C(=O)c2cc(S(C)(=O)=O)ccc2OC(C)C(F)(F)F)C3)co1 ; Cc1coc(C23CC2CN(C(=O)c2cc(S(C)(=O)=O)ccc2OC(C)C(F)(F)F)C3)n1 ; Cc1noc(C23CC2CN(C(=O)c2cc(S(C)(=O)=O)ccc2OC(C)C(F)(F)F)C3)n1 ; CC(Oc1ccc(S(C)(=O)=O)cc1C(=O)N1CC2CC2(c2nc(C(F)(F)F)no2)C1)C(F)(F)F]

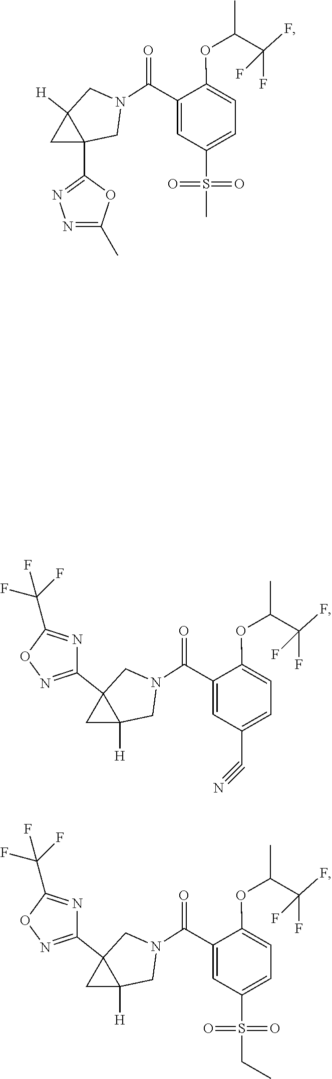 [SMILES: Cc1nnc(C23CC2CN(C(=O)c2cc(S(C)(=O)=O)ccc2OC(C)C(F)(F)F)C3)o1 ; CC(Oc1ccc(C#N)cc1C(=O)N1CC2CC2(c2noc(C(F)(F)F)n2)C1)C(F)(F)F ; CCS(=O)(=O)c1ccc(OC(C)C(F)(F)F)c(C(=O)N2CC3CC3(c3noc(C(F)(F)F)n3)C2)c1]

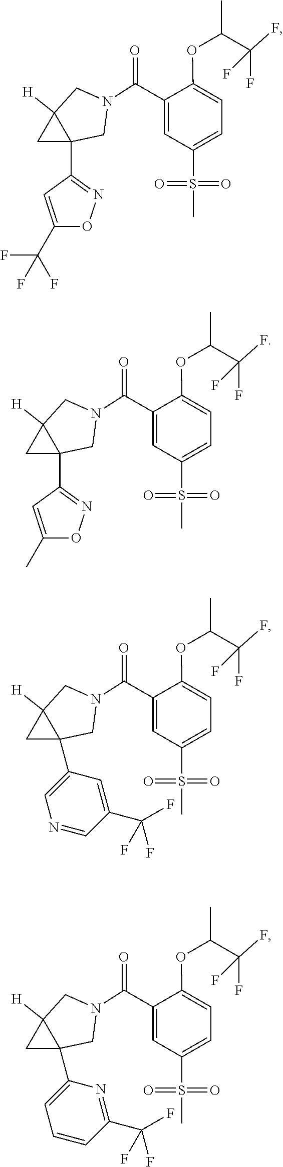 [SMILES: CC(Oc1ccc(S(C)(=O)=O)cc1C(=O)N1CC2CC2(c2cc(C(F)(F)F)on2)C1)C(F)(F)F ; Cc1cc(C23CC2CN(C(=O)c2cc(S(C)(=O)=O)ccc2OC(C)C(F)(F)F)C3)no1 ; CC(Oc1ccc(S(C)(=O)=O)cc1C(=O)N1CC2CC2(c2cncc(C(F)(F)F)c2)C1)C(F)(F)F ; CC(Oc1ccc(S(C)(=O)=O)cc1C(=O)N1CC2CC2(c2cccc(C(F)(F)F)n2)C1)C(F)(F)F]

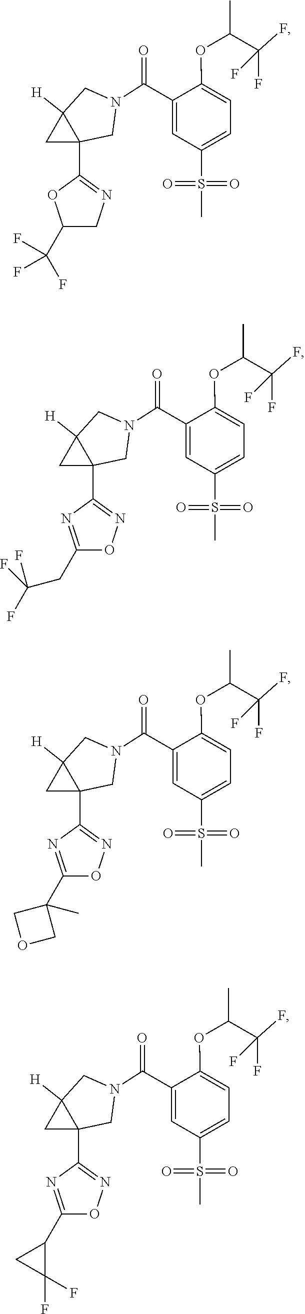 [SMILES: CC(Oc1ccc(S(C)(=O)=O)cc1C(=O)N1CC2CC2(C2=NCC(C(F)(F)F)O2)C1)C(F)(F)F ; CC(Oc1ccc(S(C)(=O)=O)cc1C(=O)N1CC2CC2(c2noc(CC(F)(F)F)n2)C1)C(F)(F)F ; CC(Oc1ccc(S(C)(=O)=O)cc1C(=O)N1CC2CC2(c2noc(C3(C)COC3)n2)C1)C(F)(F)F ; CC(Oc1ccc(S(C)(=O)=O)cc1C(=O)N1CC2CC2(c2noc(C3CC3(F)F)n2)C1)C(F)(F)F]

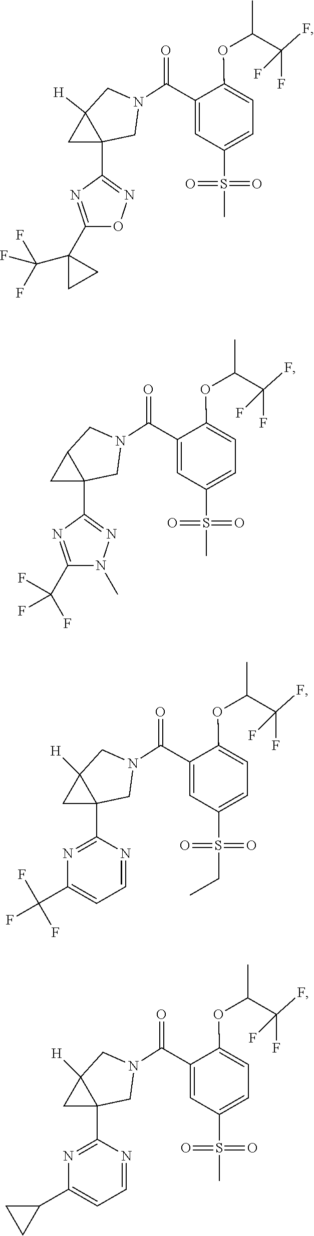 [SMILES: CC(Oc1ccc(S(C)(=O)=O)cc1C(=O)N1CC2CC2(c2noc(C3(C(F)(F)F)CC3)n2)C1)C(F)(F)F ; CC(Oc1ccc(S(C)(=O)=O)cc1C(=O)N1CC2CC2(c2nc(C(F)(F)F)n(C)n2)C1)C(F)(F)F ; CCS(=O)(=O)c1ccc(OC(C)C(F)(F)F)c(C(=O)N2CC3CC3(c3nccc(C(F)(F)F)n3)C2)c1 ; CC(Oc1ccc(S(C)(=O)=O)cc1C(=O)N1CC2CC2(c2nccc(C3CC3)n2)C1)C(F)(F)F]

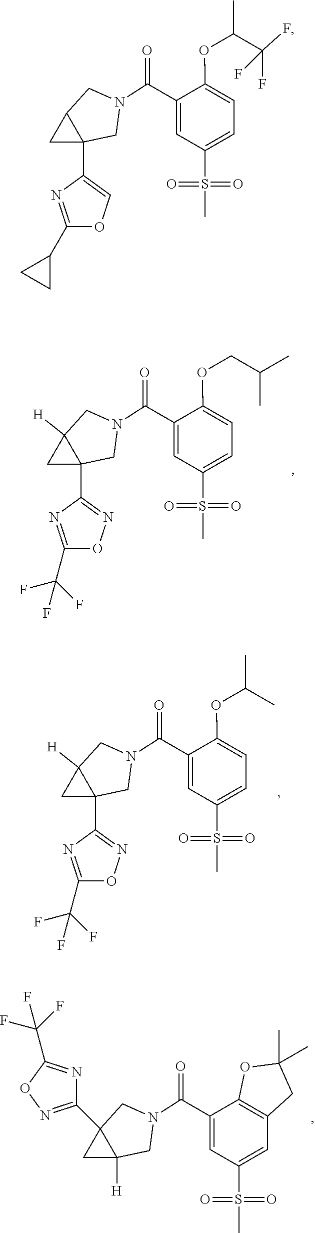 [SMILES: CC(Oc1ccc(S(C)(=O)=O)cc1C(=O)N1CC2CC2(c2coc(C3CC3)n2)C1)C(F)(F)F ; CC(C)COc1ccc(S(C)(=O)=O)cc1C(=O)N1CC2CC2(c2noc(C(F)(F)F)n2)C1 ; CC(C)Oc1ccc(S(C)(=O)=O)cc1C(=O)N1CC2CC2(c2noc(C(F)(F)F)n2)C1 ; CC1(C)Cc2cc(S(C)(=O)=O)cc(C(=O)N3CC4CC4(c4noc(C(F)(F)F)n4)C3)c2O1]

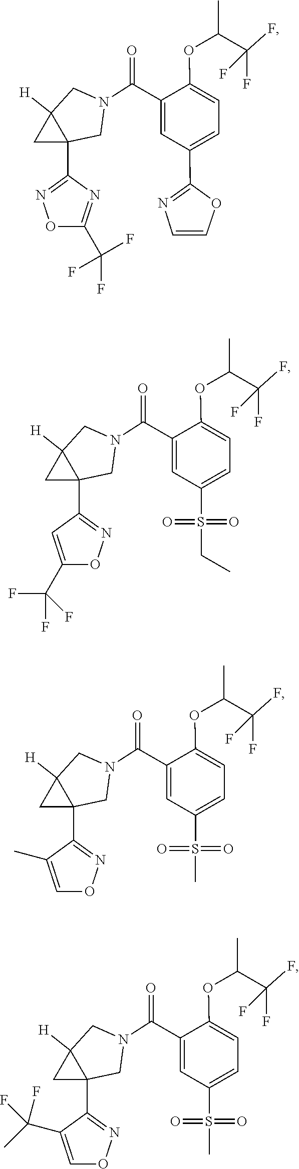 [SMILES: CC(Oc1ccc(-c2ncco2)cc1C(=O)N1CC2CC2(c2noc(C(F)(F)F)n2)C1)C(F)(F)F ; CCS(=O)(=O)c1ccc(OC(C)C(F)(F)F)c(C(=O)N2CC3CC3(c3cc(C(F)(F)F)on3)C2)c1 ; Cc1conc1C12CC1CN(C(=O)c1cc(S(C)(=O)=O)ccc1OC(C)C(F)(F)F)C2 ; CC(Oc1ccc(S(C)(=O)=O)cc1C(=O)N1CC2CC2(c2nocc2C(C)(F)F)C1)C(F)(F)F]

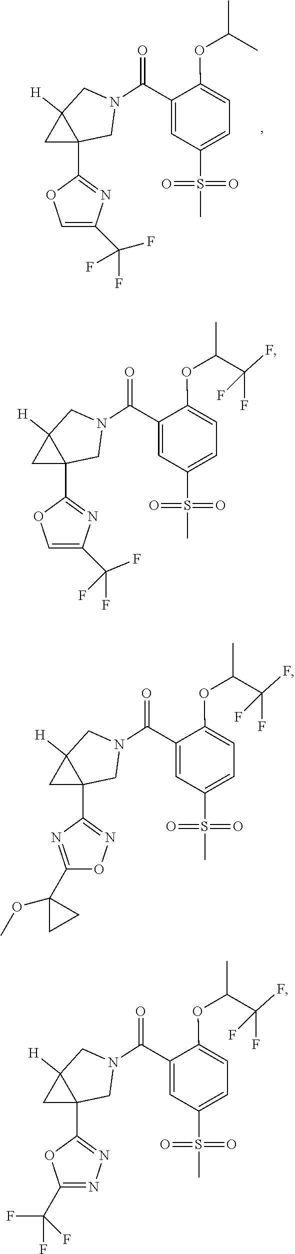

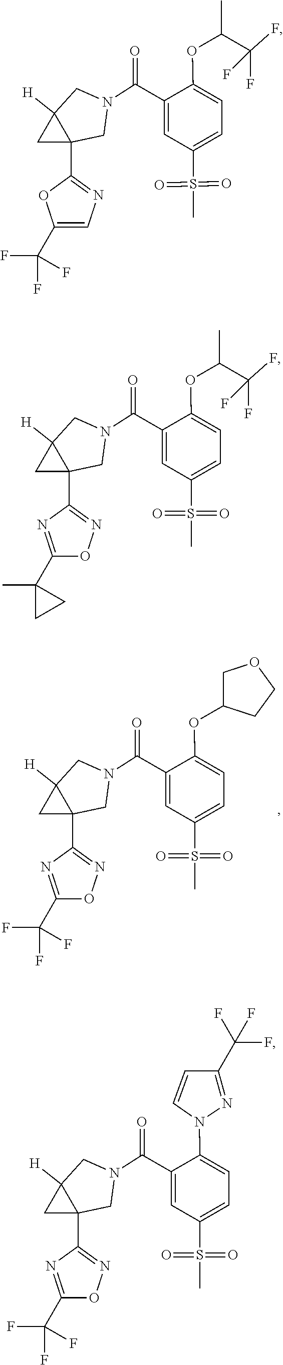 [SMILES: CC(Oc1ccc(S(C)(=O)=O)cc1C(=O)N1CC2CC2(c2ncc(C(F)(F)F)o2)C1)C(F)(F)F ; CC(Oc1ccc(S(C)(=O)=O)cc1C(=O)N1CC2CC2(c2noc(C3(C)CC3)n2)C1)C(F)(F)F ; CS(=O)(=O)c1ccc(OC2CCOC2)c(C(=O)N2CC3CC3(c3noc(C(F)(F)F)n3)C2)c1 ; CS(=O)(=O)c1ccc(-n2ccc(C(F)(F)F)n2)c(C(=O)N2CC3CC3(c3noc(C(F)(F)F)n3)C2)c1]

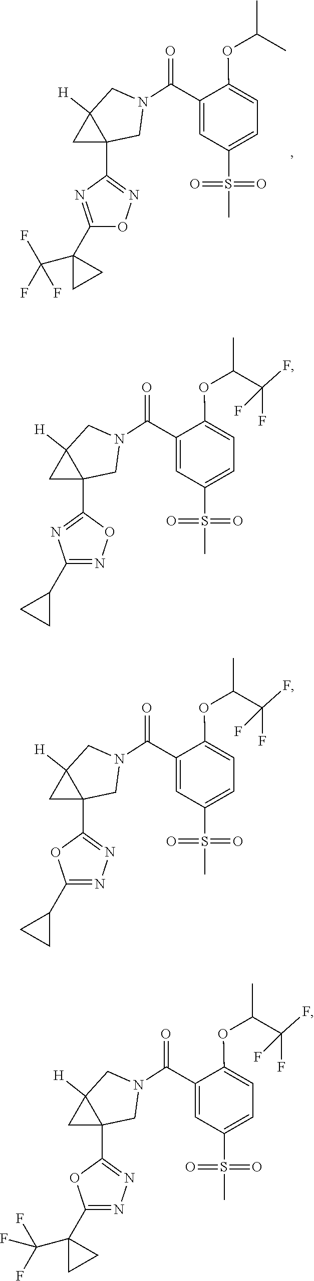 [SMILES: CC(C)Oc1ccc(S(C)(=O)=O)cc1C(=O)N1CC2CC2(c2noc(C3(C(F)(F)F)CC3)n2)C1 ; CC(Oc1ccc(S(C)(=O)=O)cc1C(=O)N1CC2CC2(c2nc(C3CC3)no2)C1)C(F)(F)F ; CC(Oc1ccc(S(C)(=O)=O)cc1C(=O)N1CC2CC2(c2nnc(C3CC3)o2)C1)C(F)(F)F ; CC(Oc1ccc(S(C)(=O)=O)cc1C(=O)N1CC2CC2(c2nnc(C3(C(F)(F)F)CC3)o2)C1)C(F)(F)F]

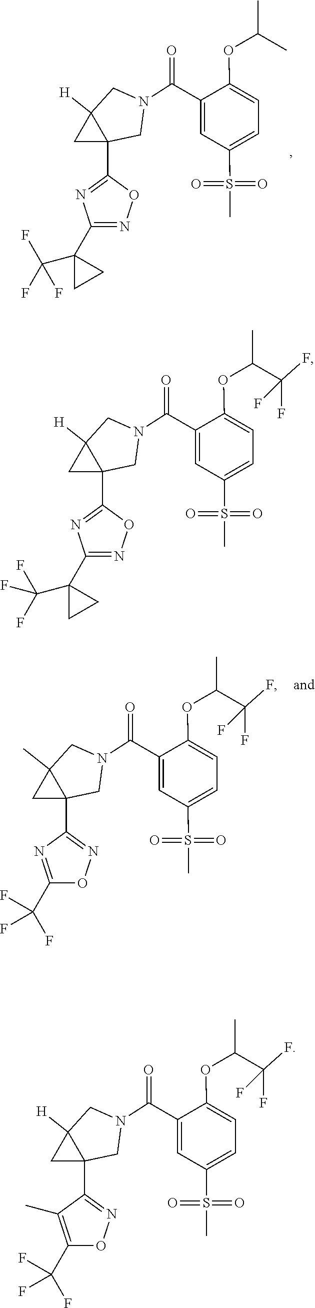 [SMILES: CC(C)Oc1ccc(S(C)(=O)=O)cc1C(=O)N1CC2CC2(c2nc(C3(C(F)(F)F)CC3)no2)C1 ; CC(Oc1ccc(S(C)(=O)=O)cc1C(=O)N1CC2CC2(c2nc(C3(C(F)(F)F)CC3)no2)C1)C(F)(F)F ; CC(Oc1ccc(S(C)(=O)=O)cc1C(=O)N1CC2(C)CC2(c2noc(C(F)(F)F)n2)C1)C(F)(F)F ; Cc1c(C23CC2CN(C(=O)c2cc(S(C)(=O)=O)ccc2OC(C)C(F)(F)F)C3)noc1C(F)(F)F]

For example, the compound of Formula X could be a diastereomeric mixture or single diastereomer of any of the following, or a pharmaceutically acceptable salt thereof:

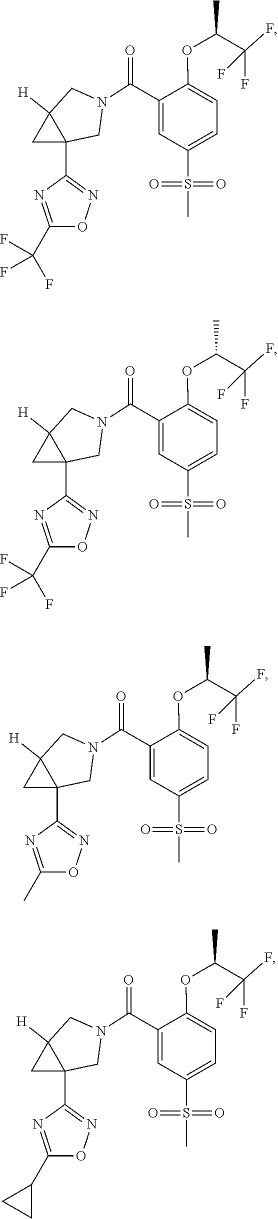 [SMILES: C[C@H](Oc1ccc(S(C)(=O)=O)cc1C(=O)N1CC2CC2(c2noc(C(F)(F)F)n2)C1)C(F)(F)F ; C[C@@H](Oc1ccc(S(C)(=O)=O)cc1C(=O)N1CC2CC2(c2noc(C(F)(F)F)n2)C1)C(F)(F)F ; Cc1nc(C23CC2CN(C(=O)c2cc(S(C)(=O)=O)ccc2O[C@@H](C)C(F)(F)F)C3)no1 ; C[C@H](Oc1ccc(S(C)(=O)=O)cc1C(=O)N1CC2CC2(c2noc(C3CC3)n2)C1)C(F)(F)F]

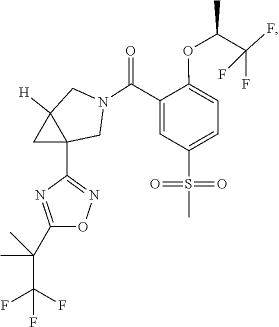 [SMILES: C[C@H](Oc1ccc(S(C)(=O)=O)cc1C(=O)N1CC2CC2(c2noc(C(C)(C)C(F)(F)F)n2)C1)C(F)(F)F]

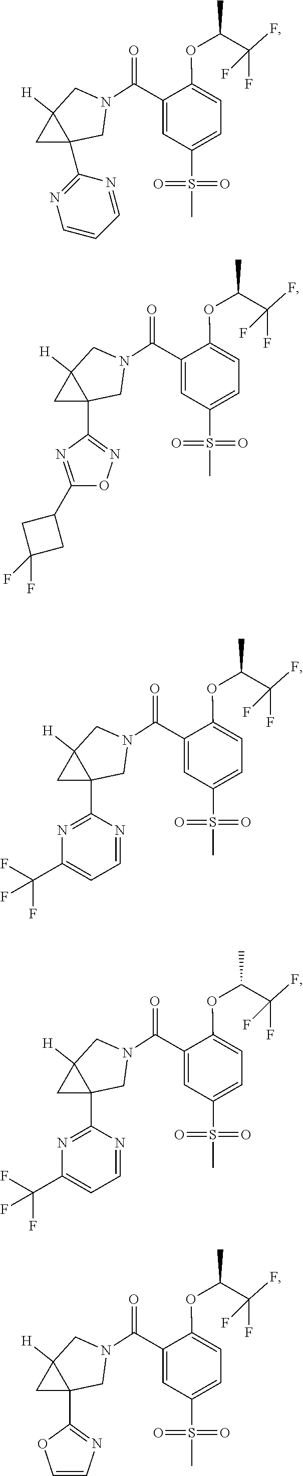 [SMILES: C[C@H](Oc1ccc(S(C)(=O)=O)cc1C(=O)N1CC2CC2(c2ncccn2)C1)C(F)(F)F ; C[C@H](Oc1ccc(S(C)(=O)=O)cc1C(=O)N1CC2CC2(c2noc(C3CC(F)(F)C3)n2)C1)C(F)(F)F ; C[C@H](Oc1ccc(S(C)(=O)=O)cc1C(=O)N1CC2CC2(c2nccc(C(F)(F)F)n2)C1)C(F)(F)F ; C[C@@H](Oc1ccc(S(C)(=O)=O)cc1C(=O)N1CC2CC2(c2nccc(C(F)(F)F)n2)C1)C(F)(F)F ; C[C@H](Oc1ccc(S(C)(=O)=O)cc1C(=O)N1CC2CC2(c2ncco2)C1)C(F)(F)F]

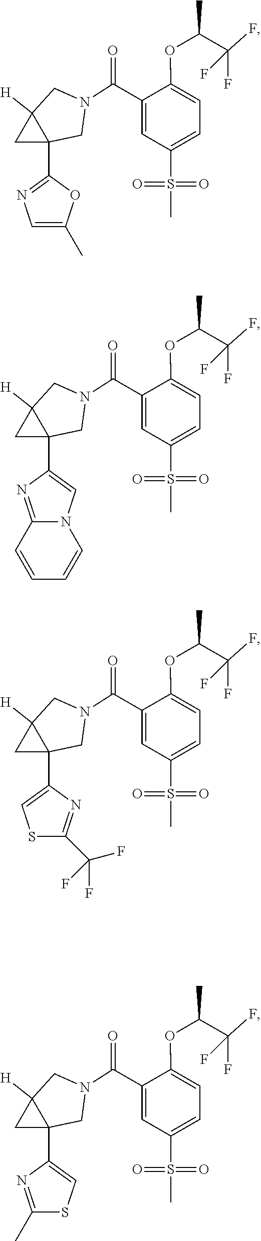 [SMILES: Cc1cnc(C23CC2CN(C(=O)c2cc(S(C)(=O)=O)ccc2O[C@@H](C)C(F)(F)F)C3)o1 ; C[C@H](Oc1ccc(S(C)(=O)=O)cc1C(=O)N1CC2CC2(c2cn3ccccc3n2)C1)C(F)(F)F ; C[C@H](Oc1ccc(S(C)(=O)=O)cc1C(=O)N1CC2CC2(c2csc(C(F)(F)F)n2)C1)C(F)(F)F ; Cc1nc(C23CC2CN(C(=O)c2cc(S(C)(=O)=O)ccc2O[C@@H](C)C(F)(F)F)C3)cs1]

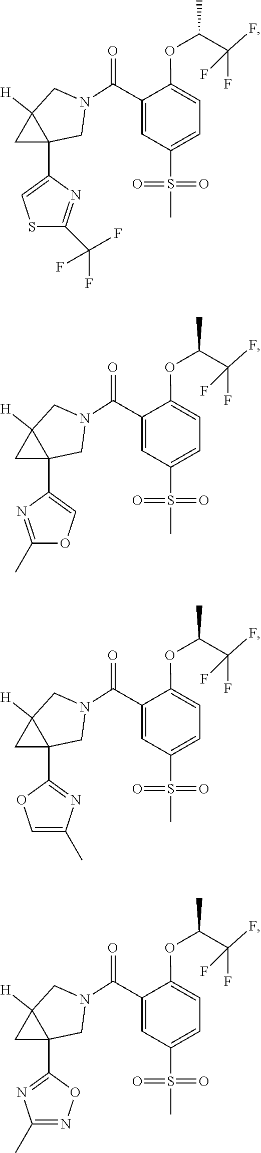 [SMILES: C[C@@H](Oc1ccc(S(C)(=O)=O)cc1C(=O)N1CC2CC2(c2csc(C(F)(F)F)n2)C1)C(F)(F)F ; Cc1nc(C23CC2CN(C(=O)c2cc(S(C)(=O)=O)ccc2O[C@@H](C)C(F)(F)F)C3)co1 ; Cc1coc(C23CC2CN(C(=O)c2cc(S(C)(=O)=O)ccc2O[C@@H](C)C(F)(F)F)C3)n1 ; Cc1noc(C23CC2CN(C(=O)c2cc(S(C)(=O)=O)ccc2O[C@@H](C)C(F)(F)F)C3)n1]

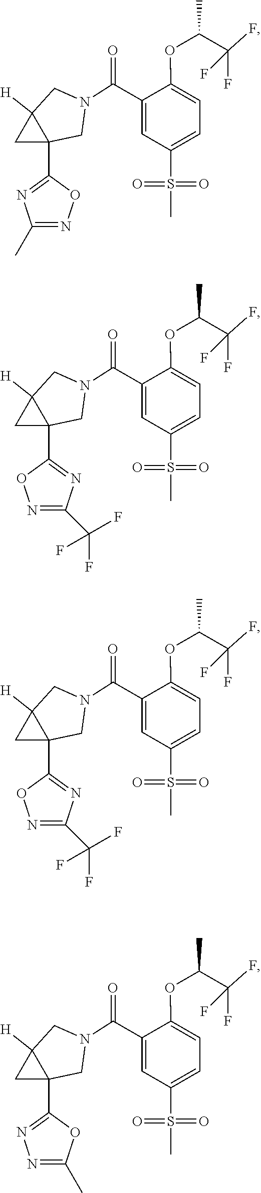 [SMILES: Cc1noc(C23CC2CN(C(=O)c2cc(S(C)(=O)=O)ccc2O[C@H](C)C(F)(F)F)C3)n1 ; C[C@H](Oc1ccc(S(C)(=O)=O)cc1C(=O)N1CC2CC2(c2nc(C(F)(F)F)no2)C1)C(F)(F)F ; C[C@@H](Oc1ccc(S(C)(=O)=O)cc1C(=O)N1CC2CC2(c2nc(C(F)(F)F)no2)C1)C(F)(F)F ; Cc1nnc(C23CC2CN(C(=O)c2cc(S(C)(=O)=O)ccc2O[C@@H](C)C(F)(F)F)C3)o1]

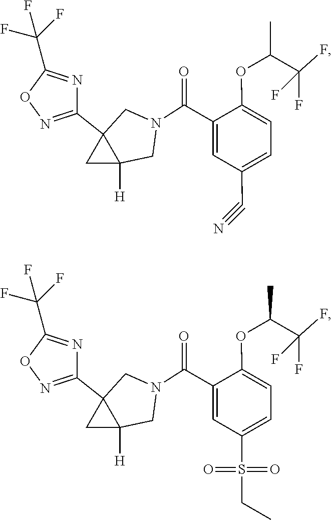 [SMILES: CC(Oc1ccc(C#N)cc1C(=O)N1CC2CC2(c2noc(C(F)(F)F)n2)C1)C(F)(F)F ; CCS(=O)(=O)c1ccc(O[C@@H](C)C(F)(F)F)c(C(=O)N2CC3CC3(c3noc(C(F)(F)F)n3)C2)c1]

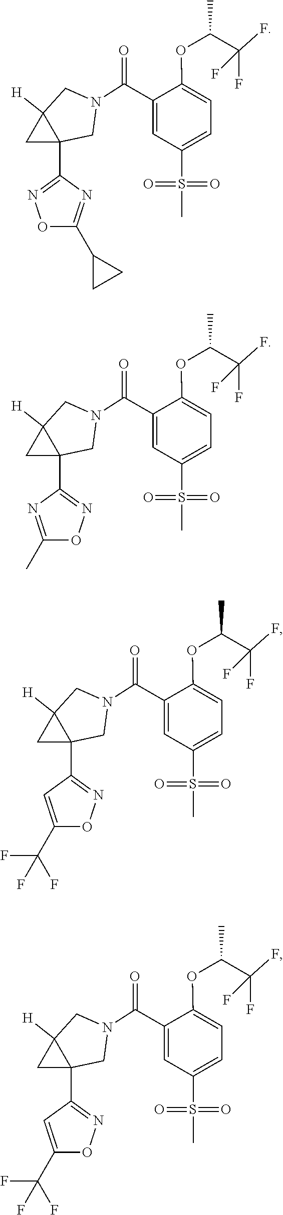 [SMILES: C[C@@H](Oc1ccc(S(C)(=O)=O)cc1C(=O)N1CC2CC2(c2noc(C3CC3)n2)C1)C(F)(F)F ; Cc1nc(C23CC2CN(C(=O)c2cc(S(C)(=O)=O)ccc2O[C@H](C)C(F)(F)F)C3)no1 ; C[C@H](Oc1ccc(S(C)(=O)=O)cc1C(=O)N1CC2CC2(c2cc(C(F)(F)F)on2)C1)C(F)(F)F ; C[C@@H](Oc1ccc(S(C)(=O)=O)cc1C(=O)N1CC2CC2(c2cc(C(F)(F)F)on2)C1)C(F)(F)F]

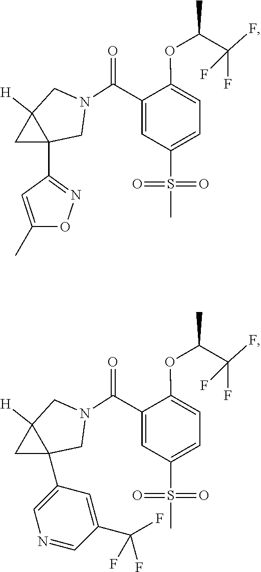 [SMILES: Cc1cc(C23CC2CN(C(=O)c2cc(S(C)(=O)=O)ccc2O[C@@H](C)C(F)(F)F)C3)no1 ; C[C@H](Oc1ccc(S(C)(=O)=O)cc1C(=O)N1CC2CC2(c2cncc(C(F)(F)F)c2)C1)C(F)(F)F]

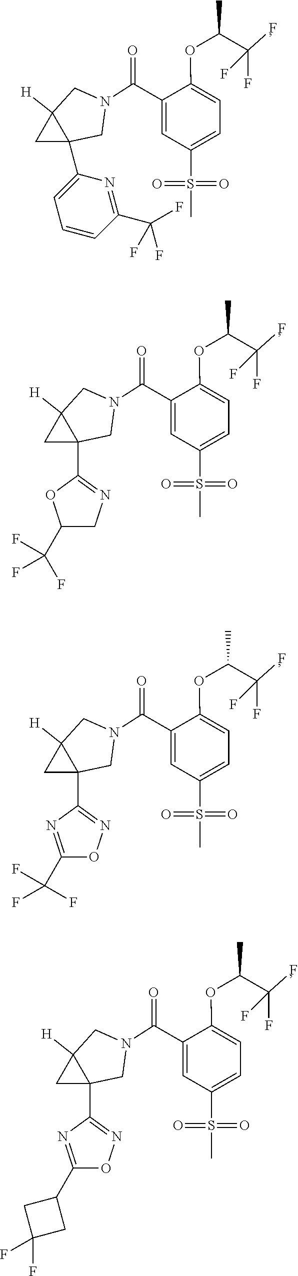 [SMILES: C[C@H](Oc1ccc(S(C)(=O)=O)cc1C(=O)N1CC2CC2(c2cccc(C(F)(F)F)n2)C1)C(F)(F)F ; C[C@H](Oc1ccc(S(C)(=O)=O)cc1C(=O)N1CC2CC2(C2=NCC(C(F)(F)F)O2)C1)C(F)(F)F ; C[C@@H](Oc1ccc(S(C)(=O)=O)cc1C(=O)N1CC2CC2(c2noc(C(F)(F)F)n2)C1)C(F)(F)F ; C[C@H](Oc1ccc(S(C)(=O)=O)cc1C(=O)N1CC2CC2(c2noc(C3CC(F)(F)C3)n2)C1)C(F)(F)F]

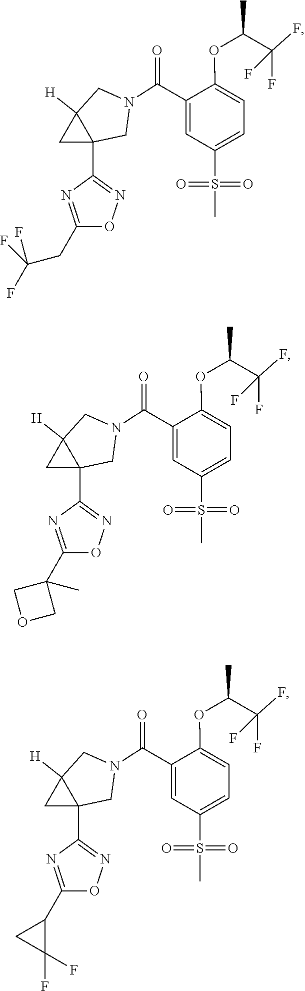 [SMILES: C[C@H](Oc1ccc(S(C)(=O)=O)cc1C(=O)N1CC2CC2(c2noc(CC(F)(F)F)n2)C1)C(F)(F)F ; C[C@H](Oc1ccc(S(C)(=O)=O)cc1C(=O)N1CC2CC2(c2noc(C3(C)COC3)n2)C1)C(F)(F)F ; C[C@H](Oc1ccc(S(C)(=O)=O)cc1C(=O)N1CC2CC2(c2noc(C3CC3(F)F)n2)C1)C(F)(F)F]

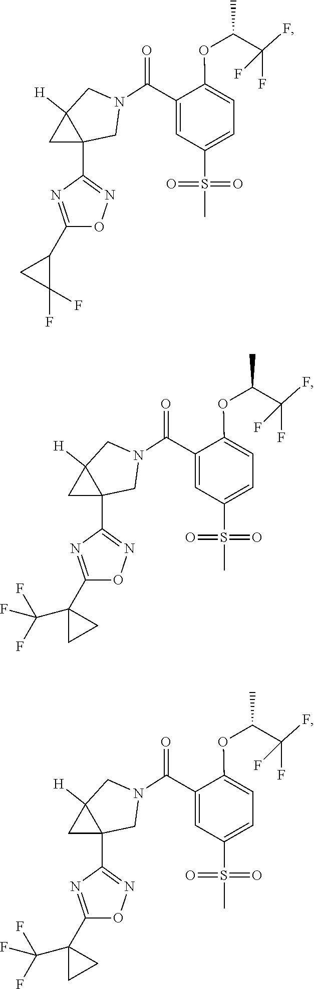 [SMILES: C[C@@H](Oc1ccc(S(C)(=O)=O)cc1C(=O)N1CC2CC2(c2noc(C3CC3(F)F)n2)C1)C(F)(F)F ; C[C@H](Oc1ccc(S(C)(=O)=O)cc1C(=O)N1CC2CC2(c2noc(C3(C(F)(F)F)CC3)n2)C1)C(F)(F)F ; C[C@@H](Oc1ccc(S(C)(=O)=O)cc1C(=O)N1CC2CC2(c2noc(C3(C(F)(F)F)CC3)n2)C1)C(F)(F)F]

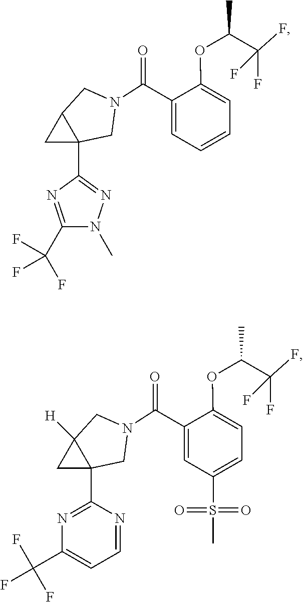 [SMILES: C[C@H](Oc1ccccc1C(=O)N1CC2CC2(c2nc(C(F)(F)F)n(C)n2)C1)C(F)(F)F ; C[C@@H](Oc1ccc(S(C)(=O)=O)cc1C(=O)N1CC2CC2(c2nccc(C(F)(F)F)n2)C1)C(F)(F)F]

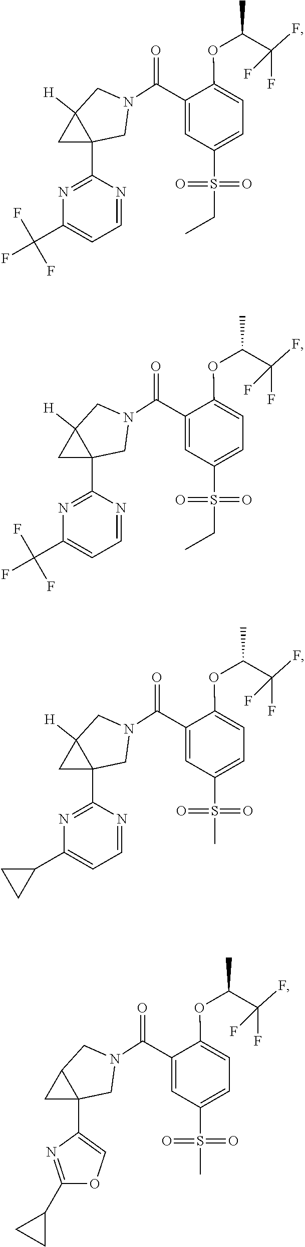 [SMILES: CCS(=O)(=O)c1ccc(O[C@@H](C)C(F)(F)F)c(C(=O)N2CC3CC3(c3nccc(C(F)(F)F)n3)C2)c1 ; CCS(=O)(=O)c1ccc(O[C@H](C)C(F)(F)F)c(C(=O)N2CC3CC3(c3nccc(C(F)(F)F)n3)C2)c1 ; C[C@@H](Oc1ccc(S(C)(=O)=O)cc1C(=O)N1CC2CC2(c2nccc(C3CC3)n2)C1)C(F)(F)F ; C[C@H](Oc1ccc(S(C)(=O)=O)cc1C(=O)N1CC2CC2(c2coc(C3CC3)n2)C1)C(F)(F)F]

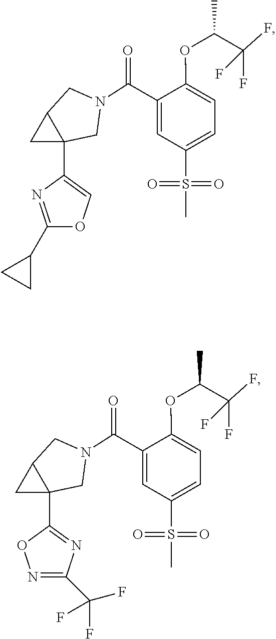 [SMILES: C[C@@H](Oc1ccc(S(C)(=O)=O)cc1C(=O)N1CC2CC2(c2coc(C3CC3)n2)C1)C(F)(F)F ; C[C@H](Oc1ccc(S(C)(=O)=O)cc1C(=O)N1CC2CC2(c2nc(C(F)(F)F)no2)C1)C(F)(F)F]

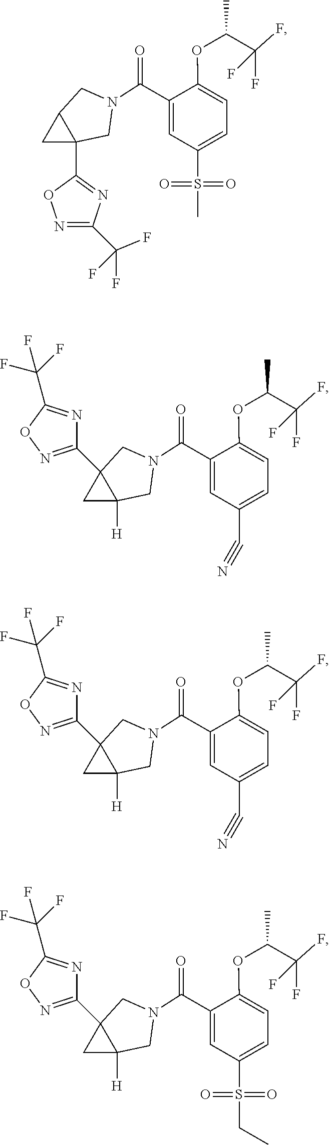 [SMILES: C[C@@H](Oc1ccc(S(C)(=O)=O)cc1C(=O)N1CC2CC2(c2nc(C(F)(F)F)no2)C1)C(F)(F)F ; C[C@H](Oc1ccc(C#N)cc1C(=O)N1CC2CC2(c2noc(C(F)(F)F)n2)C1)C(F)(F)F ; C[C@@H](Oc1ccc(C#N)cc1C(=O)N1CC2CC2(c2noc(C(F)(F)F)n2)C1)C(F)(F)F ; CCS(=O)(=O)c1ccc(O[C@H](C)C(F)(F)F)c(C(=O)N2CC3CC3(c3noc(C(F)(F)F)n3)C2)c1]

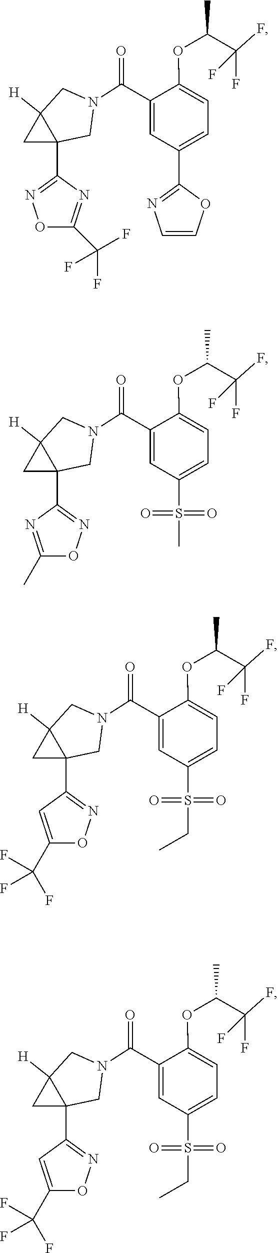 [SMILES: C[C@H](Oc1ccc(-c2ncco2)cc1C(=O)N1CC2CC2(c2noc(C(F)(F)F)n2)C1)C(F)(F)F ; Cc1nc(C23CC2CN(C(=O)c2cc(S(C)(=O)=O)ccc2O[C@H](C)C(F)(F)F)C3)no1 ; CCS(=O)(=O)c1ccc(O[C@@H](C)C(F)(F)F)c(C(=O)N2CC3CC3(c3cc(C(F)(F)F)on3)C2)c1 ; CCS(=O)(=O)c1ccc(O[C@H](C)C(F)(F)F)c(C(=O)N2CC3CC3(c3cc(C(F)(F)F)on3)C2)c1]

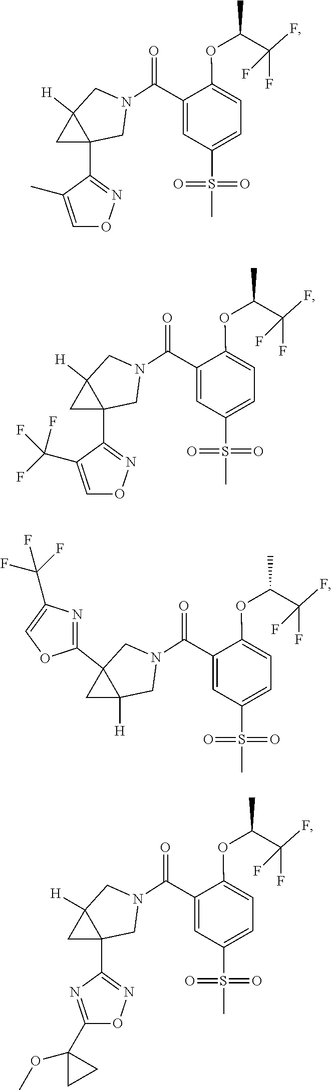

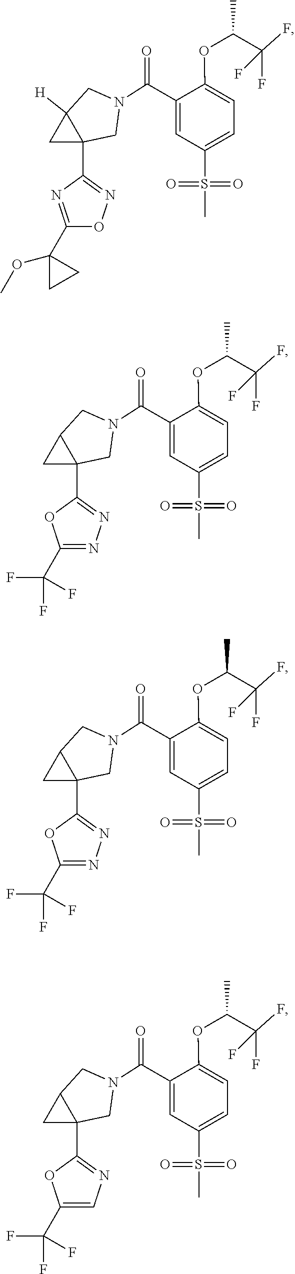 [SMILES: COC1(c2nc(C34CC3CN(C(=O)c3cc(S(C)(=O)=O)ccc3O[C@H](C)C(F)(F)F)C4)no2)CC1 ; C[C@@H](Oc1ccc(S(C)(=O)=O)cc1C(=O)N1CC2CC2(c2nnc(C(F)(F)F)o2)C1)C(F)(F)F ; C[C@H](Oc1ccc(S(C)(=O)=O)cc1C(=O)N1CC2CC2(c2nnc(C(F)(F)F)o2)C1)C(F)(F)F ; C[C@@H](Oc1ccc(S(C)(=O)=O)cc1C(=O)N1CC2CC2(c2ncc(C(F)(F)F)o2)C1)C(F)(F)F]

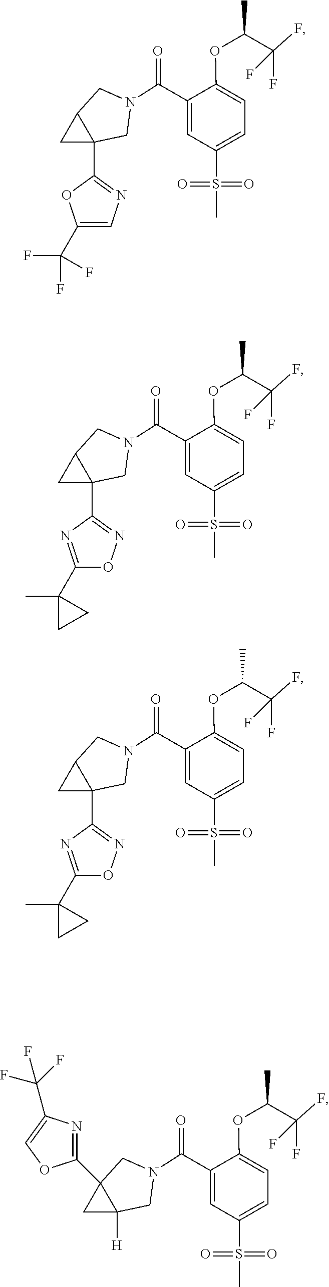 [SMILES: C[C@H](Oc1ccc(S(C)(=O)=O)cc1C(=O)N1CC2CC2(c2ncc(C(F)(F)F)o2)C1)C(F)(F)F ; C[C@H](Oc1ccc(S(C)(=O)=O)cc1C(=O)N1CC2CC2(c2noc(C3(C)CC3)n2)C1)C(F)(F)F ; C[C@@H](Oc1ccc(S(C)(=O)=O)cc1C(=O)N1CC2CC2(c2noc(C3(C)CC3)n2)C1)C(F)(F)F ; C[C@H](Oc1ccc(S(C)(=O)=O)cc1C(=O)N1CC2CC2(c2nc(C(F)(F)F)co2)C1)C(F)(F)F]

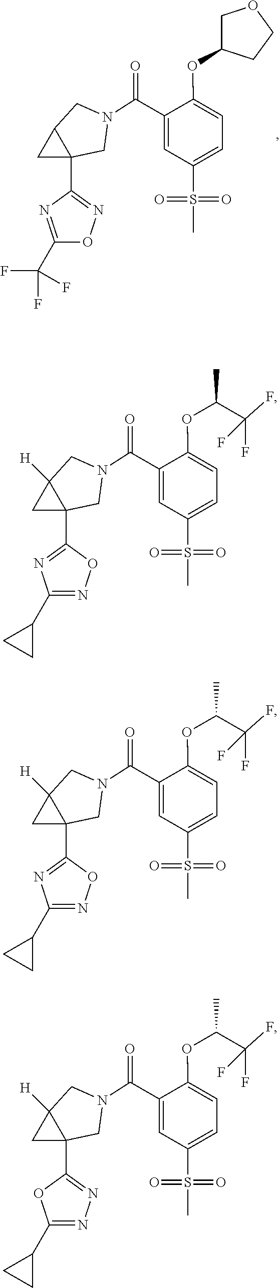 [SMILES: CS(=O)(=O)c1ccc(O[C@@H]2CCOC2)c(C(=O)N2CC3CC3(c3noc(C(F)(F)F)n3)C2)c1 ; C[C@H](Oc1ccc(S(C)(=O)=O)cc1C(=O)N1CC2CC2(c2nc(C3CC3)no2)C1)C(F)(F)F ; C[C@@H](Oc1ccc(S(C)(=O)=O)cc1C(=O)N1CC2CC2(c2nc(C3CC3)no2)C1)C(F)(F)F ; C[C@@H](Oc1ccc(S(C)(=O)=O)cc1C(=O)N1CC2CC2(c2nnc(C3CC3)o2)C1)C(F)(F)F]

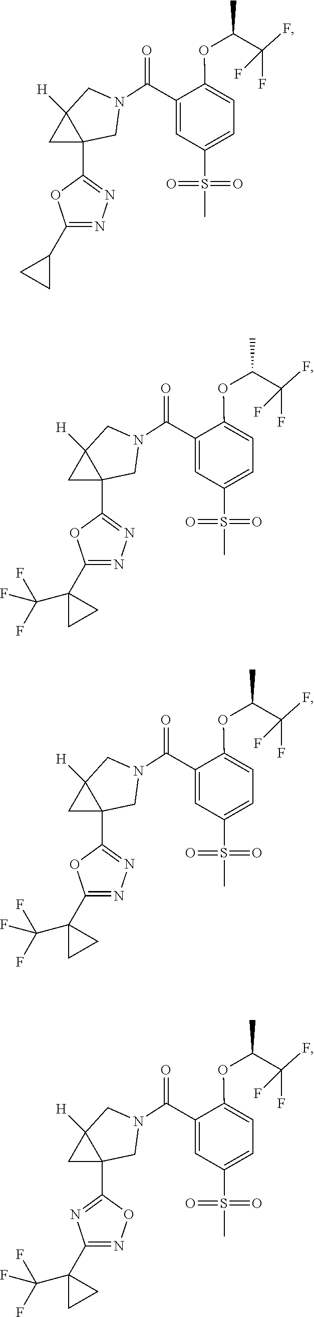 [SMILES: C[C@H](Oc1ccc(S(C)(=O)=O)cc1C(=O)N1CC2CC2(c2nnc(C3CC3)o2)C1)C(F)(F)F ; C[C@@H](Oc1ccc(S(C)(=O)=O)cc1C(=O)N1CC2CC2(c2nnc(C3(C(F)(F)F)CC3)o2)C1)C(F)(F)F ; C[C@H](Oc1ccc(S(C)(=O)=O)cc1C(=O)N1CC2CC2(c2nnc(C3(C(F)(F)F)CC3)o2)C1)C(F)(F)F ; C[C@H](Oc1ccc(S(C)(=O)=O)cc1C(=O)N1CC2CC2(c2nc(C3(C(F)(F)F)CC3)no2)C1)C(F)(F)F]

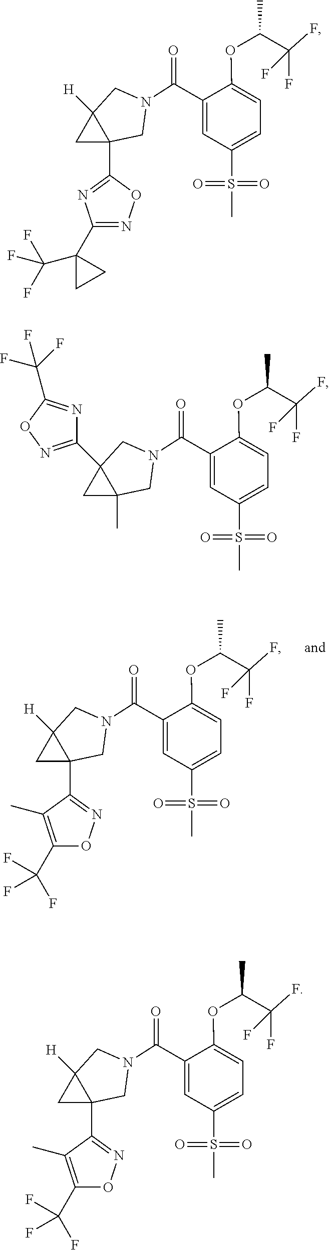

In certain embodiments, the compound of Formula X is a compound having a formula

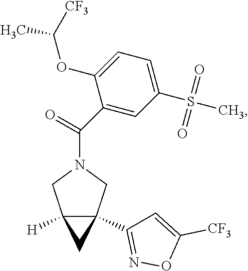 [SMILES: C[C@@H](Oc1ccc(S(C)(=O)=O)cc1C(=O)N1C[C@@H]2C[C@]2(c2cc(C(F)(F)F)on2)C1)C(F)(F)F]

or a pharmaceutically acceptable salt thereof.

In some embodiments of the methods and uses disclosed herein, the GlyT1 inhibitor is a compound of Formula XI,

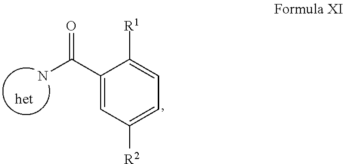

wherein:
R¹ is halogen, —OR^{1′}, SR^{1″}, cycloalkyl, cyclic amide, heterocycloalkyl, aryl or 5- or 6-membered heteroaryl containing one, two or three heteroatoms selected from the group consisting of oxygen, sulphur and nitrogen;
R^{1′} and R^{1″} are each independently hydrogen, lower alkyl, lower alkyl substituted by halogen, —(CH₂)ₓ-cycloalkyl or —(CH₂)ₓ-aryl;
R² is —S(O)₂-lower alkyl, —S(O)₂NH-lower alkyl, NO₂ or CN;

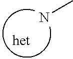

is an aromatic or partially aromatic bicyclic amine, having one or two additional N-atoms selected from the group consisting of

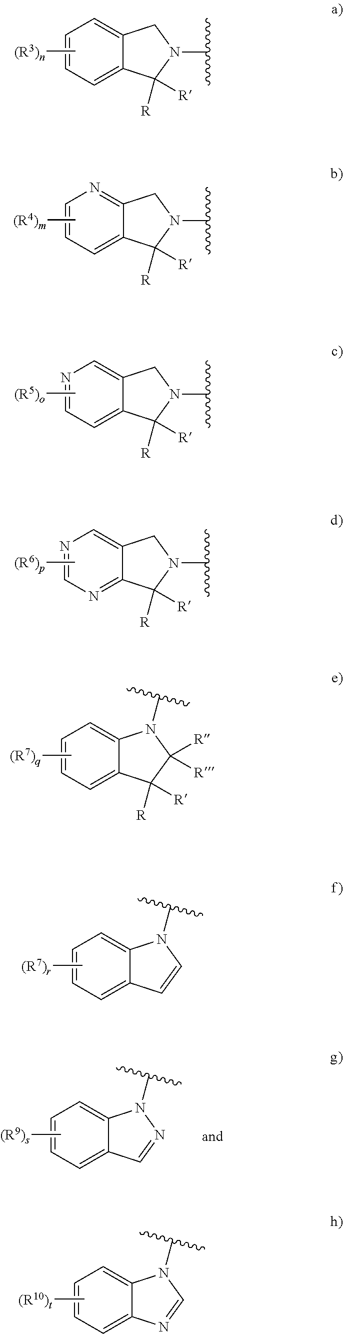

and wherein one of the additional N-ring atoms of the aromatic or partially aromatic bicyclic amine can be available in form of its oxide

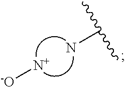

R3 to R10 are each independently hydrogen, hydroxy, halogen, ═O, lower alkyl, cycloalkyl, heterocycloalkyl, lower alkoxy, CN, NO2, NH2, aryl, 5- or 6-membered heteroaryl containing one, two or three heteroatoms selected from the group consisting of oxygen, sulphur and nitrogen, NH-lower alkyl, N(lower alkyl)2, cyclic amide, —C(O)-cyclic amide, S-lower alkyl, S(O)2-lower alkyl, lower alkyl substituted by halogen, lower alkoxy substituted by halogen, lower alkyl substituted by hydroxy, —O—(CH2)y-lower alkoxy, —O(CH2)yC(O)N(lower alkyl)2, —C(O)-lower alkyl, —O—(CH2)x-aryl, —O—(CH2)x-cycloalkyl, —O—(CH2)x-heterocycloalkyl, —C(O)O-lower alkyl, —C(O)—NH-lower alkyl, —C(O)—N(lower alkyl)2, 2-oxy-5-aza-bicyclo[2.2.1]hept-5-yl or 3-oxa-8-aza-bicyclo[3.2.1]oct-8-yl;
R, R′, R″ and R′″ are each independently hydrogen or lower alkyl; or
R′ and R′″ in group e) together with —(CH2)4- form a six membered ring;
and wherein all aryl-, cycloalkyl-, cyclic amide, heterocycloalkyl- or 5 or 6 membered heteroaryl groups as defined for R1, R1′, R1″ and R3 to R10 are unsubstituted or substituted by one or more substituents selected from the group consisting of hydroxy, ═O, halogen, lower allyl, phenyl, lower alkyl substituted by halogen and lower alkoxy;
n, m, o, p, q, r, s and t are each independently 1 or 2;
x is 0, 1 or 2; and
y is 1 or 2;
or a pharmaceutically acceptable acid addition salt thereof.

In certain embodiments, the compound of formula XI, or a pharmaceutically acceptable salt thereof, is a compound of formula XI(a),

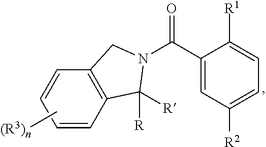

or a pharmaceutically acceptable salt thereof, a compound of formula XI(b),

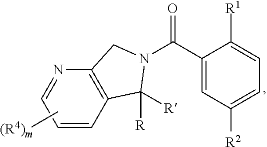

or a pharmaceutically acceptable salt thereof, a compound of formula XI(c),

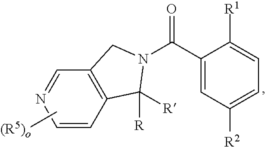

or a pharmaceutically acceptable salt thereof, a compound of formula XI(d),

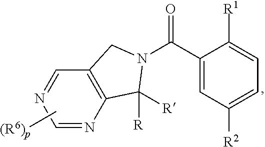

or a pharmaceutically acceptable salt thereof, a compound of formula XI(e),

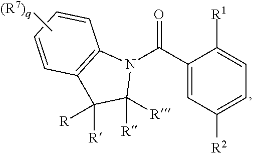

or a pharmaceutically acceptable salt thereof, a compound of formula XI(f),

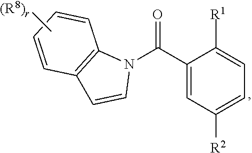

or a pharmaceutically acceptable salt thereof, a compound of formula XI(g),

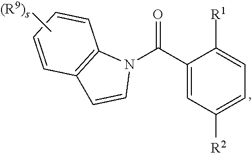

or a pharmaceutically acceptable salt thereof, or a compound of formula XI(h),

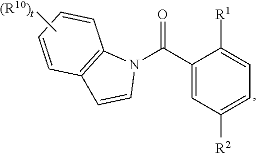

or a pharmaceutically acceptable salt thereof.

In certain embodiments, the compound of formula XI is a compound selected from any of the following, a stereoisomer or stereoisomeric mixture thereof, or a pharmaceutically acceptable salt thereof:

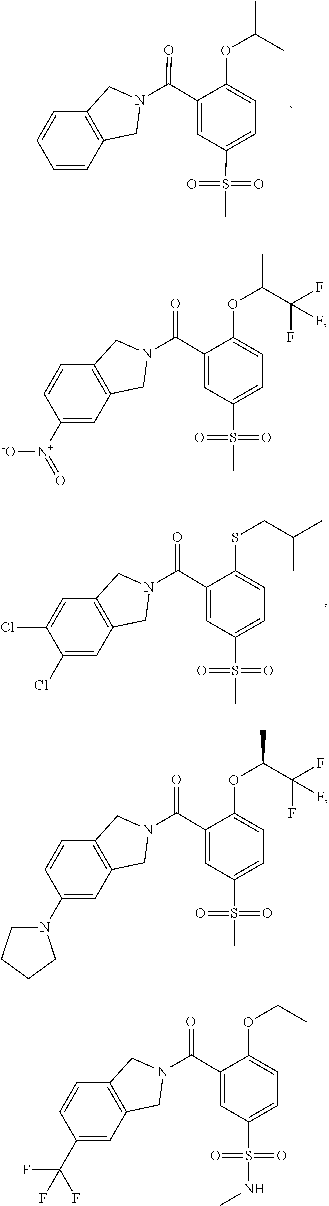

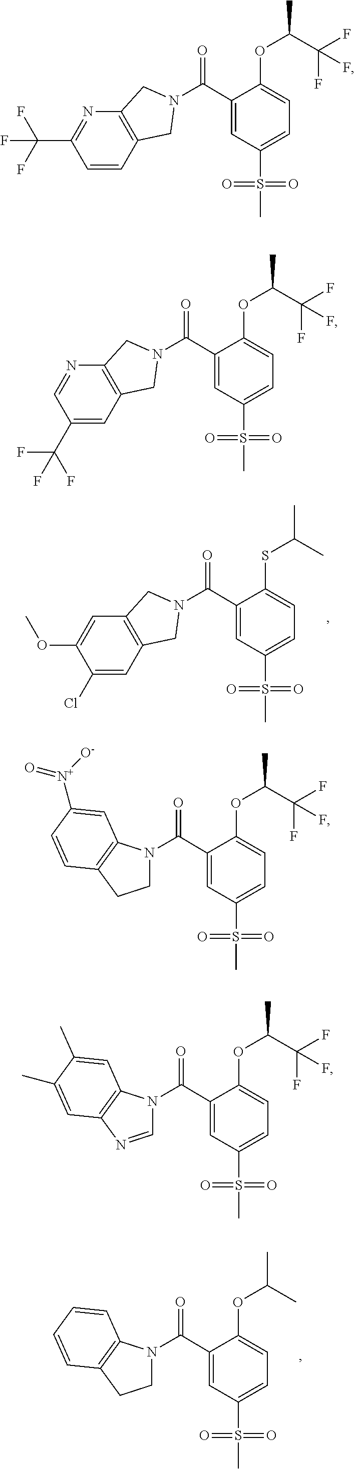

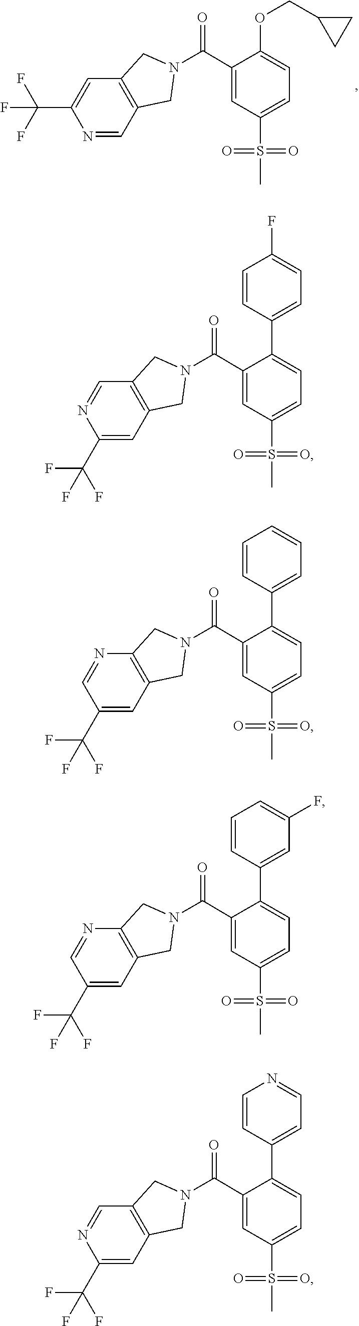

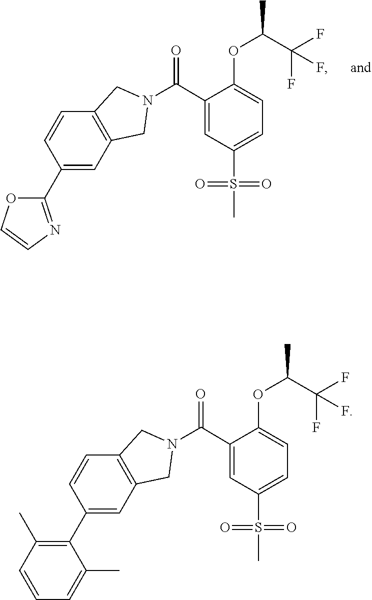 [SMILES: C[C@H](Oc1ccc(S(C)(=O)=O)cc1C(=O)N1Cc2ccc(-c3ncco3)cc2C1)C(F)(F)F ; Cc1cccc(C)c1-c1ccc2c(c1)CN(C(=O)c1cc(S(C)(=O)=O)ccc1O[C@@H](C)C(F)(F)F)C2]

In certain of the methods and uses disclosed herein, the subject is a subject in need thereof.

In some embodiments of the uses and methods as disclosed herein, the glycine transporter inhibitor, such as a GlyT1 inhibitor (e.g., a GlyT1 inhibitor as disclosed herein), or a pharmaceutically acceptable salt thereof, or a prodrug of the glycine transporter inhibitor, such as a GlyT1 inhibitor (e.g., a GlyT1 inhibitor as disclosed herein), or its pharmaceutically acceptable salt is administered in a therapeutically effective amount.

In some embodiments, a compound, or a pharmaceutically acceptable salt, solvate or prodrug thereof, is chosen from a compound of as described herein. Any of the compounds provided for herein can be prepared as pharmaceutically acceptable salts, solvates or prodrugs and/or as part of a pharmaceutical composition as descripted in the cited patents or patent application publications herein.

Although the compounds described herein may be shown with specific stereochemistries around certain atoms, such as cis or trans, the compounds can also be made in the opposite orientation or in a racemic mixture. Such isomers or racemic mixtures are encompassed by the present disclosure. Additionally, although the compounds are shown collectively in a table, any compounds, or a pharmaceutically acceptable salt, solvate or prodrug thereof, can be chosen from the table and used in the embodiments provided for herein.

The compounds described herein can be made according to the methods described in the cited patents or patent application publications herein.

The compounds can be used to inhibit the GlyT1 transporter. Thus, in some embodiments, the compounds can be referred to as GlyT1 transporter inhibiting compounds or GlyT1 inhibitors.

The compounds described herein can be administered in any conventional manner by any route where they are active. Administration can be systemic, topical, or oral. For example, administration can be, but is not limited to, parenteral, subcutaneous, intravenous, intramuscular, intraperitoneal, transdermal, oral, buccal, sublingual, or ocular routes, or intravaginal, by inhalation, by depot injections, or by implants. The mode of administration can depend on the conditions or disease to be targeted or treated. The selection of the specific route of administration can be selected or adjusted by the clinician according to methods known to the clinician to obtain the desired clinical response.

In some embodiments, it may be desirable to administer one or more compounds, or a pharmaceutically acceptable salt, solvate or prodrug thereof, locally to an area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, wherein the implant is of a porous, non-porous, or gelatinous material, including membranes, such as silastic membranes, or fibers.

The compounds described herein can be administered either alone or in combination (concurrently or serially) with other pharmaceuticals. For example, the compounds can be administered in combination with other drugs for the treatment of anemia associated with a ribosomal disorder and the like. Examples of other pharmaceuticals or medicaments are known to one of skill in the art and include, but are not limited to those described herein.

The means and methods for administration are known in the art and an artisan can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician). The standard dosing for protamine can be used and adjusted (i.e., increased or decreased) depending upon the factors described above. The selection of the specific dose regimen can be selected or adjusted or titrated by the clinician according to methods known to the clinician to obtain the desired clinical response.

The amount of a compound described herein that will be effective in the treatment and/or prevention of a particular disease, condition, or disorder will depend on the nature and extent of the disease, condition, or disorder, and can be determined by standard clinical techniques. In addition, in vitro or in vivo assays may optionally be employed to help identify optimal dosage ranges. The precise dose to be employed in the compositions will also depend on the route of administration, and the seriousness of the disorder, and should be decided according to the judgment of the practitioner and each patient's circumstances. However, a suitable dosage range for oral administration is, generally, from about 0.001 milligram to about 200 milligrams per kilogram body weight, from about 0.01 milligram to about 100 milligrams per kilogram body weight, from about 0.01 milligram to about 70 milligrams per kilogram body weight, from about 0.1 milligram to about 50 milligrams per kilogram body weight, from 0.5 milligram to about 20 milligrams per kilogram body weight, or from about 1 milligram to about 10 milligrams per kilogram body weight. In some embodiments, the oral dose is about 5 milligrams per kilogram body weight.

In some embodiments, suitable dosage ranges for intravenous (i.v.) administration are from about 0.01 mg to about 500 mg per kg body weight, from about 0.1 mg to about 100 mg per kg body weight, from about 1 mg to about 50 mg per kg body weight, or from about mg to about 35 mg per kg body weight. Suitable dosage ranges for other modes of administration can be calculated based on the forgoing dosages as known by those skilled in the art. For example, recommended dosages for intranasal, transmucosal, intradermal, intramuscular, intraperitoneal, subcutaneous, epidural, sublingual, intracerebral, intravaginal, transdermal administration or administration by inhalation are in the range of from about 0.001 mg to about 200 mg per kg of body weight, from about 0.01 mg to about 100 mg per kg of body weight, from about 0.1 mg to about 50 mg per kg of body weight, or from about 1 mg to about 20 mg per kg of body weight. Effective doses may be extrapolated from dose-response curves derived from in vitro or animal model test systems. Such animal models and systems are well known in the art.

In certain embodiments, the glycine transporter inhibitor to be administered is a GlyT1 inhibitor, such as a GlyT1 inhibitor as disclosed herein. In some embodiments, suitable dosage ranges for the GlyT1 inhibitor are from about 5 mg/day to 200 mg/day. In some embodiments, the GlyT1 inhibitor is administered at 5 mg/day. In some embodiments, the GlyT1 inhibitor is administered at 10 mg/day. In some embodiments, the GlyT1 inhibitor is administered at 15 mg/day. In some embodiments, the GlyT1 inhibitor is administered at 20 mg/day. In some embodiments, the GlyT1 inhibitor is administered at 25 mg/day. In some embodiments, the GlyT1 inhibitor is administered at 30 mg/day. In some embodiments, the GlyT1 inhibitor is administered at 35 mg/day. In some embodiments, the GlyT1 inhibitor is administered at 40 mg/day. In some embodiments, the GlyT1 inhibitor is administered at 45 mg/day. In some embodiments, the GlyT1 inhibitor is administered at 50 mg/day. In some embodiments, the GlyT1 inhibitor is administered at 55 mg/day. In some embodiments, the GlyT1 inhibitor is administered at 60 mg/day. In some embodiments, the GlyT1 inhibitor is administered at 65 mg/day. In some embodiments, the GlyT1 inhibitor is administered at 70 mg/day. In some embodiments, the GlyT1 inhibitor is administered at 75 mg/day. In some embodiments, the GlyT1 inhibitor is administered at 80 mg/day. In some embodiments, the GlyT1 inhibitor is administered at 85 mg/day. In some embodiments, the GlyT1 inhibitor is administered at 90 mg/day. In some embodiments, the GlyT1 inhibitor is administered at 95 mg/day. In some embodiments, the GlyT1 inhibitor is administered at 100 mg/day. In some embodiments, the GlyT1 inhibitor is administered at 105 mg/day. In some embodiments, the GlyT1 inhibitor is administered at 110 mg/day. In some embodiments, the GlyT1 inhibitor is administered at 115 mg/day. In some embodiments, the GlyT1 inhibitor is administered at 120 mg/day. In some embodiments, the GlyT1 inhibitor is administered at 125 mg/day. In some embodiments, the GlyT1 inhibitor is administered at 130 mg/day. In some embodiments, the GlyT1 inhibitor is administered at 135 mg/day. In some embodiments, the GlyT1 inhibitor is administered at 140 mg/day. In some embodiments, the GlyT1 inhibitor is administered at 145 mg/day. In some embodiments, the GlyT1 inhibitor is administered at 150 mg/day. In some embodiments, the GlyT1 inhibitor is administered at 155 mg/day. In some embodiments, the GlyT1 inhibitor is administered at 160 mg/day. In some embodiments, the GlyT1 inhibitor is administered at 165 mg/day. In some embodiments, the GlyT1 inhibitor is administered at 170 mg/day. In some embodiments, the GlyT1 inhibitor is administered at 175 mg/day. In some embodiments, the GlyT1 inhibitor is administered at 180 mg/day. In some embodiments, the GlyT1 inhibitor is administered at 185 mg/day. In some embodiments, the GlyT1 inhibitor is administered at 190 mg/day. In some embodiments, the GlyT1 inhibitor is administered at 195 mg/day. In some embodiments, the GlyT1 inhibitor is administered at 200 mg/day.

In certain embodiments, the glycine transporter inhibitor to be administered is a GlyT1 inhibitor, such as bitopertin, pharmaceutically acceptable salt thereof, or a prodrug of bitopertin or its pharmaceutically acceptable salt. In some embodiments, the GlyT1 inhibitor is bitopertin. In some embodiments, suitable dosage ranges for bitopertin are from about 5 mg/day to 200 mg/day. In some embodiments, bitopertin is administered at 5 mg/day. In some embodiments, bitopertin is administered at 10 mg/day. In some embodiments, bitopertin is administered at 15 mg/day. In some embodiments, bitopertin is administered at 20 mg/day. In some embodiments, bitopertin is administered at 25 mg/day. In some embodiments, bitopertin is administered at 30 mg/day. In some embodiments, bitopertin is administered at 35 mg/day. In some embodiments, bitopertin is administered at 40 mg/day. In some embodiments, bitopertin is administered at 45 mg/day. In some embodiments, bitopertin is administered at 50 mg/day. In some embodiments, bitopertin is administered at 55 mg/day. In some embodiments, bitopertin is administered at 60 mg/day. In some embodiments, bitopertin is administered at 65 mg/day. In some embodiments, bitopertin is administered at 70 mg/day. In some embodiments, bitopertin is administered at 75 mg/day. In some embodiments, bitopertin is administered at 80 mg/day. In some embodiments, bitopertin is administered at 85 mg/day. In some embodiments, bitopertin is administered at 90 mg/day. In some embodiments, bitopertin is administered at 95 mg/day. In some embodiments, bitopertin is administered at 100 mg/day. In some embodiments, bitopertin is administered at 105 mg/day. In some embodiments, bitopertin is administered at 110 mg/day. In some embodiments, bitopertin is administered at 115 mg/day. In some embodiments, bitopertin is administered at 120 mg/day. In some embodiments, bitopertin is administered at 125 mg/day. In some embodiments, bitopertin is administered at 130 mg/day. In some embodiments, bitopertin is administered at 135 mg/day. In some embodiments, bitopertin is administered at 140 mg/day. In some embodiments, bitopertin is administered at 145 mg/day. In some embodiments, bitopertin is administered at 150 mg/day. In some embodiments, bitopertin is administered at 155 mg/day. In some embodiments, bitopertin is administered at 160 mg/day. In some embodiments, bitopertin is administered at 165 mg/day. In some embodiments, bitopertin is administered at 170 mg/day. In some embodiments, bitopertin is administered at 175 mg/day. In some embodiments, bitopertin is administered at 180 mg/day. In some embodiments, bitopertin is administered at 185 mg/day. In some embodiments, bitopertin is administered at 190 mg/day. In some embodiments, bitopertin is administered at 195 mg/day. In some embodiments, bitopertin is administered at 200 mg/day.

The compounds described herein can be formulated for parenteral administration by injection, such as by bolus injection or continuous infusion. In some embodiments, the compounds can be administered by continuous infusion subcutaneously over a period of about 15 minutes to about 24 hours. Formulations for injection can be presented in unit dosage form, such as in ampoules or in multi-dose containers, with an optionally added preservative. The compositions can take such forms as suspensions, solutions or emulsions in oily or aqueous vehicles, and can contain formulatory agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a solution, suspension, emulsion, or dry powder.

For oral administration, the compounds described herein can be formulated by combining the compounds with pharmaceutically acceptable carriers well known in the art. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as mannitol, lactose, starch, magnesium stearate, sodium saccharine, cellulose, magnesium carbonate, etc. Such vehicles are suitably of pharmaceutical grade.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

For buccal administration, the compositions can take the form of, such as, tablets or lozenges formulated in a conventional manner.

For administration by inhalation, the compounds described herein can be delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

The compounds described herein can also be formulated in rectal compositions such as suppositories or retention enemas, such as containing conventional suppository bases such as cocoa butter or other glycerides. The compounds described herein can also be formulated in vaginal compositions such as vaginal creams, suppositories, pessaries, vaginal rings, and intrauterine devices.

In transdermal administration, the compounds can be applied to a plaster, or can be applied by transdermal, therapeutic systems that are consequently supplied to the organism. In some embodiments, the compounds are present in creams, solutions, powders, fluid emulsions, fluid suspensions, semi-solids, ointments, pastes, gels, jellies, and foams, or in patches containing any of the same.

The compounds described herein can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In some embodiments, the compounds can be delivered in a controlled release system. In one embodiment, a pump may be used (see Langer, supra; Sefton, CRC Crit. Ref. Biomed. Eng., 1987, 14, 201; Buchwald et al., Surgery, 1980, 88, 507 Saudek et al., N. Engl. J. Med., 1989, 321, 574). In some embodiments, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger et al., J. Macromol. Sci. Rev. Macromol. Chem., 1983, 23, 61; see, also Levy et al., Science, 1985, 228, 190; During et al., Ann. Neurol., 1989, 25, 351; Howard et al., J. Neurosurg., 1989, 71, 105). In yet another embodiment, a controlled-release system can be placed in proximity of the target of the compounds described herein, such as the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, Science, 1990, 249, 1527-1533) may be used.

It is also known in the art that the compounds can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol).

In some embodiments, the compounds described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

Suitable compositions include, but are not limited to, oral non-absorbed compositions. Suitable compositions also include, but are not limited to saline, water, cyclodextrin solutions, and buffered solutions of pH 3-9.

The compounds described herein, or pharmaceutically acceptable salts, solvates or prodrugs thereof, can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, and 1.2% saline, and any combination thereof. In some embodiments, excipient is chosen from propylene glycol, purified water, and glycerin.

In some embodiments, the formulation can be lyophilized to a solid and reconstituted with, for example, water prior to use.

When administered to a mammal (e.g., to an animal for veterinary use or to a human for clinical use) the compounds can be administered in isolated form.

When administered to a human, the compounds can be sterile. Water is a suitable carrier when the compound of Formula I-VIII is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions described herein can take the form of a solution, suspension, emulsion, tablet, pill, pellet, capsule, capsule containing a liquid, powder, sustained-release formulation, suppository, aerosol, spray, or any other form suitable for use. Examples of suitable pharmaceutical carriers are described in Remington's Pharmaceutical Sciences, A.R. Gennaro (Editor) Mack Publishing Co.

In some embodiments, the compounds are formulated in accordance with routine procedures as a pharmaceutical composition adapted for administration to humans. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

In some embodiments, a composition is in the form of a liquid wherein the active agent (i.e., one of the facially amphiphilic polymers or oligomers disclosed herein) is present in solution, in suspension, as an emulsion, or as a solution/suspension. In some embodiments, the liquid composition is in the form of a gel. In other embodiments, the liquid composition is aqueous. In other embodiments, the composition is in the form of an ointment.

In some embodiments, the composition is in the form of a solid article. For example, in some embodiments, the ophthalmic composition is a solid article that can be inserted in a suitable location in the eye, such as between the eye and eyelid or in the conjunctival sac, where it releases the active agent as described, for example, U.S. Pat. Nos. 3,863,633; 3,867,519; 3,868,445; 3,960,150; 3,963,025; 4,186,184; 4,303,637; 5,443,505; and 5,869,079. Release from such an article is usually to the cornea, either via the lacrimal fluid that bathes the surface of the cornea, or directly to the cornea itself, with which the solid article is generally in intimate contact. Solid articles suitable for implantation in the eye in such fashion are generally composed primarily of polymers and can be bioerodible or non-bioerodible. Bioerodible polymers that can be used in the preparation of ocular implants carrying one or more of compounds include, but are not limited to, aliphatic polyesters such as polymers and copolymers of poly(glycolide), poly(lactide), poly(epsilon-caprolactone), poly-(hydroxybutyrate) and poly(hydroxyvalerate), polyamino acids, polyorthoesters, polyanhydrides, aliphatic polycarbonates and polyether lactones. Suitable non-bioerodible polymers include silicone elastomers.

The compositions described herein can contain preservatives. Suitable preservatives include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof, phenoxyethanol; chlorophenoxyethanol; phenoxypropanol; chlorobutanol; chlorocresol; phenylethyl alcohol; disodium EDTA; and sorbic acid and salts thereof.

Optionally one or more stabilizers can be included in the compositions to enhance chemical stability where required. Suitable stabilizers include, but are not limited to, chelating agents or complexing agents, such as, for example, the calcium complexing agent ethylene diamine tetraacetic acid (EDTA). For example, an appropriate amount of EDTA or a salt thereof, e.g., the disodium salt, can be included in the composition to complex excess calcium ions and prevent gel formation during storage. EDTA or a salt thereof can suitably be included in an amount of about 0.01% to about 0.5%. In those embodiments containing a preservative other than EDTA, the EDTA or a salt thereof, more particularly disodium EDTA, can be present in an amount of about 0.025% to about 0.1% by weight.

One or more antioxidants can also be included in the compositions. Suitable antioxidants include, but are not limited to, ascorbic acid, sodium metabisulfite, sodium bisulfite, acetylcysteine, polyquaternium-1, benzalkonium chloride, thimerosal, chlorobutanol, methyl paraben, propyl paraben, phenylethyl alcohol, edetate disodium, sorbic acid, or other agents know to those of skill in the art. Such preservatives are typically employed at a level of from about 0.001% to about 1.0% by weight.

In some embodiments, the compounds are solubilized at least in part by an acceptable solubilizing agent. Certain acceptable nonionic surfactants, for example polysorbate 80, can be useful as solubilizing agents, as can ophthalmically acceptable glycols, polyglycols, e.g., polyethylene glycol 400 (PEG-400), and glycol ethers.

Suitable solubilizing agents for solution and solution/suspension compositions are cyclodextrins. Suitable cyclodextrins can be chosen from α-cyclodextrin, β-cyclodextrin, γ-cyclodextrin, alkylcyclodextrins (e.g., methyl-β-cyclodextrin, dimethyl-β-cyclodextrin, diethyl-β-cyclodextrin), hydroxyalkylcyclodextrins (e.g., hydroxyethyl-β-cyclodextrin, hydroxypropyl-β-cyclodextrin), carboxy-alkylcyclodextrins (e.g., carboxymethyl-β-cyclodextrin), sulfoalkylether cyclodextrins (e.g., sulfobutylether-β-cyclodextrin), and the like. Ophthalmic applications of cyclodextrins have been reviewed in Rajewski et al., Journal of Pharmaceutical Sciences, 1996, 85, 1155-1159.

In some embodiments, the composition optionally contains a suspending agent. For example, in those embodiments in which the composition is an aqueous suspension or solution/suspension, the composition can contain one or more polymers as suspending agents. Useful polymers include, but are not limited to, water-soluble polymers such as cellulosic polymers, for example, hydroxypropyl methylcellulose, and water-insoluble polymers such as cross-linked carboxyl-containing polymers.

One or more acceptable pH adjusting agents and/or buffering agents can be included in the compositions, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

One or more acceptable salts, solvates or prodrugs can be included in the compositions in an amount required to bring osmolality of the composition into an acceptable range. Such salts include, but are not limited to, those having sodium, potassium or ammonium cations and chloride, citrate, ascorbate, borate, phosphate, bicarbonate, sulfate, thiosulfate or bisulfite anions. In some embodiments, salts include sodium chloride, potassium chloride, sodium thiosulfate, sodium bisulfite and ammonium sulfate. In some embodiments, the salt is sodium chloride.

Optionally one or more acceptable surfactants, such as, but not limited to, nonionic surfactants, or co-solvents can be included in the compositions to enhance solubility of the components of the compositions or to impart physical stability, or for other purposes. Suitable nonionic surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40; polysorbate 20, 60 and 80; polyoxyethylene/polyoxypropylene surfactants (e.g., Pluronic® F-68, F84 and P-103); cyclodextrin; or other agents known to those of skill in the art. Typically, such co-solvents or surfactants are employed in the compositions at a level of from about 0.01% to about 2% by weight.

In some embodiments, pharmaceutical packs or kits comprising one or more containers filled with one or more compounds described herein are provided. Optionally associated with such container(s) can be a notice in the form prescribed by a governmental agency regulating the manufacture, use or sale of pharmaceuticals or biological products, which notice reflects approval by the agency of manufacture, use or sale for human administration for treating a condition, disease, or disorder described herein. In some embodiments, the kit contains more than one compound described herein. In some embodiments, the kit comprises a compound described herein in a single injectable dosage form, such as a single dose within an injectable device such as a syringe with a needle.

In some embodiments, the methods comprise administering to the subject one or more compounds described herein or a pharmaceutically acceptable salt, solvate or prodrug thereof, or a pharmaceutical composition of the same. In some embodiments, the subject is a subject in need of such treatment. As described herein, in some embodiments, the subject is a mammal, such as, but not limited to, a human.

In some embodiments, also provided are one or more compounds described above, or a pharmaceutically acceptable salt, solvate or prodrug thereof, or a pharmaceutical composition comprising one or more compounds described above, for use in the manufacture of a medicament for the treatment of methods of treating and/or preventing anemia associated with a ribosomal disorder, or related syndrome thereof, including, but not limited to the conditions described herein, in a subject, such as those described herein. In some embodiments, the subject is a subject in need thereof.

The present embodiments also provides the use of one or more compounds described above, or a pharmaceutically acceptable salt, solvate or prodrug thereof, or a pharmaceutical composition comprising one or more compounds described above, in the inhibition of a GlyT1 transporter, such as the presence on the surface of the cell. In some embodiments, the compounds, pharmaceutically acceptable salt thereof, or a pharmaceutical composition of the same inhibit the internalization, trafficking, and/or degradation of the GlyT1 transporter.

As used herein, “inhibition” can refer to either inhibition of a specific activity. The activity of a GlyT1 transporter can be measured by any method known in the art including but not limited to the methods described herein.

The compounds described herein are inhibitors of the GlyT1 transporter. The ability of the compounds to inhibit GlyT1 transporter activity may be measured using any assay known in the art.

Generally, assays for testing compounds that inhibit GlyT1 transporter activity include the determination of any parameter that is indirectly or directly under the influence of a GlyT1 transporter, e.g., a functional, physical, or chemical effect.

Samples or assays comprising GlyT1 transporters that are treated with a potential inhibitor, are compared to control samples without the inhibitor to examine the extent of inhibition. Control samples (untreated with inhibitors) are assigned a relative GlyT1 transporter activity value of 100%. Inhibition of a GlyT1 transporter is achieved when the GlyT1 transporter activity value relative to the control is about 80%, 50%, or 25%.

Ligand binding to a GlyT1 transporter can be tested in a number of formats. Binding can be performed in solution, in a bilayer membrane, attached to a solid phase, in a lipid monolayer, or in vesicles. For example, in an assay, the binding of the natural ligand to its transporter is measured in the presence of a candidate modulator, such as the compound described herein. Alternatively, the binding of the candidate modulator may be measured in the presence of the natural ligand. Often, competitive assays that measure the ability of a compound to compete with binding of the natural ligand to the transporter are used. Binding can be tested by measuring, e.g., changes in spectroscopic characteristics (e.g., fluorescence, absorbance, refractive index), hydrodynamic (e.g., shape) changes, or changes in chromatographic or solubility properties.

After the transporter is expressed in cells, the cells can be grown in appropriate media in the appropriate cell plate. The cells can be plated, for example at 5000-10000 cells per well in a 384 well plate. In some embodiments, the cells are plated at about 1000, 2000, 3000, 4000, 5000, 6000, 7000, 8000, 9000, or 10000 cells/per well. The plates can have any number of wells and the number of cells can be modified accordingly.

Any medicament having utility in an application described herein can be used in co-therapy, co-administration or co-formulation with a composition as described above. Therefore, the compounds described herein can be administered either before, concurrently with, or after such therapeutics are administered to a subject.

The additional medicament can be administered in co-therapy (including co-formulation) with the one or more of the compounds described herein.

In some embodiments, the response of the disease or disorder to the treatment is monitored and the treatment regimen is adjusted if necessary in light of such monitoring.

Frequency of administration is typically such that the dosing interval, for example, the period of time between one dose and the next, during waking hours is from about 1 to about 24, about 2 to about 12 hours, from about 3 to about 8 hours, or from about 4 to about 6 hours. In some embodiments, the dose is administered 1, 2, 3, or 4 times a day. It will be understood by those of skill in the art that an appropriate dosing interval is dependent to some degree on the length of time for which the selected composition is capable of maintaining a concentration of the compound(s) in the subject and/or in the target tissue (e.g., above the EC₅₀ (the minimum concentration of the compound which inhibits the transporter's activity by 90%). Ideally the concentration remains above the EC₅₀ for at least 100% of the dosing interval. Where this is not achievable it is desired that the concentration should remain above the EC₅₀ for at least about 60% of the dosing interval or should remain above the EC₅₀ for at least about 40% of the dosing interval.

### Methods of Use

The present application provides methods of preventing or treating anemia associated with a ribosomal disorder in a subject, the method comprising administering to the subject one or more glycine transporter inhibitor or a pharmaceutically acceptable salt thereof, or a prodrug of the one or more glycine transporter inhibitor or its pharmaceutically acceptable salt. In certain embodiments, the glycine transporter inhibitor is a GlyT1 inhibitor, such as a GlyT1 inhibitor as disclosed herein. For example, the present application provides a method of preventing, treating, or reducing the progression rate and/or severity of anemia associated with a ribosomal disorder in a subject, comprising administering to the subject bitopertin,

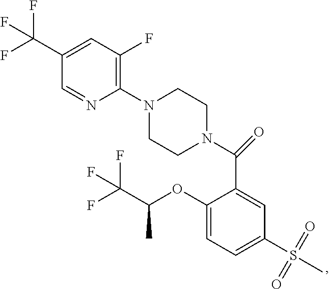 [SMILES: C[C@H](Oc1ccc(S(C)(=O)=O)cc1C(=O)N1CCN(c2ncc(C(F)(F)F)cc2F)CC1)C(F)(F)F]

or a pharmaceutically acceptable salt thereof, or a prodrug of bitopertin or its pharmaceutically acceptable salt.

In part, the present disclosure relates to methods of treating anemia associated with a ribosomal disorder in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In some embodiments, the disclosure relates to methods of preventing, treating, or reducing the progression rate and/or severity of one or more complications of anemia associated with a ribosomal disorder in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In some embodiments, the ribosomal disorder is Diamond-Blackfan anemia. In some embodiments, the ribosomal disorder is myelodysplastic syndrome associated (MDS) with isolated del(5q). In some embodiments, the ribosomal disorder is Shwachman-Diamond syndrome. In some embodiments, the ribosomal disorder is x-linked dyskeratosis congenital. In some embodiments, the ribosomal disorder is cartilage hair hypoplasia. The terms “subject,” an “individual,” or a “patient” are interchangeable throughout the specification and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

The present application provides methods of preventing, treating, or reducing the progression rate and/or severity of anemia associated with a ribosomal disorder in a subject, the method comprising administering to the subject one or more glycine transporter inhibitor, or a pharmaceutically acceptable salt thereof, or a prodrug of the one or more glycine transporter inhibitor or its pharmaceutically acceptable salt. In some embodiments, the one or more glycine transporter inhibitor is one or more GlyT1 and/or GlyT2 inhibitors. In some embodiments, the one or more glycine transporter inhibitor is one or more GlyT1 inhibitors, such as one or more GlyT1 inhibitors as disclosed herein. In certain embodiments of the foregoing, the pharmaceutical composition further comprises a pharmaceutically acceptable carrier. For example, the present application provides a method of preventing, treating, or reducing the progression rate and/or severity of anemia associated with a ribosomal disorder (e.g., Diamond-Blackfan anemia) in a subject, comprising administering to the subject bitopertin, or a pharmaceutically acceptable salt thereof, or a prodrug of bitopertin or its pharmaceutically acceptable salt.

The present application further provides use of one or more glycine transporter inhibitor, or a pharmaceutically acceptable salt thereof, or a prodrug of the one or more glycine transporter inhibitor or its pharmaceutically acceptable salt, in the manufacture of a formulation for the treatment of anemia associated with a ribosomal disorder (e.g., Diamond-Blackfan anemia) in a subject. In some embodiments, the one or more glycine transporter inhibitor is one or more GlyT1 and/or GlyT2 inhibitors. In some embodiments, the one or more glycine transporter inhibitor is one or more GlyT1 inhibitor, such as one or more GlyT1 inhibitor as disclosed herein. In certain such embodiments, the GlyT1 inhibitor is bitopertin, or a pharmaceutically acceptable salt thereof, or a prodrug of bitopertin or its pharmaceutically acceptable salt. In certain embodiments of the foregoing, the formulation is administered in a therapeutically effective amount.

### Diamond-Blackfan Anemia

Diamond-Blackfan anemia (DBA) is a congenital erythroid aplasia that usually develops during the neonatal period. DBA is characterized by low red blood cell counts (anemia) with decreased erythroid progenitors in the bone marrow. In DBA patients, levels of other blood components such as platelets and the white blood cells are normal. This is in contrast to Shwachman-Diamond syndrome, in which the bone marrow defect results primarily in low neutrophil counts (neutropenia).

Ribosomal protein mutations have been implicated in the pathophysiology of DBA. The first gene, mutated in approximately 25% of DBA patients, was identified as RPS19 (ribosomal protein S19) (Gustavsson et ah, Nat Genet. 1997 August; 16(4):368-71; Draptchinskaia et al, Nat Genet. 1999 February; 21(2): 169-75). Sequencing of patient samples has identified mutations of either large (60 s) or small (40 s) subunit ribosomal proteins in over 50% of patients (Vlachos et al, Br J Haematol. 2008 September; 142(6): 859-876). Identified genes include but are not limited to RPL5, RPL9, RPL11, RPL15, RPL17, RPL18, RPL19, RPL26, RPL27, RPL31, RPL35a, RPS7, RPS10, RPS14, RPS15a, RPS15, RPS17, RPS19, RPS20, RPS24, RPS26, RPS27a, RPS27, RPS28, and RPS29, as well as three other non-RP genes, TSR2, GATA1, and EPO (Da Costa L, et al. F1000Res. 2018; 7). All patients identified to date are heterozygous for these mutations, always maintaining a wildtype copy of the affected RP gene. However, approximately 30% of people with DBA have no detectable RP mutation. Some phenotype/genotype correlations are known, relating to congenital abnormalities. Id.

There are numerous subtypes of DBA, each of which are caused by different mutations in various genes. For instance, Diamond-Blackfan anemia-1 (DBA1, OMIM #105650) is caused by heterozygous mutations in the RPS19 gene on chromosome 19q13. Other forms of DBA include DBA2 (OMIM #606129), caused by mutations on chromosome 8p23-p22; DBA3 (OMIM #610629), caused by mutation in the RPS24 gene on 10q22; DBA4 (OMIM #612527), caused by mutation in the RPS17 gene on 15q; DBA5 (OMIM #612528), caused by mutation in the RPL35A gene on 3q29; DBA6 (OMIM #612561), caused by mutation in the RPL5 gene on 1p22. 1; DBA7 (OMIM #612562), caused by mutation in the RPL11 gene on 1p36; DBA8 (OMIM #612563), caused by mutation in the RPS7 gene on 2p25; DBA9 (OMIM #613308), caused by mutation in the RPS10 gene on 6p; DBA10 (OMIM #613309), caused by mutation in the RPS26 gene on 12q; DBA11 (OMIM #614900), caused by mutation in the RPL26 gene on 17r13; DBA12 (OMIM #615550), caused by mutation in the RPL15 gene on 3p24; DBA13 (OMIM #615909), caused by mutation in the RPS29 gene on 14q; DBA 14 (OMIM #300946), caused by mutation in the /′SR 2 gene on Xp11; DBA 15 (OMIM #606164), caused by mutation in the RPS28 gene on 19p 13; DBA 16 (OMIM #617408), caused by mutation in the RPL27 gene on chromosome 17q21; and DBA17 (OMIM #617409), caused by mutation in the RPS27 gene on chromosome 1q21.

Mutations in ribosomal proteins impact ribosomal protein function, leading to ribosomal insufficiency and increased stress. Impaired ribosome biogenesis has been linked to p53 induction and cell-cycle arrest. Ribosomal protein knockdown leads to an increase of free ribosomal proteins. Some ribosomal proteins, including RPL11, RPL5, and RPL13, bind to MDM2 and block MDM2-mediated p53 ubiquitination and degradation (Lindstrom et al, Cell Cycle 6:4, 434-437, 15 Feb. 2007; Fumagalli et al, Nat Cell Biol. 2009 April; 1 1(4):501-8). Other ribosomal proteins may activate p53 by different mechanisms. For example, RPL26 has been found to increase the translation rate of p53 mRNA by binding to its 5′ untranslated region (Tagaki et al., Cell. 2005 Oct. 7; 123(1):49-63). The negative impact of DBA on ribosomal protein function results in decreased globin synthesis, which is required to produce hemoglobin. Heme synthesis does not appear to be impacted. The imbalance between heme synthesis and globin leads to the accumulation of free heme in DBA erythroid cells (Rio S, et al. Blood. 2019; 133(12):1358-1370). Heme is toxic for the cells by increasing reactive oxygen species production, lipid peroxidation, and apoptosis. As a consequence, excess heme levels resulting from the heme/globin imbalance leads to deleterious effects on erythroipoiesis.

Typically, a diagnosis of DBA is made through a blood count and a bone marrow biopsy. A diagnosis of DBA is made on the basis of anemia, low reticulocyte (immature red blood cells) counts, and diminished erythroid precursors in bone marrow. Features that support a diagnosis of DBA include the presence of congenital abnormalities, macrocytosis, elevated fetal hemoglobin, and elevated adenosine deaminase levels in red blood cells. Most patients are diagnosed in the first two years of life. However, some mildly affected individuals only receive attention after a more severely affected family member is identified. Genetic testing is frequently used to identify mutations in ribosomal protein genes as well as some other non-ribosomal protein genes. About 20-25% of DBA patients may be identified with a genetic test for mutations in the RPS19 gene. Approximately 10-25% of DBA cases have a family history of disease, and most pedigrees suggest an autosomal dominant mode of inheritance.

In certain aspects, the disclosure relates to methods of treating anemia associated with a ribosomal disorder in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In certain aspects, the disclosure relates to methods of preventing, treating, or reducing the progression rate and/or severity of anemia associated with a ribosomal disorder in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In some embodiments, the anemia associated with a ribosomal disorder is Diamond Blackfan anemia (DBA). In some embodiments, the DBA is caused by haploinsufficiency for a ribosomal protein selected from the group consisting of 40S ribosomal protein S14 (RPS14), 40S ribosomal protein S19 (RPS19), 40S ribosomal protein S24 (RPS24), 40S ribosomal protein S17 (RPS17), 60S ribosomal protein L35a (RPL35a), 60S ribosomal protein L5 (RPL5), 60S ribosomal protein L11 (RPL11), and 40S ribosomal protein S7 (RPS7).). In some embodiments, the DBA is caused by haploinsufficiency for a ribosomal protein selected from the group consisting of 40S ribosomal protein S10 (RPS10), 40S ribosomal protein S26 (RPS26), 60S ribosomal protein L15 (RPL15), 60S ribosomal protein L17 (RPL17), 60S ribosomal protein L19 (RPL19), 60S ribosomal protein L26 (RPL26), 60S ribosomal protein L27 (RPL27), 60S ribosomal protein L31 (RPL31), 40S ribosomal protein S15a (RPS15a), 40S ribosomal protein S20 (RPS20), 40S ribosomal protein S27 (RPS27), 40S ribosomal protein S28 (RPS28), and 40S ribosomal protein S29 (RPS29). In some embodiments, the patient has one or more mutations in a ribosomal protein gene.

In some embodiments, the GlyT1 inhibitors as disclosed herein can be used in a method of treating anemia associated with a ribosomal disorder, wherein the subject has a mutation in ribosomal protein 19 (RPS19). The phenotype of DBA patients indicates a hematological stem cell defect specifically affecting the erythroid progenitor population. The RPS19 protein is involved in the production of ribosomes. Disease features may be related to the nature of RPS19 mutations. The disease is characterized by dominant inheritance, and therefore arises due to a partial loss of RPS19 protein function.

In alternative embodiments, the GlyT1 inhibitors as disclosed herein can be used in a method of treating anemia associated with a ribosomal disorder, wherein the subject has a mutation in ribosomal protein from at least one of, but not limited to RPL5, RPL9, RPL11, RPL15, RPL17, RPL18, RPL19, RPL26, RPL27, RPL31, RPL35a, RPS7, RPS10, RPS14, RPS15a, RPS15, RPS17, RPS19, RPS20, RPS24, RPS26, RPS27a, RPS27, RPS28, and RPS29. For example, a mutation or variant in RPS19 causes DBA1, and a mutation or variant in RPS24 causes DBA3, a mutation or variant in RPS17 causes DBA4, a mutation or variant in RPS34A causes DBA5, a mutation or variant in RPLS causes DBA6, a mutation or variant in RPL11 causes DBA7, and a mutation or variant in RPS7 causes DBA8. In some embodiments, the subject with a ribosomal disorder has a mutation in a non-ribosomal protein selected from the group consisting of TSR2, GATA1, and EPO.

In certain aspects, the disclosure relates to methods of preventing, treating, or reducing the progression rate and/or severity of one or more complications of anemia associated with a ribosomal disorder in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In some embodiments, the one or more complications of anemia associated with a ribosomal disorder is selected from the group consisting of: thrombocytosis, megakaryotypic hyperplasia, infections, bleeding (e.g., from the nose or gums), bruising, splenomegaly, the need for more frequent blood transfusions, the need for increased glucocorticoid use, the need for allogenic hematopoietic stem cell transplantation, the need for autologous gene therapy, marrow failure, MDS, leukemia, and acute myelogenous leukemia.

In certain aspects, the disclosure relates to methods of treating splenomegaly associated with anemia associated with a ribosomal disorder in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In some embodiments, the subject has an increased spleen size (e.g., splenomegaly). In some embodiments, the GlyT1 inhibitors disclosed herein reduce splenomegaly in a subject with anemia associated with a ribosomal disorder (e.g., Diamond-Blackfan anemia). In some embodiments, the method reduces the subject's spleen size. In some embodiments, the method reduces the subject's spleen size by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method reduces the subject's spleen size by at least 15%. In some embodiments, the method reduces the subject's spleen size by at least 20%. In some embodiments, the method reduces the subject's spleen size by at least 25%. In some embodiments, the method reduces the subject's spleen size by at least 30%. In some embodiments, the method reduces the subject's spleen size by at least 35%. In some embodiments, the method reduces the subject's spleen size by at least 40%. In some embodiments, the method reduces the subject's spleen size by at least 45%. In some embodiments, the method reduces the subject's spleen size by at least 50%. In some embodiments, the method reduces the subject's spleen size by at least 55%. In some embodiments, the method reduces the subject's spleen size by at least 60%. In some embodiments, the method reduces the subject's spleen size by at least 65%. In some embodiments, the method reduces the subject's spleen size by at least 70%. In some embodiments, the method reduces the subject's spleen size by at least 75%. In some embodiments, the method reduces the subject's spleen size by at least 80%. In some embodiments, the method reduces the subject's spleen size by at least 85%. In some embodiments, the method reduces the subject's spleen size by at least 90%. In some embodiments, the method reduces the subject's spleen size by at least 95%. In some embodiments, the method reduces the subject's spleen size by at least 100%.

In some embodiments, the methods and GlyT1 inhibitors as disclosed herein can be used to treat a subject with a ribosomal disorder, such as DBA, wherein the subject has a symptom of macrocytic anemia and/or craniofacial abnormalities.

### Myelodysplasia

Myelodysplasia or myelodysplastic syndromes (MDS) are a group of hematological disorders related to the body's inability to produce enough normal blood cells. In MDS patients, the immature blood cells in the bone marrow do not mature and instead they die in the bone marrow or just after entering the bloodstream. MDS can affect the production of any, and sometimes all, types of blood cells including red blood cells, platelets, and white blood cells (cytopenias). Over time, there are more immature, defective cells than healthy ones. As a result, patients with MDS often have anemia (low red blood cell count or reduced hemoglobin) which can cause fatigue and shortness of breath, neutropenia (low neutrophil count) which can cause increased susceptibility to infection, and/or thrombocytopenia (low platelet count) which can cause bleeding and easy bruising with no apparent cause.

Marrow cell disturbances in MDS patients range from mild to very severe. In some cases, patients with MDS often develop severe anemia and require frequent blood transfusions. In most cases, the disease worsens and the patient develops cytopenias caused by progressive bone marrow failure. In about 30% of patients with MDS, the disease progresses into acute myelogenous leukemia (AML), usually within months to a few years.

There are multiple prognostic scoring systems which use prognostic indicators to predict the course of the patient's disease. These include the International Prognostic Scoring System (IPSS), the Revised International Prognostic Scoring System (IPSS-R), and the WHO classification-based Prognostic Scoring System (WPSS). The IPSS is the most commonly used prognostic scoring system and it uses the following three prognostic indicators to predict the course of the patients disease: (1) the percentage of leukemic blast cells in the marrow; (2) the type of chromosomal changes, if any, in the marrow cells (cytogenetics); and (3) the presence of one or more low blood cell counts (cytopenias).

Prognostic Factors Scored

Percent of blast cells in bone marrow:
Less than 5 = 0 points
5 to 10 = 0.5 points
11 to 20 = 1.5 points
21 to 30 = 2 points
Cytogenetics (chromosome changes):
None, del(5q), del(20q) = 0 points
3 or more abnormalities, abnormal chromosome 7 = 1 point
Other abnormalities = 0.5 points
Number of cytopenias (anemia, neutropenia or thrombocytopenia):
None or 1 = 0 points
2 or 3 = 0.5 points

The risk groups in the IPSS are based upon the point totals for each of the above prognostic factors. The overall risk score indicates how fast the disease is likely to progress and doctors often use the system to assign the patient to a particular risk group. Patients having 0 points are considered low risk. Patients with between 0.5 to 1 points are considered Intermediate-1 risk. Patients with between 1.5 to 2 points are considered Intermediate-2 risk. Finally, patients with 2.5 or more points are considered high risk.

MDS most often affect adults between the age of 60 and 75 years. MDS in children is rare. Males are slightly more commonly affected than females. Previous treatment with chemotherapy or radiation is a key factor in the onset of MDS. Exposure to certain chemicals (e.g., tobacco smoke, pesticides, benzene) and heavy metals (e.g., lead, mercury) can increase the risk of myelodysplastic syndromes. Some inherited disorders can also lead to MDS, including Shwachman-Diamond syndrome and Diamond-Blackfan anemia.

Myelodysplastic Syndrome Associated with Isolated Del(5q)

Myelodysplastic syndrome associated (MDS) with isolated del(5q), also known as 5q-myelodysplasia, Del 5q, 5q-syndrome, chromosome 5q deletion syndrome, or chromosome 5q monosomy, is a rare form of MDS. It is caused by deletion of a region of DNA in the long arm (q arm, band 5q31.1) of human chromosome 5. Most people with MDS with isolated del(5q) are missing a fragment of about 1.5 million base pairs. MDS with isolated del(5q) is characterized by severe anemia, frequent thrombocytosis, typical dysmegakaryopoiesis and favorable outcome. Unlike other MDS types, MDS with isolated del(5q) is found predominantly in females of advanced age.

The commonly deleted region of DNA in MDS with isolated del(5q) contains 40 genes, including RPS14, MIR145 and MIR146 loci. Loss of the RPS14 gene leads to the problems with red blood cell development characteristic of MDS with isolated del(5q), and loss of the MIR 145 and MIR146 loci contributes to the platelet abnormalities and megakaryocyte dysplasia associated with the MDS with isolated del(5q).

Subjects with MDS with isolated del(5q) can be treated with lenalidomide (REVLIMID®) (Bennett et al., N Engl J Med. 2006 Oct. 5; 355(14): 1456-65; Raza et al., Blood. 2008 Jan. 1; 111(1):86-93). One of the side effects of lenalidomide treatment may be low blood cell counts initially leading the individual to utilize supportive care. Supportive care includes red blood cell transfusion, antibiotics, and Iron chelation therapy. For younger people, bone marrow transplantation is an option and is the only known cure for MDS.

In certain aspects, the disclosure relates to methods of treating MDS with isolated del(5q) in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In certain aspects, the disclosure relates to methods of preventing, treating, or reducing the progression rate and/or severity of MDS with isolated del(5q) in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In some embodiments, the subject has low risk MDS as classified by the IPSS. In some embodiments, the subject has intermediate-1 risk MDS as classified by the IPSS. In some embodiments, the subject has intermediate-2 risk MDS as classified by the IPSS. In some embodiments, the subject has high risk MDS as classified by the IPSS. In some embodiments, the subject is haploinsufficient for a ribosomal protein selected from the group consisting of 40S ribosomal protein S14 (RPS14) and 40S ribosomal protein S19 (RPS19). In some embodiments, the subject has impaired 40S ribosomal subunit maturation. In some embodiments, the subject has impaired 60S ribosomal subunit maturation. In some embodiments, the subject has one or more mutations in a ribosomal protein gene. In some embodiments, the one or more mutations in a ribosomal protein gene are selected from the group consisting of RPS14 or RPS19.

In certain aspects, the disclosure contemplates the use of a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt, in combination with one or more additional active agents or other supportive therapy for treating or preventing anemia associated with a ribosomal disorder. In some embodiments, the glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt is administered in combination with lenalidomide (REVLIMID®).

### Shwachman-Diamond Syndrome

Shwachman-Diamond syndrome (SDS) or Shwachman-Bodian-Diamond syndrome is a rare genetic disorder that that affects many parts of the body, particularly the pancreas, bone marrow, and skeletal system. Shwachman-Diamond syndrome is inheritated in an autosomal recessive pattern. Most cases of SDS are caused by mutations in the SBDS gene, which lies on the long arm of chromosome 7 at cytogenetic position 7q11. The protein encoded by SBDS is thought to play a role in RNA processing and ribosome biogenesis, although the exact mechanism of how SBDS mutations lead to the major signs and symptoms of Shwachman-Diamond syndrome is still unclear. Typical symptoms of Shwachman-Diamond syndrome include exocrine pancreatic insufficiency, decreased muscle tone, low blood neutrophil count (neutropenia), anemia, and abnormal bone development affecting the rib cage and/or bones in the arms and/or legs (metaphyseal dysostosis).

Diagnosis of Shwachman-Diamond syndrome can be made based on clinical findings, including pancreatic dysfunction and characteristic hematologic abnormalities (e.g., neutropenia and thrombocytopenia). Genetic testing may be used to confirm the diagnosis. SBDS gene mutations are known to cause about 90% of cases of Shwachman-Diamond syndrome. The remaining 10% cases have unknown genetic cause, and hence genetic testing is not an option for these cases.

There is no cure for Shwachman-Diamond syndrome. Treatment usually include oral pancreatic enzyme replacement, vitamin supplementation, blood and/or platelet transfusion, administration of granulocyte-colony stimulating factor (G-CSF), and/or hematopoietic stem cell transplantation. The shortage of neutrophils in subjects with Shwachman-Diamond syndrome can lead to neutropenia, which makes them more vulnerable to infections such as pneumonia. Patients with Shwachman-Diamond syndrome also have a higher than average chance of developing MDS, aplastic anemia, and leukemia (e.g., acute myeloid leukemia).

In certain aspects, the disclosure relates to methods of treating Shwachman-Diamond syndrome in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In certain aspects, the disclosure relates to methods of preventing, treating, or reducing the progression rate and/or severity of Shwachman-Diamond syndrome in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In some embodiments, the subject has one or more mutations in the SBDS gene. In some embodiments, the method decreases the need for hematopoietic stem cell transplant in the subject. In some embodiments, the method decreases neutropenia in the subject. In some embodiments, the method decreases thrombocytopenia in the subject. In some embodiments, the method decreases the subject's risk of developing myelodysplastic syndrome. In some embodiments, the method decreases the subject's risk of developing leukemia. In some embodiments, the method decreases the subject's risk of developing an infection. In some embodiments, the method decreases the subject's risk of developing pneumonia. In some embodiments, the subject has low neutrophil levels.

### Dyskeratosis Congenita

Dyskeratosis congenita, also known as Zinsser-Engman-Cole syndrome, is a rare genetic form of bone marrow failure which is classically associated with oral leukoplakia, nail dystrophy, and reticular hyperpigmentation. Inheritance is most commonly x-linked recessive. As such, males are three times more likely to be affected than females. Symptoms vary widely and may include atrophic wrinkled skin, eye disease, and bone marrow failure. Dyskeratosis congenita patients are at increased risk of developing leukemia and other cancers (e.g., cancers of the head, neck, anus, or genitals) as well as fibrosis (e.g., pulmonary fibrosis and liver fibrosis).

The majority of patients have mutations in dyskerin gene (DKC1), a protein which is directly involved in stabilizing an enzyme called telomerase that is responsible for catalyzing a reaction that sustains the length of telomeres. Without proteins like dyskerin, the telomeres progressively shorten casing the cells to undergo apoptosis or senescence. Other genes including TINF2, TERC, TERT, C16orf57, NOLA2, NOLA3, WRAP53/TCAB1, and RTEL1 have been shown to be mutated in dyskeratosis congenita.

Treatment options for patients with dyskeratosis congenita are limited. The only long-term treatment option for bone failure in dyskeratosis congenita patients is hematopoietic stem cell transplantation. However, long-term outcomes remain poor, with an estimated 10-year survival rate of 23%. Short-term treatment options include anabolic steroids (e.g., oxymetholone), granulocyte macrophage colony-stimulating factor, granulocyte colony-stimulating factor, and erythropoietin.

In certain aspects, the disclosure relates to methods of treating dyskeratosis congenita in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In certain aspects, the disclosure relates to methods of preventing, treating, or reducing the progression rate and/or severity of dyskeratosis congenita in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In some embodiments, the subject has dyskeratosis congenita. In some embodiments, the dyskeratosis congenita is x-linked dyskeratosis congenita. In some embodiments, the subject has one or more mutations in the DKC1 gene. In some embodiments, the subject has one or more mutations in a gene selected from the group consisting of TINF2, TERC, TERT, C16orf57, NOLA2, NOLA3, WRAP53/TCAB1, PARN, CTC1, and RTEL1. In some embodiments, the method decreases the risk of bone marrow failure in the subject. In some embodiments, the method decreases the risk of pulmonary fibrosis in the subject. In some embodiments, the method decreases the risk of liver fibrosis in the subject.

### Cartilage-Hair Hypoplasia

Cartilage-hair hypoplasia, also known as McKusick type metaphyseal chondrodysplasia, is a disorder of bone growth characterized by short stature (dwarfism) with other skeletal abnormalities; fine, sparse hair; joint hypermobility; anemia; increased risk for malignancy; gastrointestinal dysfunction; impaired spermatogenesis; and abnormal immune system function which often leads to recurrent infections. Patients with cartilage-hair hypoplasia. Most patients with cartilage-hair hypoplasia have a mutation in the RMRP gene (OMIM no. 157660), with a 70A→G transition mutation commonly present. The RMRP gene encodes the untranslated RNA component of the mitochondrial RNA-processing ribonuclease, RNase MRP.

Diagnosis of cartilage-hair hypoplasia is based primarily on clinical findings, characteristic radiographic findings, and in some cases, evidence of immune dysfunction, macrocytic anemia, and/or gastrointestinal problems. Molecular genetic testing can be used in patients to identify pathogenic variants by RMRP.

Treatment of patients often incudes repeated blood transfusions and surgeries to fuse unstable vertebrae or to treat progressive kyphoscoliosis which compromises lung function. Corrective osteotomies may also be required to treat progressive varus deformity associated with ligament laxity in the knees. For patients with immunodeficiency, frequent treatments of underlying infections is required. Prophylatic antibiotic therapy and/or immunoglobulin replacement therapy is often required. Recurrent severe infections and/or the presence of severe combined immunodeficiency (SCID) and/or severely depressed erythropoiesis may warrant bone marrow transplantation.

In certain aspects, the disclosure relates to methods of treating cartilage-hair hypoplasia in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In certain aspects, the disclosure relates to methods of preventing, treating, or reducing the progression rate and/or severity of cartilage-hair hypoplasia in a subject, the method comprising administering to the subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In some embodiments, the subject has one or more mutations in the RMRP gene. In some embodiments, the method reduces the need for bone marrow transplantation in the subject.

### Defects in Erythropoiesis

Erythropoiesis refers generally to the process by which red blood cells (erythrocytes) are produced from HSCs, and includes the formation of erythroid progenitor cells. Erythropoiesis is a carefully ordered sequence of events. Initially occurring in fetal hepatocytes, the process is taken over by the bone marrow in the child and adult. Although multiple cytokines and growth factors are dedicated to the proliferation of the red blood cell, the primary regulator is erythropoietin (EPO). Red blood cell development is initially regulated by stem cell factor (SCF), which commits hematopoietic stem cells to develop into erythroid progenitors. Subsequently, EPO continues to stimulate the development and terminal differentiation of these progenitors. In the fetus, EPO is produced by monocytes and macrophages found in the liver. After birth, EPO is produced in the kidneys; however, Epo messenger RNA (mRNA) and EPO protein are also found in the brain and in red blood cells (RBCs), suggesting the presence of paracrine and autocrine functions.

Erythropoiesis escalates as increased expression of the EPO gene produces higher levels of circulating EPO. EPO gene expression is known to be affected by multiple factors, including hypoxemia, transition metals (Co2+, Ni2+, Mn2+), and iron chelators. However, the major influence is hypoxia, including factors of decreased oxygen tension, red blood cell loss, and increased oxygen affinity of hemoglobin. For instance, EPO production may increase as much as 1000-fold in severe hypoxia.

Erythropoiesis requires the proper biosynthesis of heme and as erythroblasts mature, their demand for heme and iron dramatically increase. Erythroid cells synthesize large amounts of heme and hemoglobin while simultaneously absorbing lots of iron into the cell. A disequilibrium between the globin chain and the heme synthesis is known to occur in the erythroid cells of Diamond-Blackfan anemia patients. This imbalance leads to the accumulation of excess free heme and increased reactive oxygen species production.

Blockade of erythroid differentiation and proliferation in Diamond-Blackfan anemia have been shown to affect immature progenitor cells or erythroid-Burst-Forming Unit (BFU-e) resulting in impaired hematopoiesis. Circulating EPO levels are increased in Diamond-Blackfan anemia patients, indicating the unresponsiveness of the bone marrow to anemia related EPO stimulation. An increased propensity of erythroid progenitors to apoptosis during in vitro EPO deprivation and in RPS19 deficiency has also been reported.

Glycine is one of the key initial substrates for heme synthesis. As such, decreased levels of glycine due to GlyT1 inhibition could lead to a decrease in heme synthesis. In certain aspects, the disclosure relates to methods of inhibiting heme synthesis in a subject with anemia associated with a ribosomal disorder, comprising administering to a subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In some embodiments, the heme synthesis is inhibited in a dose dependent manner.

In some embodiments, the subject with anemia associated with a ribosomal disorder (e.g., Diamond-Blackfan anemia) has elevated heme levels. In some embodiments, the subject has heme levels that are at least 10%, 20%, 30%, 40%, 50%, 60%, 70%, 80%, 90% or 100% more than heme levels in a healthy subject prior to administration of the GlyT1 inhibitor. In some embodiments, the subject has heme levels that are at least 10% more than heme levels in a healthy subject prior to administration of the GlyT1 inhibitor. In some embodiments, the subject has heme levels that are at least 20% more than heme levels in a healthy subject prior to administration of the GlyT1 inhibitor. In some embodiments, the subject has heme levels that are at least 30% more than heme levels in a healthy subject prior to administration of the GlyT1 inhibitor. In some embodiments, the subject has heme levels that are at least 40% more than heme levels in a healthy subject prior to administration of the GlyT1 inhibitor. In some embodiments, the subject has heme levels that are at least 50% more than heme levels in a healthy subject prior to administration of the GlyT1 inhibitor. In some embodiments, the subject has heme levels that are at least 60% more than heme levels in a healthy subject prior to administration of the GlyT1 inhibitor. In some embodiments, the subject has heme levels that are at least 70% more than heme levels in a healthy subject prior to administration of the GlyT1 inhibitor. In some embodiments, the subject has heme levels that are at least 80% more than heme levels in a healthy subject prior to administration of the GlyT1 inhibitor. In some embodiments, the subject has heme levels that are at least 90% more than heme levels in a healthy subject prior to administration of the GlyT1 inhibitor. In some embodiments, the subject has heme levels that are at least 100% more than heme levels in a healthy subject prior to administration of the GlyT1 inhibitor.

In some embodiments, the method reduces the heme levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method reduces the heme levels in the subject by at least 15%. In some embodiments, the method reduces the heme levels in the subject by at least 20%. In some embodiments, the method reduces the heme levels in the subject by at least 25%. In some embodiments, the method reduces the heme levels in the subject by at least 30%. In some embodiments, the method reduces the heme levels in the subject by at least 35%. In some embodiments, the method reduces the heme levels in the subject by at least 40%. In some embodiments, the method reduces the heme levels in the subject by at least 45%. In some embodiments, the method reduces the heme levels in the subject by at least 50%. In some embodiments, the method reduces the heme levels in the subject by at least 55%. In some embodiments, the method reduces the heme levels in the subject by at least 60%. In some embodiments, the method reduces the heme levels in the subject by at least 65%. In some embodiments, the method reduces the heme levels in the subject by at least 70%. In some embodiments, the method reduces the heme levels in the subject by at least 75%. In some embodiments, the method reduces the heme levels in the subject by at least 80%. In some embodiments, the method reduces the heme levels in the subject by at least 85%. In some embodiments, the method reduces the heme levels in the subject by at least 90%. In some embodiments, the method reduces the heme levels in the subject by at least 95%. In some embodiments, the method reduces the heme levels in the subject by at least 100%.

In some embodiments, the method reduces heme synthesis in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method reduces heme synthesis in the subject by at least 15%. In some embodiments, the method reduces heme synthesis in the subject by at least 20%. In some embodiments, the method reduces heme synthesis in the subject by at least 25%. In some embodiments, the method reduces heme synthesis in the subject by at least 30%. In some embodiments, the method reduces heme synthesis in the subject by at least 35%. In some embodiments, the method reduces heme synthesis in the subject by at least 40%. In some embodiments, the method reduces heme synthesis in the subject by at least 45%. In some embodiments, the method reduces heme synthesis in the subject by at least 50%. In some embodiments, the method reduces heme synthesis in the subject by at least 55%. In some embodiments, the method reduces heme synthesis in the subject by at least 60%. In some embodiments, the method reduces heme synthesis in the subject by at least 65%. In some embodiments, the method reduces heme synthesis in the subject by at least 70%. In some embodiments, the method reduces heme synthesis in the subject by at least 75%. In some embodiments, the method reduces heme synthesis in the subject by at least 80%. In some embodiments, the method reduces heme synthesis in the subject by at least 85%. In some embodiments, the method reduces heme synthesis in the subject by at least 90%. In some embodiments, the method reduces heme synthesis in the subject by at least 95%. In some embodiments, the method reduces heme synthesis in the subject by at least 100%. In some embodiments, the method reduces intracellular heme levels. In some embodiments, the method reduces intracellular heme levels in erythroid precursors.

In some embodiments, the method reduces the risk of heme toxicity in the subject. In some embodiments, the method reduces the risk of heme toxicity in the subject by at least 10% (e.g., 10%, 15%, 20%, 25%, 30%, 35%, 40%, 45%, 50%, 55% 60%, 605%, 70%, 75%, 80%, 85%, 90%, 95%, or at least 100%). In some embodiments, the method reduces the risk of heme toxicity in the subject by at least 15%. In some embodiments, the method reduces the risk of heme toxicity in the subject by at least 20%. In some embodiments, the method reduces the risk of heme toxicity in the subject by at least 25%. In some embodiments, the method reduces the risk of heme toxicity in the subject by at least 30%. In some embodiments, the method reduces the risk of heme toxicity in the subject by at least 35%. In some embodiments, the method reduces the risk of heme toxicity in the subject by at least 40%. In some embodiments, the method reduces the risk of heme toxicity in the subject by at least 45%. In some embodiments, the method reduces the risk of heme toxicity in the subject by at least 50%. In some embodiments, the method reduces the risk of heme toxicity in the subject by at least 55%. In some embodiments, the method reduces the risk of heme toxicity in the subject by at least 60%. In some embodiments, the method reduces the risk of heme toxicity in the subject by at least 65%. In some embodiments, the method reduces the risk of heme toxicity in the subject by at least 70%. In some embodiments, the method reduces the risk of heme toxicity in the subject by at least 75%. In some embodiments, the method reduces the risk of heme toxicity in the subject by at least 80%. In some embodiments, the method reduces the risk of heme toxicity in the subject by at least 85%. In some embodiments, the method reduces the risk of heme toxicity in the subject by at least 90%. In some embodiments, the method reduces the risk of heme toxicity in the subject by at least 95%. In some embodiments, the method reduces the risk of heme toxicity in the subject by at least 100%.

In some embodiments, the subject has liver iron overload. In some embodiments, the method reduces the risk of liver iron overload. In some embodiments, the method reduces the levels of iron in the liver. In some embodiments, the method reduces the levels of iron in the liver by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method reduces the levels of iron in the liver by at least 15%. In some embodiments, the method reduces the levels of iron in the liver by at least 20%. In some embodiments, the method reduces the levels of iron in the liver by at least 25%. In some embodiments, the method reduces the levels of iron in the liver by at least 30%. In some embodiments, the method reduces the levels of iron in the liver by at least 35%. In some embodiments, the method reduces the levels of iron in the liver by at least 40%. In some embodiments, the method reduces the levels of iron in the liver by at least 45%. In some embodiments, the method reduces the levels of iron in the liver by at least 50%. In some embodiments, the method reduces the levels of iron in the liver by at least 55%. In some embodiments, the method reduces the levels of iron in the liver by at least 60%. In some embodiments, the method reduces the levels of iron in the liver by at least 65%. In some embodiments, the method reduces the levels of iron in the liver by at least 70%. In some embodiments, the method reduces the levels of iron in the liver by at least 75%. In some embodiments, the method reduces the levels of iron in the liver by at least 80%. In some embodiments, the method reduces the levels of iron in the liver by at least 85%. In some embodiments, the method reduces the levels of iron in the liver by at least 90%. In some embodiments, the method reduces the levels of iron in the liver by at least 95%. In some embodiments, the method reduces the levels of iron in the liver by at least 100%.

In some embodiments, the subject has cardiac iron overload. In some embodiments, the method reduces the risk of cardiac iron overload. In some embodiments, the method reduces the level of iron in the heart. In some embodiments, the method reduces the levels of iron in the heart by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method reduces the levels of iron in the heart by at least 15%. In some embodiments, the method reduces the levels of iron in the heart by at least 20%. In some embodiments, the method reduces the levels of iron in the heart by at least 25%. In some embodiments, the method reduces the levels of iron in the heart by at least 30%. In some embodiments, the method reduces the levels of iron in the heart by at least 35%. In some embodiments, the method reduces the levels of iron in the heart by at least 40%. In some embodiments, the method reduces the levels of iron in the heart by at least 45%. In some embodiments, the method reduces the levels of iron in the heart by at least 50%. In some embodiments, the method reduces the levels of iron in the heart by at least 55%. In some embodiments, the method reduces the levels of iron in the heart by at least 60%. In some embodiments, the method reduces the levels of iron in the heart by at least 65%. In some embodiments, the method reduces the levels of iron in the heart by at least 70%. In some embodiments, the method reduces the levels of iron in the heart by at least 75%. In some embodiments, the method reduces the levels of iron in the heart by at least 80%. In some embodiments, the method reduces the levels of iron in the heart by at least 85%. In some embodiments, the method reduces the levels of iron in the heart by at least 90%. In some embodiments, the method reduces the levels of iron in the heart by at least 95%. In some embodiments, the method reduces the levels of iron in the heart by at least 100%.

In some embodiments, the subject has decreased erythroid precursor survival as compared to a healthy subject. In some embodiments, the subject has decreased erythroid precursor differentiation into mature red blood cells as compared to a healthy subject. In some embodiments, the subject has impaired hematopoiesis. In some embodiments, the method increases the subject's erythroid precursor survival. In some embodiments, the method increases the subject's erythroid precursor survival by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method increases the subject's erythroid precursor survival by at least 15%. In some embodiments, the method increases the subject's erythroid precursor survival by at least 20%. In some embodiments, the method increases the subject's erythroid precursor survival by at least 25%. In some embodiments, the method increases the subject's erythroid precursor survival by at least 30%. In some embodiments, the method increases the subject's erythroid precursor survival by at least 35%. In some embodiments, the method increases the subject's erythroid precursor survival by at least 40%. In some embodiments, the method increases the subject's erythroid precursor survival by at least 45%. In some embodiments, the method increases the subject's erythroid precursor survival by at least 50%. In some embodiments, the method increases the subject's erythroid precursor survival by at least 55%. In some embodiments, the method increases the subject's erythroid precursor survival by at least 60%. In some embodiments, the method increases the subject's erythroid precursor survival by at least 65%. In some embodiments, the method increases the subject's erythroid precursor survival by at least 70%. In some embodiments, the method increases the subject's erythroid precursor survival by at least 75%. In some embodiments, the method increases the subject's erythroid precursor survival by at least 80%. In some embodiments, the method increases the subject's erythroid precursor survival by at least 85%. In some embodiments, the method increases the subject's erythroid precursor survival by at least 90%. In some embodiments, the method increases the subject's erythroid precursor survival by at least 95%. In some embodiments, the method increases the subject's erythroid precursor survival by at least 100%.

In some embodiments, the method increases erythroid precursor differentiation into mature red blood cells in the subject. In some embodiments, the method increases erythroid precursor differentiation into mature red blood cells in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method increases erythroid precursor differentiation into mature red blood cells by at least 15%. In some embodiments, the method increases erythroid precursor differentiation into mature red blood cells by at least 20%. In some embodiments, the method increases erythroid precursor differentiation into mature red blood cells by at least 25%. In some embodiments, the method increases erythroid precursor differentiation into mature red blood cells by at least 30%. In some embodiments, the method increases erythroid precursor differentiation into mature red blood cells by at least 35%. In some embodiments, the method increases erythroid precursor differentiation into mature red blood cells by at least 40%. In some embodiments, the method increases erythroid precursor differentiation into mature red blood cells by at least 45%. In some embodiments, the method increases erythroid precursor differentiation into mature red blood cells by at least 50%. In some embodiments, the method increases erythroid precursor differentiation into mature red blood cells by at least 55%. In some embodiments, the method increases erythroid precursor differentiation into mature red blood cells by at least 60%. In some embodiments, the method increases erythroid precursor differentiation into mature red blood cells by at least 65%. In some embodiments, the method increases erythroid precursor differentiation into mature red blood cells by at least 70%. In some embodiments, the method increases erythroid precursor differentiation into mature red blood cells by at least 75%. In some embodiments, the method increases erythroid precursor differentiation into mature red blood cells by at least 80%. In some embodiments, the method increases erythroid precursor differentiation into mature red blood cells by at least 85%. In some embodiments, the method increases erythroid precursor differentiation into mature red blood cells by at least 90%. In some embodiments, the method increases erythroid precursor differentiation into mature red blood cells by at least 95%. In some embodiments, the method increases erythroid precursor differentiation into mature red blood cells by at least 100%. In some embodiments, the subject has elevated erythrocyte adenosine deaminase activity. In some embodiments, the subject has normal marrow cellularity with a paucity of red cell precursors. In some embodiments, the subject has normal neutrophil and/or platelet counts.

In some embodiments, the anemia is due to a failure in erythropoiesis. In some embodiments, the method reduces anemia in the subject. In some embodiments, the method reduces anemia in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method reduces anemia in the subject by at least 15%. In some embodiments, the method reduces anemia in the subject by at least 20%. In some embodiments, the method reduces anemia in the subject by at least 25%. In some embodiments, the method reduces anemia in the subject by at least 30%. In some embodiments, the method reduces anemia in the subject by at least 35%. In some embodiments, the method reduces anemia in the subject by at least 40%. In some embodiments, the method reduces anemia in the subject by at least 45%. In some embodiments, the method reduces anemia in the subject by at least 50%. In some embodiments, the method reduces anemia in the subject by at least 55%. In some embodiments, the method reduces anemia in the subject by at least 60%. In some embodiments, the method reduces anemia in the subject by at least 65%. In some embodiments, the method reduces anemia in the subject by at least 70%. In some embodiments, the method reduces anemia in the subject by at least 75%. In some embodiments, the method reduces anemia in the subject by at least 80%. In some embodiments, the method reduces anemia in the subject by at least 85%. In some embodiments, the method reduces anemia in the subject by at least 90%. In some embodiments, the method reduces anemia in the subject by at least 95%. In some embodiments, the method reduces anemia in the subject by at least 100%. In some embodiments, the subject has macrocytic anemia. In some embodiments, the method reduces anemia in the subject by reducing free heme toxicity.

In some embodiments, the method increases red cell mass. In some embodiments, the method decreases the mean corpuscular volume of red cells. In some embodiments, the method decreases red cell adenosine deaminase. In some embodiments, the method decreases red cell adenosine deaminase in a subject with DBA. In some embodiments, the method decreases fetal hemoglobin content in red cells.

### Red Blood Cell Count and Hematocrit

Certain embodiments of the present disclosure relate to methods of administering a GlyT1 inhibitor disclosed herein to a subject in need thereof, wherein the subject has an low red blood cell count (e.g., less than about 4.5 million red blood cells per μl of blood for men and about 4.1 million red blood cells per μl of blood for women, often by a clinically or statistically significant amount), or a low hematocrit (e.g., greater than about 38% for men or about 35% for women, often by a clinically or statistically significant amount). In some embodiments, the subject has hematocrit levels that are less than 38%. In some embodiments, the subject has hematocrit levels that are less than 35%.

In some embodiments, the subject's hematocrit levels are at least 10%, 20%, 30%, 40%, 50%, 60%, 70%, 80%, or 90% less than hematocrit levels in a healthy subject prior to administration of the GlyT1 inhibitor. In some embodiments, the subject's hematocrit levels are at least 10% less than hematocrit levels in a healthy subject prior to administration of the GlyT1 inhibitor. In some embodiments, the subject's hematocrit levels are at least 20% less than hematocrit levels in a healthy subject prior to administration of the GlyT1 inhibitor. In some embodiments, the subject's hematocrit levels are at least 30% less than hematocrit levels in a healthy subject prior to administration of the GlyT1 inhibitor. In some embodiments, the subject's hematocrit levels are at least 40% less than hematocrit levels in a healthy subject prior to administration of the GlyT1 inhibitor. In some embodiments, the subject's hematocrit levels are at least 50% less than hematocrit levels in a healthy subject prior to administration of the GlyT1 inhibitor. In some embodiments, the subject's hematocrit levels are at least 60% less than hematocrit levels in a healthy subject prior to administration of the GlyT1 inhibitor. In some embodiments, the subject's hematocrit levels are at least 70% less than hematocrit levels in a healthy subject prior to administration of the GlyT1 inhibitor. In some embodiments, the subject's hematocrit levels are at least 80% less than hematocrit levels in a healthy subject prior to administration of the GlyT1 inhibitor. In some embodiments, the subject's hematocrit levels are at least 90% less than hematocrit levels in a healthy subject prior to administration of the GlyT1 inhibitor.

In some embodiments, the subject has a red blood cell count that is at least 10%, 20%, 30%, 40%, 50%, 60%, 70%, 80%, or 90% less than a red blood cell count in a healthy subject prior to administration of the GlyT1 inhibitor. In some embodiments, the subject has a red blood cell count that is at least 10% less than a red blood cell count in a healthy subject prior to administration of the GlyT1 inhibitor. In some embodiments, the subject has a red blood cell count that is at least 20% less than a red blood cell count in a healthy subject prior to administration of the GlyT1 inhibitor. In some embodiments, the subject has a red blood cell count that is at least 30% less than a red blood cell count in a healthy subject prior to administration of the GlyT1 inhibitor. In some embodiments, the subject has a red blood cell count that is at least 40% less than a red blood cell count in a healthy subject prior to administration of the GlyT1 inhibitor. In some embodiments, the subject has a red blood cell count that is at least 50% less than a red blood cell count in a healthy subject prior to administration of the GlyT1 inhibitor. In some embodiments, the subject has a red blood cell count that is at least 60% less than a red blood cell count in a healthy subject prior to administration of the GlyT1 inhibitor. In some embodiments, the subject has a red blood cell count that is at least 70% less than a red blood cell count in a healthy subject prior to administration of the GlyT1 inhibitor. In some embodiments, the subject has a red blood cell count that is at least 80% less than a red blood cell count in a healthy subject prior to administration of the GlyT1 inhibitor. In some embodiments, the subject has a red blood cell count that is at least 90% less than a red blood cell count in a healthy subject prior to administration of the GlyT1 inhibitor. In some embodiments, the subject has a red blood cell count less than 4.5×10¹²/L. In some embodiments, the subject has a red blood cell count less than 4.1×10¹²/L.

In some embodiments, the GlyT1 inhibitors disclosed herein increase red blood cell synthesis (also known as erythropoiesis), and may be used to treat a condition associated with decreased red blood cells. In some embodiments, the GlyT1 inhibitors disclosed herein may modulate red blood cell synthesis by reducing the formation of heme. In some embodiments, the disclosure relates to methods of increasing red blood cell synthesis in a subject with anemia associated with a ribosomal disorder, comprising administering to a subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In some embodiments, the red blood cell synthesis is increased in a dose dependent manner. In some embodiments, the red blood cell count is increased in a dose dependent manner. In some embodiments, merely by way of non-limiting example, GlyT1 inhibitors may be administered directly to a subject to increase red blood count, if desired. Red blood count may also be reflected by a person's hematocrit (i.e., packed cell volume (PCV) or erythrocyte volume fraction (EVF)), which is the proportion or percentage of blood volume that is occupied by red blood cells. A normal hematocrit is normally about 49% for men and about 48% for women. A lower hematocrit value indicates a lower number of red blood cells.

In certain embodiments, administration of a GlyT1 inhibitor (e.g., bitopertin) to such a subject increases their red blood cell count or hematocrit. Also included are methods of increasing red blood cells in a subject, and methods of increasing hematocrit in a subject, including a subject that has a lower than normal red blood cell count or hematocrit, or is at risk for developing such a condition, comprising administering to the subject a GlyT1 inhibitor (e.g., bitopertin) of the present disclosure, and thereby increasing red blood cell count or hematocrit in the subject.

In some embodiments, the method increases the subject's red blood cell count. In some embodiments, the method increases the subject's red blood cell count by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method increases the subject's red blood cell count by at least 15%. In some embodiments, the method increases the subject's red blood cell count by at least 20%. In some embodiments, the method increases the subject's red blood cell count by at least 25%. In some embodiments, the method increases the subject's red blood cell count by at least 30%. In some embodiments, the method increases the subject's red blood cell count by at least 35%. In some embodiments, the method increases the subject's red blood cell count by at least 40%. In some embodiments, the method increases the subject's red blood cell count by at least 45%. In some embodiments, the method increases the subject's red blood cell count by at least 50%. In some embodiments, the method increases the subject's red blood cell count by at least 55%. In some embodiments, the method increases the subject's red blood cell count by at least 60%. In some embodiments, the method increases the subject's red blood cell count by at least 65%. In some embodiments, the method increases the subject's red blood cell count by at least 70%. In some embodiments, the method increases the subject's red blood cell count by at least 75%. In some embodiments, the method increases the subject's red blood cell count by at least 80%. In some embodiments, the method increases the subject's red blood cell count by at least 85%. In some embodiments, the method increases the subject's red blood cell count by at least 90%. In some embodiments, the method increases the subject's red blood cell count by at least 95%. In some embodiments, the method increases the subject's red blood cell count by at least 100%. In some embodiments, the method increases the subject's red blood cell count to normal levels. In some embodiments, the method increases the subject's red blood cell count to between 4.5-5.9×10¹²/L. In some embodiments, the method increases the subject's red blood cell count to between 4.1-5.1×10¹²/L.

In some embodiments, the method increases the subject's hematocrit levels. In some embodiments, the method increases the subject's hematocrit levels by at least 10% (e.g., 10%, 15%, 20%, 25%, 30%, 35%, 40%, 45%, 50%, 55%, 60%, 65%, 705%, 80%, 85%, 90%, 95%, or at least 100%). In some embodiments, the method increases the subject's hematocrit levels by at least 15%. In some embodiments, the method increases the subject's hematocrit levels by at least 20%. In some embodiments, the method increases the subject's hematocrit levels by at least 25%. In some embodiments, the method increases the subject's hematocrit levels by at least 30%. In some embodiments, the method increases the subject's hematocrit levels by at least 35%. In some embodiments, the method increases the subject's hematocrit levels by at least 40%. In some embodiments, the method increases the subject's hematocrit levels by at least 45%. In some embodiments, the method increases the subject's hematocrit levels by at least 50%. In some embodiments, the method increases the subject's hematocrit levels by at least 55%. In some embodiments, the method increases the subject's hematocrit levels by at least 60%. In some embodiments, the method increases the subject's hematocrit levels by at least 65%, In some embodiments, the method increases the subject's hematocrit levels by at least 70%. In some embodiments, the method increases the subject's hematocrit levels by at least 75%. In some embodiments, the method increases the subject's hematocrit levels by at least 80%. In some embodiments, the method increases the subject's hematocrit levels by at least 85%. In some embodiments, the method increases the subject's hematocrit levels by at least 90%. In some embodiments, the method increases the subject's hematocrit levels by at least 95%. In some embodiments, the method increases the subject's hematocrit levels by at least 100%. In some embodiments, the method increases the subject's hematocrit levels to at least 38%. In some embodiments, the method increases the subject's hematocrit levels to at least 35%.

### Reticulocyte Count and Hemoglobin

In certain embodiments, the present disclosure relates to methods of administering a GlyT1 inhibitor disclosed herein to a subject in need thereof, wherein the subject has a decreased reticulocyte (e.g., less than 1%, often by a clinically or statistically significant amount), or decreased hemoglobin levels (e.g., less than about 13.2 g/dL for men or about 11.6 g/dL for women, often by a clinically or statistically significant amount).

In some embodiments, the subject has hemoglobin levels that are at least 10%, 20%, 30%, 40%, 50%, 60%, 70%, 80%, or 90% less than hemoglobin levels in a healthy subject prior to administration of the GlyT1 inhibitor. In some embodiments, the subject has hemoglobin levels that are at least 10% less than hemoglobin levels in a healthy subject prior to administration of the GlyT1 inhibitor. In some embodiments, the subject has hemoglobin levels that are at least 20% less than hemoglobin levels in a healthy subject prior to administration of the GlyT1 inhibitor. In some embodiments, the subject has hemoglobin levels that are at least 30% less than hemoglobin levels in a healthy subject prior to administration of the GlyT1 inhibitor. In some embodiments, the subject has hemoglobin levels that are at least 40% less than hemoglobin levels in a healthy subject prior to administration of the GlyT1 inhibitor. In some embodiments, the subject has hemoglobin levels that are at least 50% less than hemoglobin levels in a healthy subject prior to administration of the GlyT1 inhibitor. In some embodiments, the subject has hemoglobin levels that are at least 60% less than hemoglobin levels in a healthy subject prior to administration of the GlyT1 inhibitor. In some embodiments, the subject has hemoglobin levels that are at least 70% less than hemoglobin levels in a healthy subject prior to administration of the GlyT1 inhibitor. In some embodiments, the subject has hemoglobin levels that are at least 80% less than hemoglobin levels in a healthy subject prior to administration of the GlyT1 inhibitor. In some embodiments, the subject has hemoglobin levels that are at least 90% less than hemoglobin levels in a healthy subject prior to administration of the GlyT1 inhibitor. In some embodiments, the subject has hemoglobin levels that are less than 13 g/dL. In some embodiments, the subject has hemoglobin levels that are less than 11 g/dL. In some embodiments, the subject has elevated fetal hemoglobin levels.

In some embodiments, the subject has a low reticulocyte count, also known as reticulocytopenia. In some embodiments, the subject has a reticulocyte count of less than 1%. In some embodiments, the subject has a reticulocyte count of less than 0.9%. In some embodiments, the subject has a reticulocyte count of less than 0.8%. In some embodiments, the subject has a reticulocyte count of less than 0.7%. In some embodiments, the subject has a reticulocyte count of less than 0.6%. In some embodiments, the subject has a reticulocyte count of less than 0.5%. In some embodiments, the subject has a reticulocyte count of less than 0.4%. In some embodiments, the subject has a reticulocyte count of less than 0.3%. In some embodiments, the subject has a reticulocyte count of less than 0.2%. In some embodiments, the subject has a reticulocyte count of less than 0.1%.

In certain embodiments, administration of a GlyT1 inhibitor (e.g., bitopertin) to such a subject increases their reticulocyte or hemoglobin levels. Also included are methods of increasing reticulocytes in a subject, and methods of increasing hemoglobin levels in a subject, including a subject that has a lower than normal reticulocyte or hemoglobin levels, or is at risk for developing such a condition, comprising administering to the subject a GlyT1 inhibitor (e.g., bitopertin) of the present disclosure, and thereby reducing reticulocyte or hemoglobin levels in the subject.

In some embodiments, the GlyT1 inhibitors disclosed herein increase hemoglobin synthesis in a subject with anemia associated with a ribosomal disorder, and may be used to treat a condition associated with decreased red blood cells. In some embodiments, the GlyT1 inhibitors disclosed herein may modulate hemoglobin synthesis by reducing the formation of heme. In some embodiments, the disclosure relates to methods of increasing hemoglobin synthesis in a subject with anemia associated with a ribosomal disorder, comprising administering to a subject a pharmaceutical composition comprising one or more glycine transporter inhibitor (e.g., a GlyT1 inhibitor), or a pharmaceutically acceptable salt thereof, or a prodrug of the one or more glycine transporter inhibitor (e.g., a GlyT1 inhibitor) or its salt. In some embodiments, the hemoglobin synthesis is increased in a dose dependent manner.

In some embodiments, the method increases the subject's hemoglobin levels. In some embodiments, the method increases the subject's hemoglobin levels by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method increases the subject's hemoglobin levels by at least 15%. In some embodiments, the method increases the subject's hemoglobin levels by at least 20%. In some embodiments, the method increases the subject's hemoglobin levels by at least 25%. In some embodiments, the method increases the subject's hemoglobin levels by at least 30%. In some embodiments, the method increases the subject's hemoglobin levels by at least 35%. In some embodiments, the method increases the subject's hemoglobin levels by at least 40%. In some embodiments, the method increases the subject's hemoglobin levels by at least 45%. In some embodiments, the method increases the subject's hemoglobin levels by at least 50%. In some embodiments, the method increases the subject's hemoglobin levels by at least 55%. In some embodiments, the method increases the subject's hemoglobin levels by at least 60%. In some embodiments, the method increases the subject's hemoglobin levels by at least 65%. In some embodiments, the method increases the subject's hemoglobin levels by at least 70%. In some embodiments, the method increases the subject's hemoglobin levels by at least 75%. In some embodiments, the method increases the subject's hemoglobin levels by at least 80%. In some embodiments, the method increases the subject's hemoglobin levels by at least 85%. In some embodiments, the method increases the subject's hemoglobin levels by at least 90%. In some embodiments, the method increases the subject's hemoglobin levels by at least 95%. In some embodiments, the method increases the subject's hemoglobin levels by at least 100%. In some embodiments, the method increases the subject's hemoglobin levels to at least 13 g/dL. In some embodiments, the method increases the subject's hemoglobin levels to at least 11 g/dL.

In some embodiments, the method increases the subject's reticulocyte count. In some embodiments, the method increases the subject's reticulocyte count to between 1% to 2%. In some embodiments, the method increases the subject's reticulocyte count to at least 0.5%. In some embodiments, the method increases the subject's reticulocyte count to at least 0.6%. In some embodiments, the method increases the subject's reticulocyte count to at least 0.7%. In some embodiments, the method increases the subject's reticulocyte count to at least 0.8%. In some embodiments, the method increases the subject's reticulocyte count to at least 0.9%. In some embodiments, the method increases the subject's reticulocyte count to at least 1%. In some embodiments, the method increases the subject's reticulocyte count to at least 1.5%. In some embodiments, the method increases the subject's reticulocyte count to at least 2%. In some embodiments, the method increases the subject's reticulocyte count by 0.5%. In some embodiments, the method increases the subject's reticulocyte count by 1%.

### Combination Therapies

Certain embodiments may include combination therapies for treating anemia associated with a ribosomal disorder, including the administration of one or more GlyT1 inhibitors disclosed herein, in combination with other therapeutic agents or treatment modalities. Examples of combination therapies include, without limitation, any one or more additional active agents and/or supportive therapies selected from the group consisting of: trifluoperazine, HDAC inhibitors, glucocorticoids, sotatercept, luspatercept, iron chelators, blood transfusion, platelet transfusion, allogeneic hematopoietic stem cell transplant, autologous gene therapy, lenalidomide (REVLIMID®), and antibiotics. In some embodiments, the method further comprises administering another therapeutic agent to treat the ribosomal protein defect, selected from the group consisting of: corticosteroids and bone marrow transplants and other treatments known to persons of ordinary skill in the art. For instance, corticosteroids can be used to treat anemia associated with a ribosomal disorder, such as DBA. Blood transfusions can also be used to treat severe anemia associated with a ribosomal disorder, such as DBA. Periods of remission may occur, during which transfusions and steroid treatments are not required. Bone marrow transplantation (BMT) can treat hematological aspects of DBA. However, adverse events in transfusion patients can occur. In some embodiments, the method reduces the need for corticosteroid treatments in the subject. In some embodiments, the method reduces the dose of corticosteroid treatment needed in the subject. In some embodiments, the corticosteroid is a glucocorticoid steroid.

As described above, a common therapy for treating anemia associated with a ribosomal disorder includes the use of regularly scheduled blood transfusions. In some embodiments, the GlyT1 inhibitors disclosed herein are useful in treating a subject who has anemia associated with a ribosomal disorder (e.g., Diamond-Blackfan anemia) requiring blood transfusions. In some embodiments, the method reduces the subject's need for blood transfusions. In some embodiments, the method reduces the subject's need for blood transfusions by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method reduces the subject's need for blood transfusions by at least 15%. In some embodiments, the method reduces the subject's need for blood transfusions by at least 20%, In some embodiments, the method reduces the subject's need for blood transfusions by at least 25%. In some embodiments, the method reduces the subject's need for blood transfusions by at least 30%. In some embodiments, the method reduces the subject's need for blood transfusions by at least 35%. In some embodiments, the method reduces the subject's need for blood transfusions by at least 40%. In some embodiments, the method reduces the subject's need for blood transfusions by at least 45%. In some embodiments, the method reduces the subject's need for blood transfusions by at least 50%. In some embodiments, the method reduces the subject's need for blood transfusions by at least 55%. In some embodiments, the method reduces the subject's need for blood transfusions by at least 60%. In some embodiments, the method reduces the subject's need for blood transfusions by at least 65%. In some embodiments, the method reduces the subject's need for blood transfusions by at least 70%. In some embodiments, the method reduces the subject's need for blood transfusions by at least 75%. In some embodiments, the method reduces the subject's need for blood transfusions by at least 80%. In some embodiments, the method reduces the subject's need for blood transfusions by at least 85%. In some embodiments, the method reduces the subject's need for blood transfusions by at least 90%. In some embodiments, the method reduces the subject's need for blood transfusions by at least 95%. In some embodiments, the method reduces the subject's need for blood transfusions by at least 100%. In some embodiments, the method eliminates the subject's need for blood transfusions.

### Quality of Life and Survival

In certain aspects, the disclosure relates to methods of preventing, treating, or reducing the progression rate and/or severity of anemia associated with a ribosomal disorder (e.g., treating, preventing, or reducing the progression rate and/or severity of one or more complications of anemia associated with a ribosomal disorder) comprising administering to a patient in need thereof an effective amount of a GlyT1 inhibitor (e.g., bitopertin), wherein the method increases the patient's quality of life by at least 1% (e.g., 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%). In some embodiments, the method relates to increasing the patient's quality of life. In some embodiments, the method relates to increasing the patient's quality of life by at least 1%. In some embodiments, the method relates to increasing the patient's quality of life by at least 2%. In some embodiments, the method relates to increasing the patient's quality of life by at least 3%. In some embodiments, the method relates to increasing the patient's quality of life by at least 4%. In some embodiments, the method relates to increasing the patient's quality of life by at least 5%. In some embodiments, the method relates to increasing the patient's quality of life by at least 10%. In some embodiments, the method relates to increasing the patient's quality of life by at least 15%. In some embodiments, the method relates to increasing the patient's quality of life by at least 20%. In some embodiments, the method relates to increasing the patient's quality of life by at least 25%. In some embodiments, the method relates to increasing the patient's quality of life by at least 30%. In some embodiments, the method relates to increasing the patient's quality of life by at least 35%. In some embodiments, the method relates to increasing the patient's quality of life by at least 40%. In some embodiments, the method relates to increasing the patient's quality of life by at least 45%. In some embodiments, the method relates to increasing the patient's quality of life by at least 50%. In some embodiments, the method relates to increasing the patient's quality of life by at least 55%. In some embodiments, the method relates to increasing the patient's quality of life by at least 60%. In some embodiments, the method relates to increasing the patient's quality of life by at least 65%. In some embodiments, the method relates to increasing the patient's quality of life by at least 70%. In some embodiments, the method relates to increasing the patient's quality of life by at least 75%. In some embodiments, the method relates to increasing the patient's quality of life by at least 80%. In some embodiments, the method relates to increasing the patient's quality of life by at least 85%. In some embodiments, the method relates to increasing the patient's quality of life by at least 90%. In some embodiments, the method relates to increasing the patient's quality of life by at least 95%. In some embodiments, the method relates to increasing the patient's quality of life by at least 100%. In some embodiments, the patients has a low quality of life.

In some embodiments, the patient's quality of life is measured using the Functional Assessment of Cancer Therapy Anemia (FACT-An). In some embodiments, the patient's quality of life is measured using the Functional Assessment of Cancer Therapy Fatigue (FACT-Fatigue). In some embodiments, the patient's quality of life is measured using the Functional Assessment of Chronic Illness Therapy (FACIT). In some embodiments, the patient's quality of life is measured using the Functional Assessment of Chronic Illness Therapy Fatigue (FACIT-Fatigue). In some embodiments, the patient's quality of life is measured using the Functional Assessment of Chronic Illness Therapy Anemia (FACIT-Anemia). In some embodiments, the patient's quality of life is measured using the SF-36 generic PRO tool. In some embodiments, the patient's quality of life is measured using the SF-6D generic PRO tool. In some embodiments, the patient's quality of life is measured using the linear analog scale assessment (LASA).

In certain aspects, the disclosure relates to methods of preventing, treating, or reducing the progression rate and/or severity of anemia associated with a ribosomal disorder (e.g., treating, preventing, or reducing the progression rate and/or severity of one or more complications of anemia associated with a ribosomal disorder) comprising administering to a patient in need thereof an effective amount of a GlyT1 inhibitor (e.g., bitopertin), wherein the method increases the patient's survival by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method increases the patient's survival. In some embodiments, the method increases the patient's survival by at least 15%. In some embodiments, the method increases the patient's survival by at least 20%. In some embodiments, the method increases the patient's survival by at least 25%. In some embodiments, the method increases the patient's survival by at least 30%. In some embodiments, the method increases the patient's survival by at least 35%. In some embodiments, the method increases the patient's survival by at least 40%. In some embodiments, the method increases the patient's survival by at least 45%. In some embodiments, the method increases the patient's survival by at least 50%. In some embodiments, the method increases the patient's survival by at least 55%. In some embodiments, the method increases the patient's survival by at least 60%. In some embodiments, the method increases the patient's survival by at least 65%. In some embodiments, the method increases the patient's survival by at least 70%. In some embodiments, the method increases the patient's survival by at least 75%. In some embodiments, the method increases the patient's survival by at least 80%. In some embodiments, the method increases the patient's survival by at least 85%. In some embodiments, the method increases the patient's survival by at least 90%. In some embodiments, the method increases the patient's survival by at least 95%. In some embodiments, the method increases the patient's survival by at least 100%.

In some embodiments, the method increases the patient's survival by at least 1 month. In some embodiments, the method increases the patient's survival by at least 2 months. In some embodiments, the method increases the patient's survival by at least 3 months. In some embodiments, the method increases the patient's survival by at least 4 months. In some embodiments, the method increases the patient's survival by at least 5 months. In some embodiments, the method increases the patient's survival by at least 6 months. In some embodiments, the method increases the patient's survival by at least 7 months. In some embodiments, the method increases the patient's survival by at least 8 months. In some embodiments, the method increases the patient's survival by at least 9 months. In some embodiments, the method increases the patient's survival by at least 10 months. In some embodiments, the method increases the patient's survival by at least 11 months.

In some embodiments, the method increases the patient's survival by at least 1 year. In some embodiments, the method increases the patient's survival by at least 2 years. In some embodiments, the method increases the patient's survival by at least 3 years. In some embodiments, the method increases the patient's survival by at least 4 years. In some embodiments, the method increases the patient's survival by at least 5 years. In some embodiments, the method increases the patient's survival by at least 6 years. In some embodiments, the method increases the patient's survival by at least 7 years. In some embodiments, the method increases the patient's survival by at least 8 years. In some embodiments, the method increases the patient's survival by at least 9 years. In some embodiments, the method increases the patient's survival by at least 10 years.

### EXEMPLIFICATION

The invention now being generally described, it will be more readily understood by reference to the following examples, which are included merely for purposes of illustration of certain embodiments of the present invention, and are not intended to limit the invention.

### Example 1: Synthesis of Compounds

The compounds disclosed herein can be made in accordance with well known procedures and by processes known and disclosed in the art. For example, compounds of Formula I, such as bitopertin, can be prepared in accordance with the synthetic protocols provided in U.S. Pat. Nos. 7,319,099, 9,877,963, and 7,812,161, the contents of which are hereby incorporated by reference in their entirety. In addition, compounds of Formula II, such as PF-3463275, can be prepared in accordance with the synthetic protocols provided in U.S. Pat. No. 8,124,639, the contents of which are hereby incorporated by reference in its entirety.

### Example 2: Establishment of TF-1/RPS19 Knock Down Stable Cell Lines

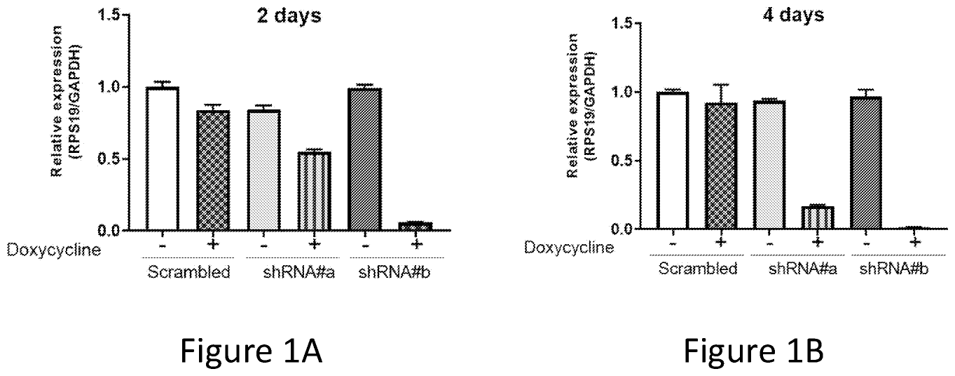

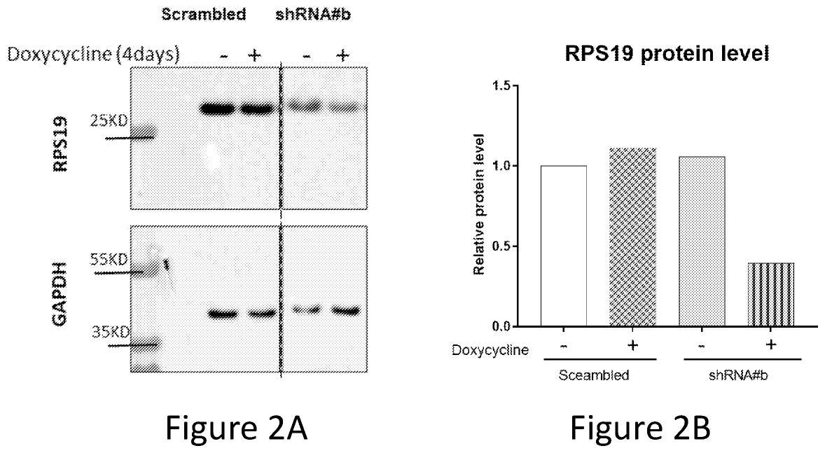

To generate an RPS19 deficient Diamond blackfan anemia (DBA) model, a TF-1 erythroid cell line was transduced with lentivirus encoding shRNAs targeting RPS19 (referred to as “shRNA #a” and “shRNA #b”) and a scrambled shRNA control (referred to as “Scramble shRNA” or “Scrambled”) (See Table 1). Stable cell lines were generated by selecting the infected cells with puromycin (1 ug/ml) for three weeks. The shRNA expression is doxycycline inducible. In the stable cell lines, RPS19 mRNA expression was measured using qRT-PCR after two (FIG. 1A) and four (FIG. 1B) days of doxycycline treatment. The qPCR primers used are described in Table 1. shRNA #a and shRNA #b progressively induced knockdown of RPS19, decreasing RPS19 mRNA expression by >85% by day 4 (FIG. 1A and FIG. 1B). Further, RPS19 protein levels were decreased by more than 70% by shRNA #b by day 5 (FIG. 2A and FIG. 2B). The antibodies used in determining the RPS19 protein levels are described in Table 1.

TABLE 1

| shRNA | sequence |
|---|---|
| RPS19-shRNA#a | GAGATCTGGACAGAATCGC (SEQ ID NO: 1) |
| RPS19-shRNA#b | GCTTGCTCCCTACGATGAGAA (SEQ ID NO: 2) |
| Scramble shRNA | GACACGCGACTTGTACCAC (SEQ ID NO: 3) |
| qPCR Primer | sequence |
| Human RPS19-qPCR1-F | AGACGTGAACCAGCAGGAGT (SEQ ID NO: 4) |
| Human RPS19-qPCR1-R | TTCTCTGACGTCCCCCATAG (SEQ ID NO: 5) |
| Antibody | Source, #Cat, Dilution |
| Anti-RPS19 | Abcam, #ab181365, 1:10000 |
| GAPDH | Cell signaling Technology, #5174, 1:1000 |

### Example 3: Knocking Down RPS19 in TF-1 Erythroid Cells Reduces Cell Growth

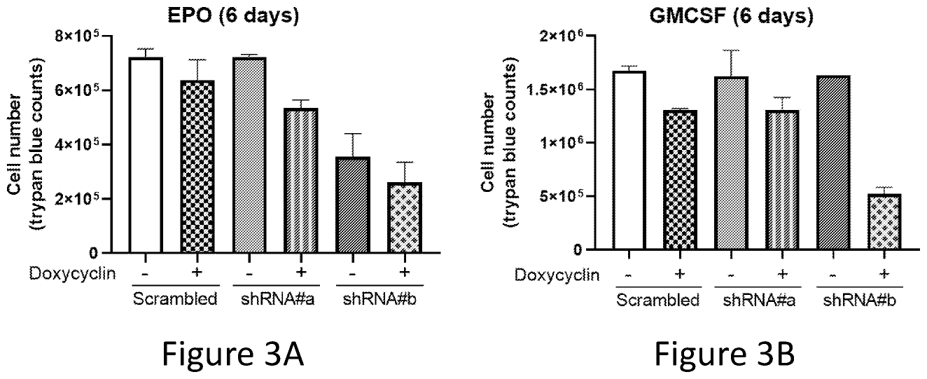

RPS19 knockdown in TF-1 cells has been previously reported to adversely affect the growth of erythroid cells due to an imbalance in heme and globin synthesis. See, e.g., Yang, Z. et al. Sci Transl Med 8, 338ra67 (2016). The TF-1 cell line is a cell line of immature erythroid origin that requires cytokines such as granulocyte-macrophage colony-stimulating factor (GMCSF) or erythropoietin (EPO) for its growth. The cell growth capability of TF-1 stable lines shRNA #a, shRNA #b, and scrambled shRNA were assessed in vitro by cell counting after 6-days of cell culture in the presence of doxycycline induction. Cells were washed from regular growth media and seeded in equal numbers into media containing either GMCSF (2 ng/ml), a growth factor that induces proliferation of the TF-1 cells, or EPO (Ing/ml), a hormone that induces growth of the TF-1 cells and induces their differentiation along the erythroid lineage. Cell growth was monitored by counting cells with trypan blue staining (FIG. 3A and FIG. 3B). The data in FIG. 3A and FIG. 3B demonstrates a RPS19 dependent, moderate effect on cell proliferation in shRNA #a (lower knockdown efficiency) and more substantial effect in shRNA #b (high knockdown efficiency) compared to scrambled shRNA in GMCSF condition (FIG. 3B). EPO is a weak inducer of cell proliferation in TF-1 cells, and in this condition, we also observe dose-dependent growth inhibition of shRNA #a and shRNA #b (FIG. 3A).

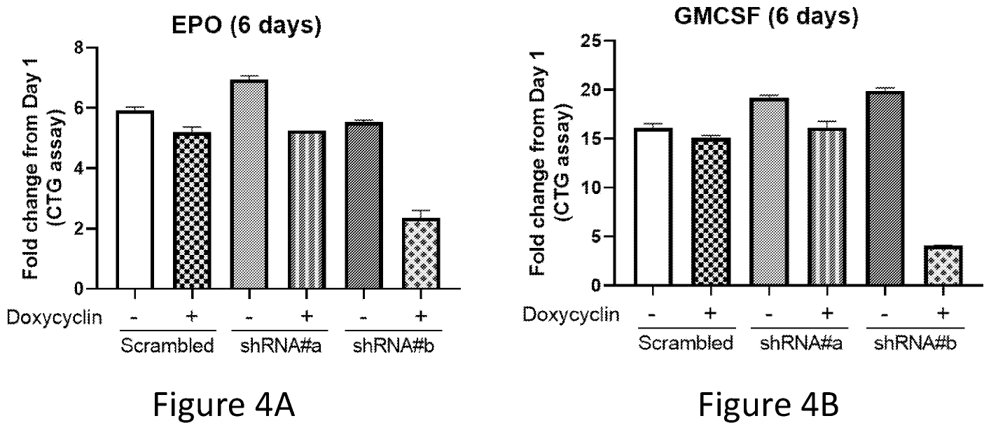

Similarly, the cell growth capability of TF-1 stable lines shRNA #a, shRNA #b, and scrambled shRNA was assessed in vitro by cell viability assay. As described above, washed cells were seeded into 96 well plates in equal numbers in media containing either GMCSF or EPO (FIG. 4A and FIG. 4B) on separate plates for each day of readout. Cell viability was measured using the CellTiter-Glo® (CTG), which determines the number of viable cells in culture by quantifying ATP, which indicates the presence of metabolically active cells. Like the cell count method, moderate cell growth inhibitory effects were observed in shRNA #a, and a high degree inhibition in cell growth was observed in shRNA #b compared to similarly doxycycline-treated scrambled shRNA expressing TF-1 cells in response to strong proliferative GMCSF and weaker EPO stimulus (FIG. 4A and FIG. 4B).

Thus, knocking down RPS19 in the TF-1 erythroid cells resulted in reduced cell growth as determined by the cell counting and the cell viability assay.

### Example 4: Treatment with Bitopertin Increases Cell Growth in RPS19 Knockdown of TF-1 Cells

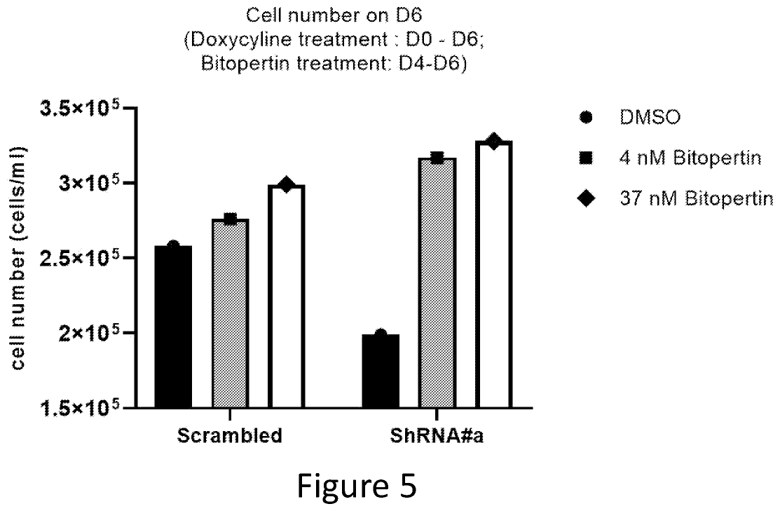

We investigated if blocking the uptake of heme biosynthesis pathway precursor glycine into cells with bitopertin could restore the balance between heme and globin and reverse the anti-proliferative effects caused by RPS19 knockdown. To test this hypothesis, similarly as above, washed cells were seeded in 6-well plates with doxycycline and GMSCSF to induce shRNA expression and cell proliferation for four days. On day 4, 1×10⁵ cells were seeded into a 12-well plate with 4 nM or 37 nM bitopertin. After two days of treatment with bitopertin, we enumerated cell numbers. Bitopertin did not affect TF-1 cells expressing scrambled shRNA; however, a protective effect on TF-1 shRNA #a cells (low knockdown) was achieved even with 4 nM of bitopertin (FIG. 5).

### Example 5: Treatment with Bitopertin Increased Cell Viability in RPS19 Knockdown TF-1 Cells

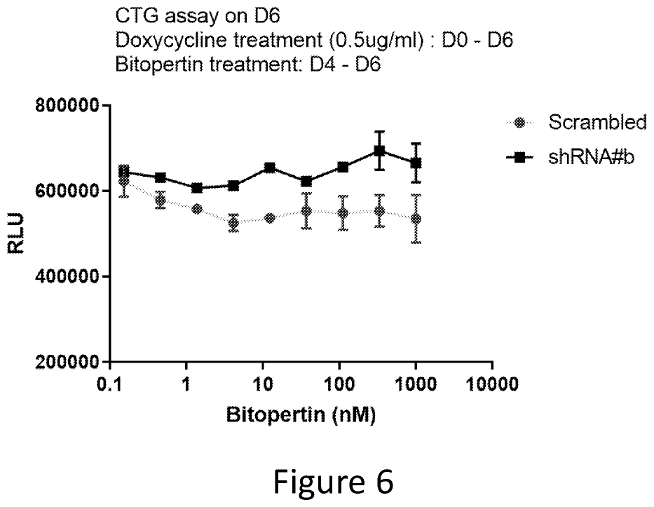

Further, in a similar setup, an equal number of cells were seeded on to 96-well plates after four days of doxycycline treatment and incubated with varying doses of bitopertin (1 μM top concentration, 9 points 3-fold dilutions) for two days and on day 6, we performed a CTG assay to measure the cell viability. The cells were cultured in the presence of GMCSF during the entire cell culture period. A dose-dependent bitopertin protective effect was observed in TF-1/shRNA #b cells compared to TF-1/scrambled shRNA cells (FIG. 6).

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

1. A method of treating Diamond-Blackfan anemia in a subject, the method comprising administering to the subject a pharmaceutical composition comprising a compound having formula: 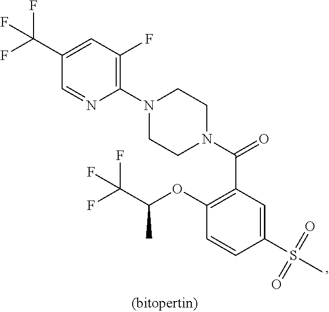 [SMILES: C[C@H](Oc1ccc(S(C)(=O)=O)cc1C(=O)N1CCN(c2ncc(C(F)(F)F)cc2F)CC1)C(F)(F)F]
or a pharmaceutically acceptable salt thereof.

2-38. (canceled)

39. The method of claim 1, wherein the subject has impaired hematopoiesis.

40-48. (canceled)

49. The method of claim 1, wherein the subject has elevated erythrocyte adenosine deaminase activity.

50. The method of claim 1, wherein the subject has macrocytic anemia.

51. The method of claim 1, wherein the subject has reticulocytopenia.

52. (canceled)

53. The method of claim 1, wherein the subject has normal marrow cellularity with a paucity of red cell precursors.

54. The method of claim 1, wherein the subject has normal neutrophil and/or platelet counts.

55. The method of claim 1, wherein the subject has elevated fetal hemoglobin levels.

56. (canceled)

57. The method of claim 1, wherein the method reduces the heme levels in the subject by at least 30%.

58. The method of claim 1, wherein the method reduces heme synthesis in the subject by at least 30%.

59. The method of claim 1, wherein the method reduces intracellular heme levels.

60. The method of claim 1, wherein the method reduces intracellular heme levels in erythroid precursors.

61. (canceled)

62. The method of claim 1, wherein the method increases the subject's red blood cell count.

63-66. (canceled)

67. The method of claim 1, wherein the method increases the subject's hemoglobin levels.

68-73. (canceled)

74. The method of claim 1, wherein the method increases the subject's hematocrit levels.

75-77. (canceled)

78. The method of claim 1, wherein the method reduces anemia in the subject.

79. (canceled)

80. The method of claim 1, wherein the method increases the subject's reticulocyte count.

81. The method of claim 1, wherein the method increases the subject's reticulocyte count to between 1% to 2%.

82. (canceled)

83. The method of claim 1, wherein the method increases the subject's erythroid precursor survival by at least 30%.

84. The method of claim 1, wherein the method increases erythroid precursor differentiation into mature red blood cells in the subject.

85. (canceled)

86. The method of claim 1, wherein the method reduces the risk of heme toxicity in the subject.

87. (canceled)

88. The method of claim 1, wherein the method reduces the risk of liver iron overload.

89. The method of claim 1, wherein the method reduces the levels of iron in the liver.

90. (canceled)

91. The method of claim 1, wherein the method reduces the risk of cardiac iron overload.

92. The method of claim 1, wherein the method reduces the level of iron in the heart.

93-94. (canceled)

95. The method of claim 1, wherein the method reduces the subject's spleen size.

96. (canceled)

97. The method of claim 1, wherein the method reduces the subject's need for blood transfusions.

98-102. (canceled)

103. The method of claim 1, wherein the method reduces the need for corticosteroid treatments in the subject.

104-105. (canceled)

106. The method of claim 1, wherein the method increases survival by at least 30%.

107. The method of claim 1, comprising further administering to the subject an additional active agent and/or supportive therapy.

108. The method of claim 107, wherein the additional active agent and/or supportive therapy is selected from the group consisting of: trifluoperazine, lenalidomide, HDAC inhibitors, glucocorticoids, sotatercept, luspatercept, iron chelators, blood transfusion, platelet transfusion, allogeneic hematopoietic stem cell transplant, autologous gene therapy, and antibiotics.

109-110. (canceled)

111. The method of claim 1, wherein the pharmaceutical composition further comprises a pharmaceutically acceptable carrier.

112-113. (canceled)
